(19) United States
(12) Patent Application Publication
(10) Pub. No.: US 2024/0327911 A1
(43) Pub. Date: Oct. 3, 2024

(54) ANTIGEN REACTIVE T-CELL RECEPTORS

(71) Applicants: Deutsches Krebsforschungszentrum Stiftung des öffentlichen Rechts, Heidelberg (DE); Universität Heidelberg, Heidelberg (DE)

(72) Inventors: Rienk OFFRINGA, Heidelberg (DE); Zibo MENG, Heidelberg (DE); Aaron RODRIGUEZ EHRENFRIED,, Heidelberg (DE); Laura Katharina STEFFENS, Heidelberg (DE); Chin Leng TAN, Heidelberg (DE)

(21) Appl. No.: 18/552,115

(22) PCT Filed: Mar. 23, 2022
(86) PCT No.: PCT/EP2022/057673

§ 371 Date: Sep. 22, 2023

(30) Foreign Application Priority Data
Mar. 23, 2021 (EP) 21164371.3

(51) Int. Cl. C12Q 1/6881; A61K 39/00; C12N 5/0783; C12Q 1/6886; G01N 33/68

(52) U.S. Cl. CPC C12Q 1/6881; A61K 39/4611; C12N 5/0636; C12Q 1/6886; G01N 33/6872; C12Q 2600/158; G01N 2333/7051

(57) ABSTRACT

The present invention relates to a method of identifying a T-cell reactive to cells presenting a T-cell activating antigen (cancer-reactive T-cell), comprising (a) determining expression of at least one of CCL4, CCL4L2, CCL3, CCL3L1, and CXCL13 in T-cells from a sample of a subject; and (b) identifying a cancer-reactive T-cell based on the determination of step (a). The present invention also relates to a method of identifying a TCR binding to a cancer cell of a subject, said method comprising (A) identifying a cancer reactive T-cell according to the afore-said method (B) providing the amino acid sequences of at least the complementarity determining regions (CDRs) of the TCR of the cancer-reactive T-cell identified in step (A); and, hereby, (C) identifying a TCR binding to a cancer cell. The present invention further relates to further methods and cancer-reactive T-cells related thereto.

### CROSS REFERENCE TO RELATED APPLICATIONS

This application is a U.S. National Phase Application, claiming priority to International Patent Application No. PCT/EP2022/057673, filed on Mar. 23, 2022, which claims priority to EP Application No. 21164371.3 filed on Mar. 23, 2021, both of which are hereby incorporated by reference in their entireties.

### INCORPORATION OF SEQUENCE LISTING

A computer readable form of the Sequence Listing containing the file named-“3529373.0004 Sequence Listing_ST25.txt,” which is 8,122 bytes in size (as measured in MICROSOFT WINDOWS® EXPLORER) and was created on Jun. 5, 2024, is provided herein and is herein incorporated by reference. This Sequence Listing consists of SEQ ID NOs: 1-4.

The present invention relates to a method of identifying a T-cell reactive to cells of a subject presenting a T-cell activating antigen (reactive T-cell), comprising (a) determining expression of at least one of CCL4, CCL4L2, CCL3, CCL3L1, and CXCL13 in T-cells from a sample of said subject; and (b) identifying a reactive T-cell based on the determination of step (a). The present invention also relates to a method of identifying a TCR binding to an activating antigen presented on a cell, preferably a cancer cell, of a subject, said method comprising (A) identifying a reactive T-cell according to the method of identifying a reactive T-cell, (B) providing the amino acid sequences of at least the complementarity determining regions (CDRs) of the TCR of the reactive T-cell identified in step (A); and, hereby, (C) identifying a TCR binding to an activating antigen presented on a cell. The present invention further relates to further methods and cancer-reactive T-cells related thereto.

Over recent years, there has been increasing interest in identifying antigen-reactive T-cell receptors (TCRs) for personalized Adoptive Cell Therapies (ACT). In such a therapy, a patient's circulating T cells in the blood are harvested, transgenically modified to express a tumor reactive TCR, and then reinfused into the patient.

As a source of T-cells for identifying e.g. tumor-reactive TCRs, tumor-infiltrating lymphocytes (TILs) have been used. Tumor reactive T cells within a TIL population can in theory be identified by their upregulation of known T cell activation biomarkers such as CD69 and Nur77, though in practice the value of TCRs identified by such an approach has been limited.

Further biomarkers of T-cell activation have been described, cf. Cano-Gamez et al. (2020), Nat Comm 11: art. 1801 (doi.org/10.1038/s41467-020-15543-y), Magen et al. (2019), Cell Rep 29(10):3019 (doi.org/10.1016/j.celrep.2019.10.131), and Oh et al. (2020), Cell 181(7):1612 (doi.org/10.1016/j.cell.2020.05.017). Moreover, e.g. biomarkers predicting non-response to immune checkpoint blockade (WO2018/209324) and biomarkers for immunotherapy resistance (WO2019/070755) have been described. Recently, activation markers from tumor infiltrating T lymphocytes were described (WO 2021/188954 A1, Lowery et al. (2022), Science 10.1126/science.ab15447).

T-cell activation has long been acknowledged to involve presentation of an antigen, e.g. an epitope of a polypeptide, in the context of major histocompatibility complexes (MHCs). MHC class I, interacting with TCR complexes comprising the CD8 protein on CD8+ T-cells, is expressed by all nucleate cells, while MHC class II, interacting with TCR complexes comprising the CD4 protein on CD4+ T-cells, is only expressed by professional antigen presenting cells, mostly B-cells and dendritic cells. However, other surface molecules of cells have been found to be involved in T-cell interaction and activation as well (cf e.g. Iwabuchi & van Kaer (2019), Front Immunol 10:1837 (doi: 10.3389/fimmu.2019.01837).

Nonetheless, there is still a need for improved methods for providing T-cells reactive to specific antigens, e.g. cancer antigens, and corresponding TCRs. This problem is solved by the embodiments characterized in the claims and described herein below.

In accordance, the present invention relates to a method of identifying a T-cell reactive to cells of a subject presenting a T-cell activating antigen (reactive T-cell), comprising
(a) determining expression of at least one of CCL4, CCL4L2, CCL3, CCL3L1, and CXCL13 in T-cells from a sample of said subject; and
(b) identifying a reactive T-cell based on the determination of step (a).

Preferably, the present invention relates to a method of identifying a T-cell reactive to cancer cells of a subject (cancer-reactive T-cell), comprising
(a) determining expression of at least one of CCL4, CCL4L2, CCL3, CCL3L1, and CXCL13 in T-cells from a sample of said subject; and
(b) identifying a cancer-reactive T-cell based on the determination of step (a).

In general, terms used herein are to be given their ordinary and customary meaning to a person of ordinary skill in the art and, unless indicated otherwise, are not to be limited to a special or customized meaning. As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more”, i.e. are equivalent to “comprising at least one”. In accordance, expressions relating to one item of a plurality, unless otherwise indicated, preferably relate to at least one such item, more preferably a plurality thereof: thus, e.g. identifying “a cell” relates to identifying at least one cell, preferably to identifying a multitude of cells.

Further, as used in the following, the terms “preferably”, “more preferably”, “most preferably”, “particularly”, “more particularly”, “specifically”, “more specifically”, or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment”, “in a further embodiment”, or similar expressions are intended to be optional features, without any restriction regarding further embodiments of the invention, without any restrictions regarding the scope of the invention and without any restriction regarding the possibility of combining the features introduced in such way with other optional or non-optional features of the invention.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value ±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1% by weight, most preferably less than 0.1% by weight of non-specified component(s).

The degree of identity (e.g. expressed as “% identity”) between two biological sequences, preferably DNA, RNA, or amino acid sequences, can be determined by algorithms well known in the art. Preferably, the degree of identity is determined by comparing two optimally aligned sequences over a comparison window, where the fragment of sequence in the comparison window may comprise additions or deletions (e.g., gaps or overhangs) as compared to the sequence it is compared to for optimal alignment. The percentage is calculated by determining, preferably over the whole length of the polynucleotide or polypeptide, the number of positions at which the identical residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman (1981), by the homology alignment algorithm of Needleman and Wunsch (1970), by the search for similarity method of Pearson and Lipman (1988), by computerized implementations of these algorithms (GAP, BESTFIT, BLAST, PASTA, and TFASTA in the Wisconsin Genetics Software Package, Genetics Computer Group (GCG), 575 Science Dr., Madison, WI), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. In the context of biological sequences referred to herein, the term “essentially identical” indicates a % identity value of at least 80%, preferably at least 90%, more preferably at least 98%, most preferably at least 99%. As will be understood, the term essentially identical includes 100% identity. The aforesaid applies to the term “essentially complementary” mutatis mutandis.

The term “fragment” of a biological macromolecule, preferably of a polynucleotide or polypeptide, is used herein in a wide sense relating to any sub-part, preferably subdomain, of the respective biological macromolecule comprising the indicated sequence, structure and/or function. Thus, the term includes sub-parts generated by actual fragmentation of a biological macromolecule, but also sub-parts derived from the respective biological macromolecule in an abstract manner, e.g. in silico. In the context of sequence information, in particular nucleic acid sequences and/or polypeptide sequences, the term “sub-sequence” is used for sequences representing only a part of a longer sequence.

Unless specifically indicated otherwise herein, the compounds specified, in particular polynucleotides, polypeptides, or fragments thereof, e.g. variable regions of a T-cell receptor (TCR), may be comprised in larger structures, e.g. may be covalently or non-covalently linked to accessory molecules, carrier molecules, retardants, and other excipients. In particular, polypeptides as specified may be comprised in fusion polypeptides comprising further peptides, which may serve e.g. as a tag for purification and/or detection, as a linker, or to extend the in vivo half-life of a compound. The term “detectable tag” refers to a stretch of amino acids which are added to or introduced into the fusion polypeptide; preferably, the tag is added C- or N-terminally to the fusion polypeptide of the present invention. Said stretch of amino acids preferably allows for detection of the fusion polypeptide by an antibody which specifically recognizes the tag; or it preferably allows for forming a functional conformation, such as a chelator; or it preferably allows for visualization, e.g. in the case of fluorescent tags. Preferred detectable tags are the Myc-tag, FLAG-tag, 6-His-tag, HA-tag, GST-tag or a fluorescent protein tag, e.g. a GFP-tag. These tags are all well known in the art. Other further peptides preferably comprised in a fusion polypeptide comprise further amino acids or other modifications which may serve as mediators of secretion, as mediators of blood-brain-barrier passage, as cell-penetrating peptides, and/or as immune stimulants. Further polypeptides or peptides to which the polypeptides may be fused are signal and/or transport sequences and/or linker sequences. A variable region of a TCR, preferably, is comprised in a backbone of a TCR alpha or beta chain as specified herein below.

The term “polypeptide”, as used herein, refers to a molecule consisting of several, typically at least 20 amino acids that are covalently linked to each other by peptide bonds. Molecules consisting of less than 20 amino acids covalently linked by peptide bonds are usually considered to be “peptides”. Preferably, the polypeptide comprises of from 50 to 1000, more preferably of from 75 to 750, still more preferably of from 100 to 500, most preferably of from 110 to 400 amino acids. Preferably, the polypeptide is comprised in a fusion polypeptide and/or a polypeptide complex.

The method of identifying a reactive T-cell of the present invention, preferably, is an in vitro method. The method may comprise further steps in addition to those related to herein above. For example, further steps may relate, e.g., to providing a sample for step a), or determining further biomarkers in step b). Moreover, one or more of said steps may be performed or assisted by automated equipment.

The term “T-cell receptor”, abbreviated as “TCR”, as used herein, relates to a polypeptide complex on the surface of T-cells mediating recognition of antigenic peptides presented by target cells, preferably in the context of MHC molecules or MHC-related molecules such as MR1 or CD1, more preferably in the context of MHC molecules, still more preferably in the context of MHC class I or MHC class II molecules, most preferably in the context of MHC class I molecules. Typically, the TCR comprises one TCR-alpha chain and one TCR-beta chain, i.e. is an alpha/beta chain heterodimer. The TCR may, however, also comprise a TCR gamma and a TCR delta chain instead of the TCR alpha and beta chains. The TCR alpha and beta or gamma and delta chains mediate antigen recognition and each comprise a transmembrane region, a constant region, a joining region, and a variable region, the variable region of each TCR alpha, beta, gamma, or delta chain comprising three complementarity determining regions (CDRs), referred to as CDR1, CDR2, and CDR3, respectively. In accordance with usual nomenclature, the complex consisting of an alpha and a beta chain or a gamma and a delta chain is referred to as “T-cell receptor” or “TCR” herein, the alpha and/or beta chain and the gamma and/or delta chains commonly or singly being referred to a “TCR polypeptide” or “TCR polypeptides”, whereas the polypeptide complex comprising a TCR and accessory polypeptides, such as CD3 and CD247, is referred to as “T-cell receptor complex”, abbreviated as “TCR complex”. Preferably the T-cell receptor binds to a major histocompatibility complex (MHC) molecule, preferably an MHC class I or class II, more preferably an MHC class I molecule, presenting an antigen contributing and/or associated with disease, preferably a cancer antigen or an autoimmune T-cell antigen, more preferably a cancer antigen, still more preferably an epitope of a cancer specific antigen, in particular a neoepitope of a cancer cell. Binding of a T-cell receptor to an antigen can be determined by methods known to the skilled person, e.g. by methods as specified herein in the Examples, or e.g. in a tetramer assay. Preferably, binding of the TCR to an epitope presented on an MHC activates the T-cell. Activation biomarkers of various types of T-cells are known in the art and include in particular CD69, CD137, CD27, TRAP/CD40L, and CD134. The TCR may also be a soluble TCR. The term “soluble TCR” is, in principle, known to the skilled person to relate to a TCR as specified herein above lacking the transmembrane domains. Thus, preferably, the soluble TCR comprises the constant and the variable regions of the TCR polypeptides of a TCR. More preferably, the soluble TCR comprises the variable regions of the TCR polypeptides of a TCR, preferably in the form of a fusion polypeptide.

The term “complementarity determining region”, abbreviated as “CDR”, is understood by the skilled person. As is known in the art, each TCR alpha, beta, gamma, and delta chain comprises three CDRs, which are the peptides providing the epitope-specificity determining contacts of a TCR to a peptide presented by an MHC molecule as specified elsewhere herein.

The term “T-cell” is understood by the skilled person to relate to a lymphocyte expressing at least one type of T-cell receptor as specified herein above. Preferably, the T-cell is a CD8+ T-cell recognizing MHC class I molecules on the surface of target cells, or is a CD4+ T-cell recognizing MHC class II molecules on the surface of target cells, more preferably is a CD8+ T-cell. Preferably, the T-cell is a cytotoxic T-cell, more preferably a CD8+ cytotoxic T-cell, which may also be referred to as “killer cell”. Also preferably, the T-cell is a regulatory or helper T-cell, more preferably a regulatory T-cell. Preferably, the T-cell is an alpha/beta T-cell, i.e. a T-cell expressing a T-cell receptor comprising a TCR alpha and a TCR beta chain. Preferably, the T-cell is reactive to cells presenting a T-cell activating antigen, i.e. is a “reactive T-cell”, more preferably is specifically reactive to cells presenting a T-cell activating antigen; thus, the T-cell preferably is activated by cells presenting a T-cell activating antigen, preferably is specifically activated by cells presenting a T-cell activating antigen, the terms “specifically activated by cells presenting a T-cell activating antigen” and “specifically reactive to cells presenting a T-cell activating antigen” indicating that the T-cell preferably is activated by cells presenting a T-cell activating antigen, but not by cells not presenting a T-cell activating antigen, in particular of the same tissue. Activation of T-cells can be measured by methods known in the art, e.g. by measuring cytokine secretion, e.g. interferon-gamma secretion, or by a method as specified herein in the Examples. Preferably, the T-cell is reactive to cancer cells, i.e. is a “cancer-reactive T-cell” or is reactive to cells presenting a T-cell autoantigen, i.e. is an “autoimmune-reactive T-cell”. Thus, preferably, the T-cell expresses a TCR recognizing a cancer antigen, preferably a cancer-specific antigen, as specified herein below. In accordance with the above, a T-cell reactive to cancer cells is a T-cell expressing a TCR recognizing a cancer antigen, preferably a cancer-specific antigen. Also preferably, the T-cell expresses a TCR recognizing an autoimmune T-cell antigen, preferably a specific autoimmune T-cell antigen.

The term “T-cell activating antigen”, for which also the expression “activating antigen” may be used, is used herein in a broad sense to relate to any structure presented on the surface of a cell of a subject which can activate a T-cell expressing an appropriate TCR. Preferably, the antigen is a polypeptide or fragment thereof, a polysaccharide, or a lipid. More preferably, the antigen is an epitope of a polypeptide presented by said cell of said subject in the context of an MHC molecule, preferably as specified herein above. As the skilled person understands, if a reactive T-cell is identified by the method as specified herein in a sample, there preferably is a presumption that there are cells in said subject presenting a T-cell activating antigen; since this identification not necessarily includes identifying the T-cell activating antigen, the reactive T-cell identified and/or its TCR may be used further for identifying the T-cell activating antigen. Preferably, the T-cell activating antigen is a cancer antigen or an autoimmune-related T-cell activating antigen. Thus, the reactive T-cell may in particular be a cancer-reactive T-cell or an autoimmune-reactive T-cell.

The term “cancer”, as used herein, relates to a disease of an animal, including man, characterized by uncontrolled growth by a group of body cells (“cancer cells”). This uncontrolled growth may be accompanied by intrusion into and destruction of surrounding tissue and possibly spread of cancer cells to other locations in the body. Preferably, also included by the term cancer is a relapse. Thus, preferably, the cancer is a solid cancer, a metastasis, or a relapse thereof. Cancer may be induced by an infectious agent, preferably a virus, more preferably an oncogenic virus, more preferably Epstein-Barr virus, a hepatitis virus, Human T-lymphotropic virus 1, a papillomavirus, or Human herpesvirus 8. Cancer may, however, also be induced by chemical compounds, e.g. a carcinogen, or endogenously, e.g. caused by spontaneous mutation.

Preferably, the cancer is selected from the list consisting of acute lymphoblastic leukemia, acute myeloid leukemia, adrenocortical carcinoma, aids-related lymphoma, anal cancer, appendix cancer, astrocytoma, atypical teratoid, basal cell carcinoma, bile duct cancer, bladder cancer, brain stem glioma, breast cancer, burkitt lymphoma, carcinoid tumor, cerebellar astrocytoma, cervical cancer, chordoma, chronic lymphocytic leukemia, chronic myelogenous leukemia, colon cancer, colorectal cancer, craniopharyngioma, endometrial cancer, ependymoblastoma, ependymoma, esophageal cancer, extracranial germ cell tumor, extragonadal germ cell tumor, extrahepatic bile duct cancer, fibrosarcoma, gallbladder cancer, gastric cancer, gastrointestinal stromal tumor, gestational trophoblastic tumor, hairy cell leukemia, head and neck cancer, hepatocellular cancer, hodgkin lymphoma, hypopharyngeal cancer, hypothalamic and visual pathway glioma, intraocular melanoma, kaposi sarcoma, laryngeal cancer, medulloblastoma, medulloepithelioma, melanoma, merkel cell carcinoma, mesothelioma, mouth cancer, multiple endocrine neoplasia syndrome, multiple myeloma, mycosis fungoides, nasal cavity and paranasal sinus cancer, nasopharyngeal cancer, neuroblastoma, non-hodgkin lymphoma, non-small cell lung cancer, oral cancer, oropharyngeal cancer, osteosarcoma, ovarian cancer, ovarian epithelial cancer, ovarian germ cell tumor, ovarian low malignant potential tumor, pancreatic cancer, papillomatosis, paranasal sinus and nasal cavity cancer, parathyroid cancer, penile cancer, pharyngeal cancer, pheochromocytoma, pituitary tumor, pleuropulmonary blastoma, primary central nervous system lymphoma, prostate cancer, rectal cancer, renal cell cancer, retinoblastoma, rhabdomyosarcoma, salivary gland cancer, sezary syndrome, small cell lung cancer, small intestine cancer, soft tissue sarcoma, squamous cell carcinoma, squamous neck cancer, testicular cancer, throat cancer, thymic carcinoma, thymoma, thyroid cancer, urethral cancer, uterine sarcoma, vaginal cancer, vulvar cancer, waldenström macroglobulinemia, and wilms tumor. More preferably, the cancer is a solid cancer, a metastasis, or a relapse thereof. More preferably, said cancer is glioblastoma, pancreatic ductal adenocarcinoma, osteosarcoma, or a brain metastasis of a non-brain primary tumor. In a preferred embodiment, the cancer is pancreatic cancer, colorectal cancer, or any other primary or metastatic solid tumor type, preferably is pancreatic cancer or colorectal cancer.

The term “cancer antigen” relates to an antigen, preferably a polypeptide, expressed by a cancer cell. Preferably, the cancer antigen is expressed at an at least 5 fold, preferably at least 10 fold, more preferably at least 25 fold, lower rate in non-cancer cells. Preferably, the cancer antigen is not expressed in non-tumor cells of the same tissue in a subject, more preferably is not expressed in non-cancer cells of a subject; thus, the cancer antigen preferably is a cancer specific antigen. More preferably, the cancer antigen is a neoantigen and/or comprises a neoepitope, expressed by cancer cells. Preferably, one or more peptides of the cancer antigen are presented via MHC molecules, more preferably MHC class-I molecules, on the surface of host cells producing said cancer antigen as “cancer epitopes”, which preferably are cancer-specific epitopes or, as specified above, cancer neoepitopes. As specified elsewhere herein, the cancer preferably is a solid cancer, i.e. a tumor-forming cancer; thus, the cancer antigen preferably is a tumor antigen, more preferably a tumor-specific antigen, and the cancer epitope preferably is a tumor epitope, more preferably a tumor-specific epitope.

The term “autoimmune T-cell activating antigen” is, in principle, known to the skilled person to relate to any antigen presented by a cell of a subject, the recognition of which causes, aggravates, or contributes to autoimmune disease, preferably T-cell mediated autoimmune disease. T-cell mediated autoimmune diseases are known in the art; preferably, the T-cell mediated autoimmune disease is selected from the list consisting of multiple sclerosis, celiac disease, rheumatoid arthritis, type 1 diabetes mellitus, hypothyroidism, and Addison's disease. As the skilled person understands, identification of autoimmune-reactive T-cells and/or their TCRs as proposed herein preferably is particularly suitable for diagnosing, contributing to diagnosing, and/or predicting T-cell mediated autoimmune disease. The autoimmune-reactive T-cells and/or their TCRs may, however, also be used for generation of regulatory T-cells and, therefore, be used in the treatment of T-cell mediated autoimmune disease. Furthermore, the autoimmune-reactive T-cells and/or their TCRs preferably are used in the identification of new autoimmune T-cell activating antigens.

As used herein, the term “host cell” relates to any cell capable of expressing, and preferably presenting on its surface, a TCR polypeptide as specified herein, preferably encoded by a polynucleotide and/or vector. Preferably, the cell is a bacterial cell, more preferably a cell of a common laboratory bacterial strain known in the art, most preferably an Escherichia strain, in particular an E. coli strain. Also preferably, the host cell is a eukaryotic cell, preferably a yeast cell, e.g. a cell of a strain of baker's yeast, or is an animal cell. More preferably, the host cell is an insect cell or a mammalian cell, in particular a mouse or rat cell. Most preferably, the host cell is a human cell. Preferably, the host cell is a T-cell, more preferably a CD8+ T-cell or a CD4+ T-cell, more preferably a CD8+ T-cell. As the skilled person understands, a CD4 TCR is preferably expressed in a CD8+T-call, and a CD4 TCR is preferably expressed in a CD8 T-cell.

The terms “identifying a T-cell reactive to cells presenting a T-cell activating antigen” and “identifying a reactive T-cell”, as used herein, are used in a broad sense including any and all means and methods of providing information on a reactive T-cell allowing determination of at least the CDR sequences of its TCR. In accordance, the reactive T-cell does not have to, but may, be provided in physical form. Thus, identifying a reactive T-cell may comprise identifying a dataset indicative of a T-cell expressing at least one biomarker as specified elsewhere herein and, optionally, allocating at least the CDR sequences of the TCR of said reactive T-cell. Preferably, said dataset is or was determined by single-cell determination of gene expression, preferably by single-cell RNA sequencing. Thus, step a) of the method of identifying a reactive T-cell may comprise performing single-cell determination of gene expression of T-cells in a sample, wherein expression of at least one of the biomarkers as specified is determined, thereby identifying a reactive T-cell; optionally, at least the CDR sequences of the TCR of said T-cell found to express said at least one biomarker are sequenced. Identifying a reactive T-cell may, however, also comprise physically providing said reactive T-cell. Thus, step a) of the method of identifying a reactive T-cell may comprise determining expression of at least one of the biomarkers as specified on and/or in the T-cell. Thus, expression of surface biomarkers may e.g. be determined by antibody staining, optionally followed by FACS-measurements and/or -sorting. Also preferably, single T-cells are grown clonally and biomarker expression is determined in an aliquot of said clonally grown cells. Other methods of determining biomarker expression in a T-cell, preferably a living T-cells, are known in the art.

Determination of expression of a biomarker may be performed based on the amount of any biomarker gene product deemed appropriate by the skilled person. Thus, determination may comprise determining the amount of RNA, in particular mRNA, and/or polypeptide gene product. Expression may, however, also be determined by measuring expression of a surrogate biomarker, e.g. a reporter gene construct in which the reporter gene is expressed under the control of the promoter of the respective biomarker. Preferably, the determination of expression comprises determining the amount of mRNA and/or polypeptide gene product.

Identifying a reactive T-cell comprises determining expression of at least one biomarker as specified elsewhere herein. Expression of a biomarker may be determined qualitatively, semi-quantitatively, or quantitatively, which terms are in principle known to the skilled person. Qualitative determination may be a binary assessment that the biomarker is expressed or not expressed by a T-cell, e.g. by determining whether the biomarker is expressed above a detection level of an assay. Semiquantitative determination may comprise assorting expression to expression categories, such as low, medium, or high expression. The term quantitative determination is understood by the skilled person to include each and every determination providing information on the amount of a biomarker in a cell and all values derived from such an amount by at least one standard mathematical operation, including in particular calculation of a concentration, of a mean, a median, or an average, normalization, and similar calculations.

Preferably, identifying a reactive T-cell comprises comparing biomarker expression determined in a T-cell to a reference. The term “reference”, as used herein, refers to expression of a biomarker in a reference cell, e.g. an amount of biomarker in a reference cell. Preferably, a reference is a threshold value (e.g., an amount or ratio of amounts) for a gene product. The reference may, however, also be a value derived from an amount by any mathematical deemed appropriate by the skilled person, in particular normalization. In accordance with the aforementioned method, a reference is, preferably, a reference obtained from a sample of T-cells known to be reactive T-cells. In such a case, a value for the biomarker gene product found in a sample being essentially identical to said reference is indicative for a reactive T-cell. Also preferably, the reference is from a sample of T-cells known not be reactive. In such a case, a value for the biomarker gene product found in the T-cell to be increased with respect to the reference is indicative for the T-cell being reactive. The same applies mutatis mutandis for a calculated reference, most preferably the average or median, for the relative or absolute value of the biomarker gene product(s) of a population of non-stimulated T-cells. As the skilled person understands, only a small percentage of T-cells of any given natural population of T-cells will be reactive at a time. In accordance, the above description for a population of T-cells known not to be activated may be applied mutatis mutandis to a natural population of T-cells of which the activation status is unknown; thus the reference may be a natural sample of T-cells of which reactivity status is unknown. In such a case, a value for the biomarker gene product found in the T-cell to be increased with respect to the reference is indicative for the T-cell being reactive. How to calculate a suitable reference value, preferably, the average or median, is well known in the art. The population of non-stimulated T-cells referred to before shall comprise a plurality of T-cells, preferably at least 10, more preferably at least 100, still more preferably at least 1,000, most preferably at least 10,000, non-stimulated T-cells. The value for a biomarker gene product of T-cell of interest and the reference values are essentially identical if the corresponding values are essentially identical. Essentially identical means that the difference between two values is, preferably, not significant and shall be characterized in that the values are within at least the interval between 1st and 99th percentile, 5th and 95th percentile, 10th and 90th percentile, 20th and 80th percentile, 30th and 70th percentile, 40th and 60th percentile of the reference value, preferably, the 50th, 60th, 70th, 80th, 90th or 95th percentile of the reference value. Statistical tests for determining whether two amounts are essentially identical are well known in the art. An observed difference for two values, on the other hand, shall preferably be statistically significant. A difference in the relative or absolute value is, preferably, significant outside of the interval between 45th and 55th percentile, 40th and 60th percentile, 30th and 70th percentile, 20th and 80th percentile, 10th and 90th percentile, 5th and 95th percentile, 1st and 99th percentile of the reference value. Preferably, the reference(s) are stored in a suitable data storage medium such as a database and are, thus, also available for future assessments.

Identifying a reactive T-cell comprises determining expression of at least one of CCL4, CCL4L2, CCL3, CCL3L1, and CXCL13, preferably of at least one of CCL4, CCL4L2, CCL3, and CCL3L1. Thus, the method of identifying a reactive T-cell preferably comprises determining expression of at least one biomarker selected from the list consisting of CCL4, CCL4L2, CCL3, CCL3L1, and CXCL13. Thus, the method of identifying a reactive T-cell preferably comprises determining expression of at least one biomarker selected from Table 1 herein below. The aforesaid biomarkers are biomarkers of the “core signature”, i.e. each biomarker alone or any combination thereof is indicative of a reactive T-cell. The aforesaid biomarkers are, in principle, known to the skilled person and their amino acid sequences and sequences of encoding polynucleotides are available from public databases. “CCL4” is also known as “Chemokine (C-C motif) ligand 4” and the amino acid sequence of human CCL4 is available e.g. from Genbank Acc No. NP_996890.1. “CCL4L2” is also known as “C-C motif chemokine 4-like” and the amino acid sequence of human CCL4L2 is available e.g. from Genbank Acc No. NP_001278397.1. “CCL3” is also known as “Chemokine (C-C motif) ligand 3” and may also be referred to as macrophage inflammatory protein 1-alpha (MIP-1-alpha); the amino acid sequence of human CCL3 is available e.g. from Genbank Acc No. NP_002974.1. “CCL3L1” is also known as Chemokine (C-C motif) ligand 3-like 1”, and the amino acid sequence of human CCL3L1 is available e.g. from Genbank Acc No. NP_066286.1. “CXCL13” is also known under the designations “B lymphocyte chemoattractant” and “B cell-attracting chemokine 1”, and the amino acid sequence of human CXCL13 is available e.g. from Genbank Acc No. NP_006410.1. Preferably, expression of at least one of CCL4, CCL4L2, CCL3, CCL3L1, and CXCL13 is indicative of a reactive T-cell. More preferably, expression of at least two, more preferably at least three, most preferably all four of the aforesaid biomarkers is indicative of a reactive T-cell.

In a preferred embodiment, identifying a reactive T-cell comprises determining expression of at least one biomarker from “Gene Set 1” as listed in Table 1, preferably of CXCL13, CCL3, or CXCL13 and CCL3. Preferably, expression of said biomarker(s) is indicative of a reactive T-cell.

Preferably, the method of identifying a reactive T-cell referred to herein comprises further determining expression of at least one biomarker selected from the list consisting of IFNG, HAVCR2, FNBP1, CSRNP1, SPRY1, RHOH, FOXN2, HIF1A, TOB1, RILPL2, CD8B, GABARAPL1, TNFSF14, EGR1, EGR2, TAGAP, TNFSF9, ANXA1, MAP3K8, PIK3R1, DUSP2, DUSP4, DUSP6, CLIC3, RASGEF1B, LAG3, XCL2, NR4A2, DNAJB6, NFKBID, MCL1, EVI2A, SLC7A5, H3F3B, NR4A3, REL, IRF4, CST7, ATF3, TNF, GPR171, BCL2A1, ITGA1, TNFAIP3, NR4A1, RUNX3, HERPUD2, FASLG, CBLB, PTGER4, SLA, XCL1, BHLHE40, LYST, KLRD1, ZNF682, CTSW, SLC2A3, NLRP3, SCML4, VSIR, LINC01871, and ZFP36L1. Thus, the method of identifying a reactive T-cell preferably comprises determining expression of at least one biomarker selected from Table 2 herein below. The biomarkers are biomarkers of the “accessory 1 signature”, i.e. each biomarker of Table 2, alone or in combination with at least one further biomarker of Table 2, is indicative of a reactive T-cell if determined in combination with at least one biomarker of Table 1. Thus, preferably, expression of at least one biomarker of Table 2 in addition to at least one of CCL4, CCL4L2, CCL3, CCL3L1, and CXCL13 is indicative of a reactive T-cell.

Preferably, the method of identifying a reactive T-cell referred to herein comprises further determining expression of at least one biomarker selected from the list consisting of CCL5, GZMH, CLEC2B, GZMA, CD69, GZMK, and CRTAM. Thus, the method of identifying a reactive T-cell preferably comprises determining expression of at least one biomarker selected from Table 3 herein below The biomarkers are biomarkers of the “accessory 2 signature”, i.e. each biomarker of Table 3, alone or in combination with at least one further biomarker of Table 2 or Table 3, is indicative of a reactive T-cell if determined in combination with at least one biomarker of Table 1. Thus, preferably, expression of at least one biomarker of Table 3 in addition to at least one of CCL4, CCL4L2, CCL3, CCL3L1, and CXCL13 is indicative of a reactive T-cell.

In view of the above, all biomarkers of Tables 1 to 3, when expressed in a T-cell, are indicative of a reactive and/or can contribute to identification of a reactive T-cell. Preferably, the method further comprises determination of at least one exclusion biomarker, i.e. a biomarker which, when expressed, is indicative that the T-cell is non-reactive: Preferably, the method of identifying a reactive T-cell referred to herein comprises further determining expression of at least one biomarker selected from the list consisting of GNLY and FGFBP2 (Table 4), wherein expression of at least one of said biomarkers is indicative of a non-reactive T-cell. Thus, the biomarker(s) GNLY and/or FGFBP2 may be used as an exclusion biomarker.

In a preferred embodiment, the method of identifying a reactive T-cell referred to herein comprises further determining expression of at least one, preferably at least two, more preferably at least three, even more preferably at least four, even more preferably at least five, even more preferably at least six, even more preferably at least seven, even more preferably at least eight, even more preferably at least nine, even more preferably at least ten, most preferably all eleven biomarker(s) selected from the list consisting of TNFRSF9, VCAM1, TIGIT, HAVCR2, GZMB, GPR183, CCR7, IL7R, VIM, LTB, and JUNB. Thus, the method of identifying a reactive T-cell preferably comprises determining expression of at least one biomarker selected from Table 5 herein below. The biomarkers of Table 5 in combination with at least one biomarker of Table 1, are biomarkers of “Gene Set 2”. As indicated in Table 5, expression of the markers of Table 5 may be indicative of a reactive T-cell (Prediction “R”), or of a non-reactive T-cell (Prediction “NR”). Preferably, each biomarker of Table 5 labeled “R” alone or in combination with at least one further biomarker of Table 5, is indicative of a reactive T-cell, preferably if determined in combination with at least one biomarker of “Gene Set 1”, i.e. at least one biomarker of Table 1. Also preferably, each biomarker of Table 5 labeled “NR” alone or in combination with at least one further biomarker of Table 5, is indicative of a non-reactive T-cell. Thus, in a preferred embodiment, the method of identifying a reactive T-cell referred to herein comprises determining at least one of TNFRSF9, VCAM1, TIGIT, HAVCR2, GZMB as a marker for tumor-reactive T-cells, and/or at least one of GPR183, CCR7, IL7R, VIM, LTB, JUNB as a marker for non-tumor-reactive T-cells.

In a preferred embodiment, the method of identifying a reactive T-cell referred to herein comprises further determining expression of at least one, preferably at least two, more preferably at least three, even more preferably at least four, even more preferably at least five, even more preferably at least ten, even more preferably at least 15, even more preferably at least 20, even more preferably at least 30, even more preferably at least 40, most preferably all biomarker(s) selected from the list consisting of ACP5, NKG7, KRT86, LAYN, HLA-DRB5, CTLA4, HLA-DRB1, IGFLR1, HLA-DRA, LAG3, GEM, LYST, GAPDH, CD74, HMOX1, HLA-DPA1, DUSP4, CD27, ENTPD1, AC243829.4, HLA-DPB1, GZMH, KIR2DL4, CARD16, HLA-DQA1, CCL5, CST7, LINC01943, PLPP1, CTSC, PRF1, MTSS1, FKBP1A, CXCR6, HLA-DMA, ATP8B4, GZMA, GALNT2, CHST12, SNAP47, TNFRSF18, SIRPG, CD38, RBPJ, TNIP3, AHI1, NDFIP2, FABP5, RAB27A, ADGRG1, CTSW, APOBEC3G, IFNG, CTSD, PKM, NAB1, PSMB9, PARK7, KLRD1, ASXL2, KLRC2, LAIR2, FAM3C, ZFP36, FTH1, FOS, ZFP36L2, ANXA1, CD55, SLC2A3, LMNA, CRYBG1, DUSP1, PTGER4, MYADM, BTG2, and NFKBIA. Thus, the method of identifying a reactive T-cell preferably comprises determining expression of at least one biomarker selected from Table 6 herein below. The biomarkers of Table 6 in combination with at least one biomarker of Table 1, are biomarkers of “Gene Set 3”. As indicated in Table 6, expression of the markers of Table 6 may be indicative of a reactive T-cell (Prediction “R”), or of a non-reactive T-cell (Prediction “NR”). Preferably, each biomarker of Table 6 labeled “R” alone or in combination with at least one further biomarker of Table 5, is indicative of a reactive T-cell, preferably if determined in combination with at least one biomarker of “Gene Set 1”, i.e. at least one biomarker of Table 1, and/or at least one biomarker of Table 5 labeled Prediction “R”. Also preferably, each biomarker of Table 6 labeled “NR” alone or in combination with at least one further biomarker of Table 6, is indicative of a non-reactive T-cell, preferably if determined in combination with at least one biomarker of Table 5 labeled Prediction “NR”. Thus, in a preferred embodiment, the method of identifying a reactive T-cell referred to herein comprises determining at least one of ACP5, NKG7, KRT86, LAYN, HLA-DRB5, CTLA4, HLA-DRB1, IGFLR1, HLA-DRA, LAG3, GEM, LYST, GAPDH, CD74, HMOX1, HLA-DPA1, DUSP4, CD27, ENTPD1, AC243829.4, HLA-DPB1, GZMH, KIR2DL4, CARD16, HLA-DQA1, CCL5, CST7, LINC01943, PLPP1, CTSC, PRF1, MTSS1, FKBP1A, CXCR6, HLA-DMA, ATP8B4, GZMA, GALNT2, CHST12, SNAP47, TNFRSF18, SIRPG, CD38, RBPJ, TNIP3, AHI1, NDFIP2, FABP5, RAB27A, ADGRG1, CTSW, APOBEC3G, IFNG, CTSD, PKM, NAB1, PSMB9, PARK7, KLRD1, ASXL2, KLRC2, LAIR2, and FAM3C as a markers for tumor-reactive T-cells, and/or at least one of ZFP36, FTH1, FOS, ZFP36L2, ANXA1, CD55, SLC2A3, LMNA, CRYBG1, DUSP1, PTGER4, MYADM, BTG2, and NFKBIA as a marker for non-tumor-reactive T-cells.

In view of the above, in a preferred embodiment, the method of identifying a reactive T-cell referred to herein comprises determining expression of at least CXCL13+CCL3+TNFRSF9+VCAM1+TIGIT+HAVCR2+GZMB+GPR183+CCR7+IL7R+VIM; CXCL13+CCL3+TNFRSF9+VCAM1+TIGIT+HAVCR2+GZMB+GPR183+CCR7+IL7R+LTB; CXCL13+CCL3+TNFRSF9+VCAM1+TIGIT+HAVCR2+GZMB+GPR183+CCR7+IL7R+JUNB; CXCL13+CCL3+TNFRSF9+VCAM1+TIGIT+HAVCR2+GZMB+GPR183+CCR7+VIM+LTB; CXCL13+CCL3+TNFRSF9+VCAM1+TIGIT+HAVCR2+GZMB+GPR183+CCR7+VIM+JUNB; CXCL13+CCL3+TNFRSF9+VCAM1+TIGIT+HAVCR2+GZMB+GPR183+CCR7+LTB+JUNB; CXCL13+CCL3+TNFRSF9+VCAM1+TIGIT+HAVCR2+GZMB+GPR183+IL7R+VIM+LTB; CXCL13+CCL3+TNFRSF9+VCAM1+TIGIT+HAVCR2+GZMB+GPR183+IL7R+VIM+JUNB; CXCL13+CCL3+TNFRSF9+VCAM1+TIGIT+HAVCR2+GZMB+GPR183+IL7R+LTB+JUNB; CXCL13+CCL3+TNFRSF9+VCAM1+TIGIT+HAVCR2+GZMB+GPR183+VIM+LTB+JUNB; CXCL13+CCL3+TNFRSF9+VCAM1+TIGIT+HAVCR2+GZMB+CCR7+IL7R+VIM+LTB; CXCL13+CCL3+TNFRSF9+VCAM1+TIGIT+HAVCR2+GZMB+CCR7+IL7R+VIM+JUNB; CXCL13+CCL3+TNFRSF9+VCAM1+TIGIT+HAVCR2+GZMB+CCR7+IL7R+LTB+JUNB; CXCL13+CCL3+TNFRSF9+VCAM1+TIGIT+HAVCR2+GZMB+CCR7+VIM+LTB+JUNB; CXCL13+CCL3+TNFRSF9+VCAM1+TIGIT+HAVCR2+GZMB+IL7R+VIM+LTB+JUNB; CXCL13+CCL3+TNFRSF9+VCAM1+TIGIT+HAVCR2+GPR183+CCR7+IL7R+VIM+LTB; CXCL13+CCL3+TNFRSF9+VCAM1+TIGIT+HAVCR2+GPR183+CCR7+IL7R+VIM+JUNB; CXCL13+CCL3+TNFRSF9+VCAM1+TIGIT+HAVCR2+GPR183+CCR7+IL7R+LTB+JUNB; CXCL13+CCL3+TNFRSF9+VCAM1+TIGIT+HAVCR2+GPR183+CCR7+VIM+LTB+JUNB; CXCL13+CCL3+TNFRSF9+VCAM1+TIGIT+HAVCR2+GPR183+IL7R+VIM+LTB+JUNB; CXCL13+CCL3+TNFRSF9+VCAM1+TIGIT+HAVCR2+CCR7+IL7R+VIM+LTB+JUNB; CXCL13+CCL3+TNFRSF9+VCAM1+TIGIT+GZMB+GPR183+CCR7+IL7R+VIM+LTB; CXCL13+CCL3+TNFRSF9+VCAM1+TIGIT+GZMB+GPR183+CCR7+IL7R+VIM+JUNB; CXCL13+CCL3+TNFRSF9+VCAM1+TIGIT+GZMB+GPR183+CCR7+IL7R+LTB+JUNB; CXCL13+CCL3+TNFRSF9+VCAM1+TIGIT+GZMB+GPR183+CCR7+VIM+LTB+JUNB; CXCL13+CCL3+TNFRSF9+VCAM1+TIGIT+GZMB+GPR183+IL7R+VIM+LTB+JUNB; CXCL13+CCL3+TNFRSF9+VCAM1+TIGIT+GZMB+CCR7+IL7R+VIM+LTB+JUNB; CXCL13+CCL3+TNFRSF9+VCAM1+TIGIT+GPR183+CCR7+IL7R+VIM+LTB+JUNB; CXCL13+CCL3+TNFRSF9+VCAM1+HAVCR2+GZMB+GPR183+CCR7+IL7R+VIM+LTB; CXCL13+CCL3+TNFRSF9+VCAM1+HAVCR2+GZMB+GPR183+CCR7+IL7R+VIM+JUNB; CXCL13+CCL3+TNFRSF9+VCAM1+HAVCR2+GZMB+GPR183+CCR7+IL7R+LTB+JUNB; CXCL13+CCL3+TNFRSF9+VCAM1+HAVCR2+GZMB+GPR183+CCR7+VIM+LTB+JUNB; CXCL13+CCL3+TNFRSF9+VCAM1+HAVCR2+GZMB+GPR183+IL7R+VIM+LTB+JUNB; CXCL13+CCL3+TNFRSF9+VCAM1+HAVCR2+GZMB+CCR7+IL7R+VIM+LTB+JUNB; CXCL13+CCL3+TNFRSF9+VCAM1+HAVCR2+GPR183+CCR7+IL7R+VIM+LTB+JUNB; CXCL13+CCL3+TNFRSF9+VCAM1+GZMB+GPR183+CCR7+IL7R+VIM+LTB+JUNB; CXCL13+CCL3+TNFRSF9+TIGIT+HAVCR2+GZMB+GPR183+CCR7+IL7R+VIM+LTB; CXCL13+CCL3+TNFRSF9+TIGIT+HAVCR2+GZMB+GPR183+CCR7+IL7R+VIM+JUNB; CXCL13+CCL3+TNFRSF9+TIGIT+HAVCR2+GZMB+GPR183+CCR7+IL7R+LTB+JUNB; CXCL13+CCL3+TNFRSF9+TIGIT+HAVCR2+GZMB+GPR183+CCR7+VIM+LTB+JUNB; CXCL13+CCL3+TNFRSF9+TIGIT+HAVCR2+GZMB+GPR183+IL7R+VIM+LTB+JUNB; CXCL13+CCL3+TNFRSF9+TIGIT+HAVCR2+GZMB+CCR7+IL7R+VIM+LTB+JUNB; CXCL13+CCL3+TNFRSF9+TIGIT+HAVCR2+GPR183+CCR7+IL7R+VIM+LTB+JUNB; CXCL13+CCL3+TNFRSF9+TIGIT+GZMB+GPR183+CCR7+IL7R+VIM+LTB+JUNB; CXCL13+CCL3+TNFRSF9+HAVCR2+GZMB+GPR183+CCR7+IL7R+VIM+LTB+JUNB; CXCL13+CCL3+VCAM1+TIGIT+HAVCR2+GZMB+GPR183+CCR7+IL7R+VIM+LTB; CXCL13+CCL3+VCAM1+TIGIT+HAVCR2+GZMB+GPR183+CCR7+IL7R+VIM+JUNB; CXCL13+CCL3+VCAM1+TIGIT+HAVCR2+GZMB+GPR183+CCR7+IL7R+LTB+JUNB; CXCL13+CCL3+VCAM1+TIGIT+HAVCR2+GZMB+GPR183+CCR7+VIM+LTB+JUNB; CXCL13+CCL3+VCAM1+TIGIT+HAVCR2+GZMB+GPR183+IL7R+VIM+LTB+JUNB; CXCL13+CCL3+VCAM1+TIGIT+HAVCR2+GZMB+CCR7+IL7R+VIM+LTB+JUNB; CXCL13+CCL3+VCAM1+TIGIT+HAVCR2+GPR183+CCR7+IL7R+VIM+LTB+JUNB; CXCL13+CCL3+VCAM1+TIGIT+GZMB+GPR183+CCR7+IL7R+VIM+LTB+JUNB; CXCL13+CCL3+VCAM1+HAVCR2+GZMB+GPR183+CCR7+IL7R+VIM+LTB+JUNB; CXCL13+CCL3+TIGIT+HAVCR2+GZMB+GPR183+CCR7+IL7R+VIM+LTB+JUNB; CXCL13+TNFRSF9+VCAM1+TIGIT+HAVCR2+GZMB+GPR183+CCR7+IL7R+VIM+LTB; CXCL13+TNFRSF9+VCAM1+TIGIT+HAVCR2+GZMB+GPR183+CCR7+IL7R+VIM+JUNB; CXCL13+TNFRSF9+VCAM1+TIGIT+HAVCR2+GZMB+GPR183+CCR7+IL7R+LTB+JUNB; CXCL13+TNFRSF9+VCAM1+TIGIT+HAVCR2+GZMB+GPR183+CCR7+VIM+LTB+JUNB; CXCL13+TNFRSF9+VCAM1+TIGIT+HAVCR2+GZMB+GPR183+IL7R+VIM+LTB+JUNB; CXCL13+TNFRSF9+VCAM1+TIGIT+HAVCR2+GZMB+CCR7+IL7R+VIM+LTB+JUNB; CXCL13+TNFRSF9+VCAM1+TIGIT+HAVCR2+GPR183+CCR7+IL7R+VIM+LTB+JUNB; CXCL13+TNFRSF9+VCAM1+TIGIT+GZMB+GPR183+CCR7+IL7R+VIM+LTB+JUNB; CXCL13+TNFRSF9+VCAM1+HAVCR2+GZMB+GPR183+CCR7+IL7R+VIM+LTB+JUNB; CXCL13+TNFRSF9+TIGIT+HAVCR2+GZMB+GPR183+CCR7+IL7R+VIM+LTB+JUNB; CXCL13+VCAM1+TIGIT+HAVCR2+GZMB+GPR183+CCR7+IL7R+VIM+LTB+JUNB; CCL3+TNFRSF9+VCAM1+TIGIT+HAVCR2+GZMB+GPR183+CCR7+IL7R+VIM+LTB; CCL3+TNFRSF9+VCAM1+TIGIT+HAVCR2+GZMB+GPR183+CCR7+IL7R+VIM+JUNB; CCL3+TNFRSF9+VCAM1+TIGIT+HAVCR2+GZMB+GPR183+CCR7+IL7R+LTB+JUNB; CCL3+TNFRSF9+VCAM1+TIGIT+HAVCR2+GZMB+GPR183+CCR7+VIM+LTB+JUNB; CCL3+TNFRSF9+VCAM1+TIGIT+HAVCR2+GZMB+GPR183+IL7R+VIM+LTB+JUNB; CCL3+TNFRSF9+VCAM1+TIGIT+HAVCR2+GZMB+CCR7+IL7R+VIM+LTB+JUNB; CCL3+TNFRSF9+VCAM1+TIGIT+HAVCR2+GPR183+CCR7+IL7R+VIM+LTB+JUNB; CCL3+TNFRSF9+VCAM1+TIGIT+GZMB+GPR183+CCR7+IL7R+VIM+LTB+JUNB; CCL3+TNFRSF9+VCAM1+HAVCR2+GZMB+GPR183+CCR7+IL7R+VIM+LTB+JUNB; CCL3+TNFRSF9+TIGIT+HAVCR2+GZMB+GPR183+CCR7+IL7R+VIM+LTB+JUNB; CCL3+VCAM1+TIGIT+HAVCR2+GZMB+GPR183+CCR7+IL7R+VIM+LTB+JUNB; or TNFRSF9+VCAM1+TIGIT+HAVCR2+GZMB+GPR183+CCR7+IL7R+VIM+LTB+JUNB.

Also in view of the above, in a preferred embodiment, the method of identifying a reactive T-cell referred to herein comprises determining expression of at least CXCL13+CCL3+TNFRSF9+VCAM1+TIGIT+HAVCR2+GZMB+GPR183+CCR7+IL7R+VIM+LTB; CXCL13+CCL3+TNFRSF9+VCAM1+TIGIT+HAVCR2+GZMB+GPR183+CCR7+IL7R+VIM+JUNB; CXCL13+CCL3+TNFRSF9+VCAM1+TIGIT+HAVCR2+GZMB+GPR183+CCR7+IL7R+LTB+JUNB; CXCL13+CCL3+TNFRSF9+VCAM1+TIGIT+HAVCR2+GZMB+GPR183+CCR7+VIM+LTB+JUNB; CXCL13+CCL3+TNFRSF9+VCAM1+TIGIT+HAVCR2+GZMB+GPR183+IL7R+VIM+LTB+JUNB; CXCL13+CCL3+TNFRSF9+VCAM1+TIGIT+HAVCR2+GZMB+CCR7+IL7R+VIM+LTB+JUNB; CXCL13+CCL3+TNFRSF9+VCAM1+TIGIT+HAVCR2+GPR183+CCR7+IL7R+VIM+LTB+JUNB; CXCL13+CCL3+TNFRSF9+VCAM1+TIGIT+GZMB+GPR183+CCR7+IL7R+VIM+LTB+JUNB; CXCL13+CCL3+TNFRSF9+VCAM1+HAVCR2+GZMB+GPR183+CCR7+IL7R+VIM+LTB+JUNB; CXCL13+CCL3+TNFRSF9+TIGIT+HAVCR2+GZMB+GPR183+CCR7+IL7R+VIM+LTB+JUNB; CXCL13+CCL3+VCAM1+TIGIT+HAVCR2+GZMB+GPR183+CCR7+IL7R+VIM+LTB+JUNB; CXCL13+TNFRSF9+VCAM1+TIGIT+HAVCR2+GZMB+GPR183+CCR7+IL7R+VIM+LTB+JUNB; or CCL3+TNFRSF9+VCAM1+TIGIT+HAVCR2+GZMB+GPR183+CCR7+IL7R+VIM+LTB+JUNB.

In a further preferred embodiment, the method of identifying a reactive T-cell referred to herein comprises determining expression of at least CXCL13+CCL3+TNFRSF9+VCAM1+TIGIT+HAVCR2+GZMB+GPR183+CCR7+IL7R+VIM+LTB+JUNB.

The skilled person is aware that the biomarkers referred to herein may be expressed in a plurality of isoforms, from different alleles, and/or may be expressed as precursor forms which may be further processed in the cell, e.g. during intracellular trafficking and/or secretion. Also, the skilled person is aware that subjects from non-human species will preferably express homologues of the specific sequences indicated herein above, which may preferably be identified by sequence alignment and/or search algorithms based thereon, such as the BLAST algorithm, and appropriate databases, preferably publicly available databases. Preferably, the amino acid sequence of a biomarker as specified is at least 50%, more preferably 75%, still more preferably 85%, even more preferably at least 95%, even more preferably at least 98%, most preferably at least 99%, identical to a specific biomarker sequence as referred to herein.

The term “subject”, as used herein, relates to an animal, preferably a vertebrate, more preferably a mammal, preferably to a livestock, like a cattle, a horse, a pig, a sheep, or a goat, to a companion animal, such as a cat or a dog, or to a laboratory animal, like a rat, mouse, or guinea pig. Preferably, the mammal is a primate, more preferably a monkey, most preferably a human. Preferably, the subject is suffering from cancer, in particular in case of the method of identifying a T-cell reactive to cancer cells of a subject. It is, however, also envisaged that the subject is an apparently healthy subject, preferably at least 50 years, more preferably at least 60 years, more preferably at least 70 years, even more preferably at least 80 years of age.

The term “sample” refers to a sample of separated cells or to a sample from a tissue or an organ, preferably from a tumor. Thus, the sample preferably comprises or is assumed to comprise cancer recognizing lymphocytes, preferably T-cells. More preferably, the sample comprises or is assumed to comprise tumor-infiltrating lymphocytes (TILs). Also preferably, the sample comprises cancer cells, more preferably tumor cells. Thus, the sample preferably comprises TILs and cancer cells, preferably is a tumor sample. The sample may, however also be a sample of non-cancer tissue, preferably of cancer-adjacent tissue, or a sample of peripheral blood monocytes (PBMCs). As is known to the skilled person, tissue or organ samples may be obtained from any tissue or organ by, e.g., biopsy, surgery, or any other method deemed appropriate by the skilled person. Separated cells may be obtained from the body fluids, such as lymph, blood, plasma, serum, liquor and other, or from the tissues or organs by separating techniques such as centrifugation or cell sorting. Preferably, the sample is a tissue or body fluid sample which comprises cells. Preferably the sample is a sample of a body fluid, preferably a blood sample. The body fluid sample can be obtained from the subject by routine techniques which are well known to the person skilled in the art, e.g., venous or arterial puncture, lavage, or any other method deemed appropriate by the skilled person.

Advantageously, it was found in the work underlying the present invention that using the biomarkers CCL4, CCL4L2, CCL3, CCL3L1, and/or CXCL13, optionally including further biomarkers, allows identification of T-cells which comprise TCRs which are reactive to antigens presented by cells and, therefore, particularly suitable for providing, either by cultivation or by expressing the respective TCR in a T-cell, T-cells recognizing e.g. cancer cells, e.g. for cellular therapy of cancer.

The definitions made above apply mutatis mutandis to the following. Additional definitions and explanations made further below also apply for all embodiments described in this specification mutatis mutandis.

The present invention further relates to a method of identifying a TCR binding to an activating antigen presented on a cell, preferably a cancer cell, of a subject, said method comprising
(A) identifying a reactive T-cell according to the method of identifying reactive T-cells,
(B) providing the amino acid sequences of at least the complementarity determining regions (CDRs) of the TCR of the reactive T-cell identified in step (A); and, hereby,
(C) identifying a TCR binding to an activating antigen presented on a cell.

The method of identifying a TCR, preferably, is an in vitro method. The method may comprise further steps in addition to those related to herein above. For example, further steps may relate, e.g., to determining further nucleic acid or amino acid sequences, or determining CD8 and/or CD4 expression by said reactive T-cell. Moreover, one or more of said steps may be performed or assisted by automated equipment.

The term “providing a sequence”, such as an amino acid sequence and/or a nucleic acid sequence, is used herein in a broad sense including any and all means and methods of providing information on said sequence or making said sequence information accessible. Thus, the sequence may be provided as a sequence information, preferably tangibly embedded on a data carrier. The sequence may, however, also be provided in the form of a molecule comprising said sequence, preferably as a TCR comprising TCR alpha and beta chains comprising said sequences, more preferably as a host cell comprising the same. As the skilled person understands, if the aforesaid host cell is provided, the sequence information can be provided by standard methods known to the skilled person, e.g. nucleic acid sequencing of the TCR expressed by said host cell or of parts thereof.

The term “identifying a TCR” is used herein in a broad sense including any and all means and methods of providing information on a TCR allowing determination of at least its CDR sequences. In accordance, the TCR does not have to, but may, be provided in physical form. Thus, identifying a TCR may comprise providing at least the CDR sequences of the TCR or of a polynucleotide encoding at least said CDRs. Preferably, said sequences are or were determined by single-cell determination of gene expression, preferably by single-cell RNA sequencing, preferably as specified herein above. Identifying a TCR may, however, also comprise physically providing said TCR, e.g. by providing a host cell, preferably a T-cell, expressing said TCR, or by providing at least one polynucleotide encoding at least the CDRs of the TCR polypeptides identified. As will be understood, in case the TCR is provided in the context of a self-replicating entity such as a host cell, it may not be necessary to provide the amino acid of at least the CDRs of the TCR and/or the nucleic acid complex of a polynucleotide encoding the same.

Preferably, the method of identifying a TCR binding to an activating antigen further comprises step B1) expressing a TCR comprising at least the CDRs determined in step B) in a host cell, preferably a T-cell. More preferably, said method comprises further step B1) expressing a TCR comprising at least the CDRs determined in step B) in a host cell, preferably a T-cell, i.e. preferably comprises expressing a TCR comprising at least the CDRs determined in step B) and at least one accessory TCR polypeptide in a host cell.

The term “TCR comprising at least the CDRs” as specified, as used herein, relates to a TCR in which at least the CDRs are those as determined in step B), while the residual sequences of the TCR polypeptides may be sequences of one or more different alpha and beta or gamma and delta chains, e.g. heterologous sequences. More preferably, the variable regions of the TCR molecules are provided in step B) and are expressed as parts of the TCR polypeptides in step B1). It is, however, also envisaged that sequences of further fragments of the TCR polypeptides or the complete TCR polypeptides are provided in step B), and are optionally expressed in step B1). As the skilled person understands, it is also possible to provide longer sequences in step B) than are expressed in step B1); e.g., preferably, the amino acid sequence of the variable regions of the TCR polypeptides may be provided in step B), while only the CDRs thereof are expressed, in the context e.g. of heterologous TCR polypeptides, in step B1); or, the amino acid sequences of the variable regions of the TCR polypeptides may be provided in step B), and the amino acid sequence of the antigen binding region, including the CDRs, may be expressed, in the context e.g. of heterologous TCR polypeptides, in step B1). If not otherwise indicated, the TCR polypeptides preferably are expressed as complete molecules, i.e. each comprising a transmembrane region, a constant region, a joining region, and a variable region.

Preferably, the method of identifying a TCR binding to a cell presenting a T-cell activating antigen comprises further step B2) determining binding of the TCR expressed in step B1) to a cell presenting a T-cell activating antigen, preferably a cancer antigen, complexed in a major histocompatibility complex (MHC), preferably MHC class I, molecule. Methods of determining binding of a TCR, preferably comprised in a TCR, to a T-cell activating antigen complexed in a major histocompatibility complex (MHC) molecule are known in the art and include, preferably, determining binding of a T-cell activating antigen complexed an MHC molecule carrying a detectable label to the TCR which may e.g. be expressed on the surface of a host cell. A well-known example of such a method is a tetramer assay, preferably using a soluble tetrameric MHC molecule complexed with a T-cell activating antigen.

Preferably, the method identifying a TCR binding to a T-cell activating antigen comprises further step B3) determining recognition of cells presenting said T-cell activating antigen by the TCR expressed in step B1). Assays fur determining such recognition are known in the art and include in particular binding assays, activation assays, and lysis assays. In all of these assays, preferably cells presenting T-cell activating antigen are co-incubated with host cells such as T-cells expressing a TCR comprising at least the CDRs as specified. In a binding assay, it is determined whether the cancer cells and the aforesaid host cells bind to each other, preferably to form an immunological synapse including at least an MHC molecule of the cell presenting a T-cell activating antigen and the TCR. In an activation assay, the host cell, preferably the T-cell, expressing a TCR comprising at least the CDRs as specified, is tested after said co-incubation for biomarkers of immunological activation, e.g. interferon-gamma production. In a lysis assay, it is determined whether the host cells, preferably the T-cells, expressing a TCR comprising at least the CDRs as specified, lysed at least a fraction of the cells presenting the T-cell activating antigen during said co-incubation.

Preferably, the method of identifying a TCR binding to an activating antigen comprises further step B4) producing a soluble TCR comprising at least the CDRs determined in step B) and determining binding of said soluble TCR to a cancer cell and/or to a cancer antigen complexed in a major histocompatibility complex (MHC), preferably MHC class I, molecule. Soluble TCRs have been described herein above. Preferably, soluble TCRs carrying a detectable label are used in step B4); thus, binding of a such labeled soluble TCR may e.g. be detected by fluorescence-activated cell sorting equipment.

Preferably, in the method of identifying a TCR, expression of said at least one biomarker is determined by single-cell determination of gene expression, preferably of at least 100 T-cells, more preferably at least 1000 T-cells. In such case, the amino acid sequences of at least the complementarity determining regions (CDRs) of the TCR of the reactive T-cell of step (B) may be provided as part of the single-cell determination of gene expression, i.e. the mRNAs encoding said CDRs may be sequenced as part of said single-cell determination of gene expression. Preferably, corresponding sequences are pre-amplified before single-cell determination of gene expression. The mRNAs encoding said CDRs may, however, also be determined in separate sequencing steps, preferably by using appropriate barcoding methods. Also in the aforesaid case, the method may comprise further step (B*1) clustering the T-cells based on said gene expression data including the amino acid sequences of said at least CDR sequences and further step (B*2) selecting the TCR or TCRs being clustered at increased relative frequency in clusters expressing said at least one biomarker compared to clusters not expressing said at least one biomarker.

The present invention also relates to a method of providing a T-cell recognizing a cell presenting a T-cell activating antigen, preferably a cancer cell, said method comprising
(i) identifying a TCR binding to a cell presenting a T-cell activating antigen according to the method according to the present invention,
(ii) expressing a TCR comprising at least the complementarity determining regions (CDRs) of the TCR of step (I) in a T-cell, and, thereby,
(iii) providing a T-cell recognizing a cell presenting a T-cell activating antigen, preferably a cancer cell.

The method of providing a T-cell, preferably, is an in vitro method, of which one or more steps may be performed or assisted by automated equipment.

The method may comprise further steps in addition to those related to herein above. For example, further steps may relate, e.g., to cloning at least polynucleotides encoding the CDRs of the TCR of step (i) into a TCR alpha, beta, gamma, or delta chain backbone, or cloning polynucleotides encoding the variable regions of the TCR polypeptides of step (i) into TCR alpha and beta or a TCR gamma and delta, chain backbones, preferably on at least one expression vector; or cloning polynucleotides encoding TCR polynucleotides into one or more expression vectors. As will be understood by the skilled person, CDRs and/or a variable region of a TCR alpha chain will preferably be cloned into a TCR alpha chain backbone; and CDRs and/or a variable region of a TCR beta chain will preferably be cloned into a TCR beta chain backbone. The aforesaid applies mutatis mutandis to gamma and delta chains. The method may also comprise the further step of expanding, preferably clonally expanding, the T-cell recognizing a cancer cell to provide a preparation of cells T-cell recognizing cancer cells. It will thus be appreciated that the T-cell recognizing cancer cells may be the T-cell identified in step (i) or a clonal derivative (i.e. a daughter cell) thereof.

Preferably, the method of providing a T-cell further comprises a step of testing reactivity of the T-cell of step (ii) to cells presenting an activating agent, e.g. a cancer cells. The term “testing reactivity of a T-cell”, as used herein, includes each and every method deemed suitable by the skilled person to determine whether the T-cell as specified is reactive. Preferred methods for testing reactivity have been described herein above, e.g. determining binding, activation of T cells, and/or lysis of cells presenting an activating antigen, e.g. cancer cells.

The present invention further relates to a reactive T-cell identified by the method of identifying a T-cell reactive to cells presenting a T-cell activating antigen, preferably cancer cells, as specified herein above and/or obtained or obtainable by the method of providing a T-cell recognizing a cells presenting a T-cell activating antigen as specified herein above, preferably comprising a T-cell receptor comprising an amino acid sequence of SEQ ID NO:1 and/or SEQ ID NO:2, preferably encoded by a polynucleotide comprising SEQ ID NO:3 and/or 4, respectively, for use in medicine, in particular for use in treating and/or preventing cancer or autoimmune disease in a subject.

The terms “treating” and “treatment” refer to an amelioration of the diseases or disorders referred to herein or the symptoms accompanied therewith to a significant extent. Said treating as used herein also includes an entire restoration of health with respect to the diseases or disorders referred to herein. It is to be understood that treating, as the term is used herein, may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 10%, at least 20% at least 50% at least 60%, at least 70%, at least 80%, or at least 90% of the subjects of a given cohort or population. Preferably, treating cancer is reducing tumor burden in a subject. As will be understood by the skilled person, effectiveness of treatment of e.g. cancer is dependent on a variety of factors including, e.g. cancer stage and cancer type.

The term “preventing” refers to retaining health with respect to the diseases or disorders referred to herein for a certain period of time in a subject. It will be understood that the said period of time may be dependent on the amount of the drug compound which has been administered and individual factors of the subject discussed elsewhere in this specification. It is to be understood that prevention may not be effective in all subjects treated with the compound according to the present invention. However, the term requires that, preferably, a statistically significant portion of subjects of a cohort or population are effectively prevented from suffering from a disease or disorder referred to herein or its accompanying symptoms. Preferably, a cohort or population of subjects is envisaged in this context which normally, i.e. without preventive measures according to the present invention, would develop a disease or disorder as referred to herein. Whether a portion is statistically significant can be determined without further ado by the person skilled in the art using various well known statistic evaluation tools discussed elsewhere in this specification.

The present invention also relates to a pharmaceutical composition comprising a reactive T-cell identified by the method as specified herein above and/or obtained or obtainable by the method of providing a T-cell recognizing a cell presenting a activating antigen as specified herein above, preferably comprising a T-cell receptor comprising an amino acid sequence of SEQ ID NO:1 and/or SEQ ID NO:2.

The term “pharmaceutical composition”, as used herein, relates to a composition comprising the compound or compounds, including host cells, in particular T-cells, as specified herein in a pharmaceutically acceptable form and a pharmaceutically acceptable carrier. The compounds and/or excipients can be formulated as pharmaceutically acceptable salts. Acceptable salts comprise acetate, methylester, HCl, sulfate, chloride and the like. The pharmaceutical compositions are, preferably, administered topically or systemically, preferably intravenously or intratumorally. The compounds can be administered in combination with other drugs either in a common pharmaceutical composition or as separated pharmaceutical compositions wherein said separated pharmaceutical compositions may be provided in form of a kit of parts. In particular, coadministration of adjuvants may be envisaged.

The compounds are, preferably, administered in conventional dosage forms prepared by combining the host cells or drugs with standard pharmaceutical carriers according to conventional procedures. These procedures may involve mixing, dispersing, or dissolving the ingredients as appropriate to the desired preparation. It will be appreciated that the form and character of the pharmaceutically acceptable carrier or diluent is dictated by the amount of active ingredient with which it is to be combined, the route of administration and other well-known variables. The carrier(s) must be acceptable in the sense of being compatible with the other ingredients of the formulation and being not deleterious to the recipient thereof. The pharmaceutical carrier employed may be, for example, either a solid, a gel or, preferably a liquid. Exemplary of liquid carriers are phosphate buffered saline solution, water, emulsions, various types of wetting agents, sterile solutions and the like. Suitable carriers comprise those mentioned above and others well known in the art, see, e.g., Remington's Pharmaceutical Sciences, Mack Publishing Company, Easton, Pennsylvania. The diluent(s) is/are preferably selected so as not to affect the biological activity of the T-cells and potential further pharmaceutically active ingredients. Examples of such diluents are distilled water, physiological saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

A therapeutically effective dose refers to an amount of the compounds to be used in a pharmaceutical composition of the present invention which prevents, ameliorates or treats a condition referred to herein. Therapeutic efficacy and toxicity of compounds can be determined by standard pharmaceutical procedures in cell culture or in experimental animals, e.g., by determining the ED50 (the dose therapeutically effective in 50% of the population) and/or the LD50 (the dose lethal to 50% of the population). The dose ratio between therapeutic and toxic effects is the therapeutic index, and it can be expressed as the ratio, LD50/ED50.

The dosage regimen will be determined by the attending physician, preferably taking into account relevant clinical factors and, preferably, in accordance with any one of the methods described elsewhere herein. As is well known in the medical arts, a dosage for any one patient may depend upon many factors, including the patient's size, body surface area, age, the particular compound to be administered, sex, time and route of administration, general health, and other drugs being administered concurrently. Progress can be monitored by periodic assessment. A typical dose can be, for example, in the range of 10⁴ to 10⁹ host cells; however, doses below or above this exemplary range are envisioned, especially considering the aforementioned factors. The pharmaceutical compositions and formulations referred to herein are administered at least once in order to treat or prevent a disease or condition recited in this specification. However, the said pharmaceutical compositions may be administered more than one time, for example, preferably from one to four times, more preferably two or three times.

The present invention also relates to a polynucleotide encoding at least one TCR binding to an activating antigen provided or identifiable according to the method of identifying a TCR binding to an activating antigen as specified herein.

The term “polynucleotide” is known to the skilled person. As used herein, the term includes nucleic acid molecules comprising or consisting of a nucleic acid sequence or nucleic acid sequences as specified herein. The polynucleotide of the present invention shall be provided, preferably, either as an isolated polynucleotide (i.e. isolated from its natural context) or in genetically modified form. The polynucleotide, preferably, is DNA, including cDNA, or is RNA.

The term encompasses single as well as double stranded polynucleotides. Preferably, the polynucleotide is a chimeric molecule, i.e., preferably, comprises at least one nucleic acid sequence, preferably of at least 20 bp, more preferably at least 100 bp, heterologous to the residual nucleic acid sequences. Moreover, preferably, comprised are also chemically modified polynucleotides including naturally occurring modified polynucleotides such as glycosylated or methylated polynucleotides or artificial modified one such as biotinylated polynucleotides.

The present invention also relates to a method of identifying at least one biomarker of reactive T-cells, comprising
(I) providing expression data of a plurality of biomarkers of T-cells in a sample of a subject,
(II) providing a clustering said plurality of T-cells based on the expression of the biomarkers of step (A);
(III) providing amino acid sequences of at least the complementarity determining regions (CDRs) of TCR chains of T-cells of step (B);
(IV) determining recognition of cancer cells by a TCR comprising the complementarity determining regions (CDRs) of step (C);
(V) repeating steps (C) and (D) at least once for further T-cells clustering with T-cells whose TCRs are determined to recognize cells presenting a T-cell activating antigen in step (D), wherein the TCRs of said further T-cells are non-identical to the TCRs of step (D);
(VI) determining at least one cluster of step (B) comprising the highest fraction of T-cells comprising T-cell receptors recognizing cells presenting a T-cell activating antigen; and
(VII) determining at least one biomarker expressed by the highest fraction of T-cells in the cluster determined in step (F), thereby identifying at least one biomarkers of reactive T-cells.

The method of the present invention, preferably, is an in vitro method. Moreover, it may comprise steps in addition to those explicitly mentioned above. Moreover, one or more of said steps may be aided or performed by automated equipment.

The terms “providing expression data” and “providing amino acid sequences” are understood by the skilled person to include each and every means of making the respective data available. Such data may be provided from pre-existing databases, preferably expression databases. Preferably, providing expression data of a plurality of biomarkers of T-cells comprises determining expression of said biomarkers e.g. by hybridization of RNA or cDNA derived therefrom to an expression array according to methods known in the art. As referred to herein, providing expression data is providing expression data for single cells, i.e. providing expression data of the biomarkers for each cell separately, thus allowing identification sets of biomarkers expressed by a T-cell. Thus, expression data are preferably determined by single-cell determination of gene expression, more preferably by single-cell RNA sequencing, as specified elsewhere herein. Preferably, the expression data comprise the sequences of at least the CDRs of the TCRs expressed by said T-cells. Preferably, the expression data comprise expression data of T-cell activation biomarkers and/or of the biomarkers specified herein above.

The term “providing a clustering” relates to providing an allocation of individual T-cells into clusters sharing similar sets of expressed biomarkers. Said clustering preferably is performed in a computer-implemented manner by an algorithm known in the art, e.g. graph-based clustering or k-mean clustering MacQueen (1967), “Some methods for classification and analysis of multivariate observations”, 5th Berkeley Symposium on Mathematical Statistics and Probability. Clustering may be visualized by methods also known in the art, e.g. tSNE (van der Maaten and Hinton (2008), J Machine Learning Res 9:2579) or UMAP (McInnes et al. (2020), arXiv:1802.03426v3), preferably UMAP. However, other clustering methods may be used as well. Preferably, a multitude of clusters, i.e. at least two, preferably at least five, more preferably at least ten, still more preferably at least 25, is provided. Preferably, the clustering, i.e. the result of the clustering step, is subject-specific.

The term “determining at least one cluster” is understood by the skilled person. According to step (II) of the method, a clustering is provided, preferably providing at least two clusters; according to step (IV), members of the at least two clusters are evaluated whether they are reactive T-cells, and according to step (VI), at least one cluster is determined comprising the highest fraction of T-cells comprising T-cell receptors recognizing cells presenting a T-cell activating antigen. As the skilled person understands, this step preferably identifies the cluster(s) comprising reactive T-cells without the need to know initially which biomarkers are indicative of reactive T-cells. Once a cluster is identified, further T-cell members of the same cluster will preferably be assumed to also be cancer reactive. As will also be understood, repeating steps (III) and (IV) at least once, preferably at least twice, more preferably at least three times, allows for further refining the cluster definition. The biomarkers expressed with the highest frequency in the cluster(s) eventually identified will preferably be assumed to be biomarkers of cancer-reactive T-cells.

The invention further discloses and proposes a computer program including computer-executable instructions for performing the method according to the present invention in one or more of the embodiments enclosed herein when the program is executed on a computer or computer network. Specifically, the computer program may be stored on a computer-readable data carrier. Thus, specifically, one, more than one or even all of method steps a) to d) as indicated above may be performed by using a computer or a computer network, preferably by using a computer program.

The invention further discloses and proposes a computer program product having program code means, in order to perform the method according to the present invention in one or more of the embodiments enclosed herein when the program is executed on a computer or computer network. Specifically, the program code means may be stored on a computer-readable data carrier. Further, the invention discloses and proposes a data carrier having a data structure stored thereon, which, after loading into a computer or computer network, such as into a working memory or main memory of the computer or computer network, may execute the method according to one or more of the embodiments disclosed herein.

The invention further proposes and discloses a computer program product with program code means stored on a machine-readable carrier, in order to perform the method according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier. Specifically, the computer program product may be distributed over a data network.

Finally, the invention proposes and discloses a modulated data signal which contains instructions readable by a computer system or computer network, for performing the method according to one or more of the embodiments disclosed herein.

Preferably, referring to the computer-implemented aspects of the invention, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

Specifically, the present invention further discloses:
A computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer program, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network, and
a computer program product having program code means, wherein the program code means can be stored or are stored on a storage medium, for performing the method according to one of the embodiments described in this description, if the program code means are executed on a computer or on a computer network.

In view of the above, the following embodiments are particularly envisaged:

Embodiment 1: A method of identifying a T-cell reactive to cells of a subject presenting a T-cell activating antigen (reactive T-cell), comprising
(a) determining expression of at least one of CCL4, CCL4L2, CCL3, CCL3L1, and CXCL13 in T-cells from a sample of said subject; and
(b) identifying a reactive T-cell based on the determination of step (a), preferably wherein said T-cell activating antigen is a cancer antigen or an autoimmune T-cell antigen, more preferably is a cancer antigen.

Embodiment 2: A method of identifying a T-cell reactive to cancer cells (cancer-reactive T-cell), comprising
(a) determining expression of at least one of CCL4, CCL4L2, CCL3, CCL3L1, and CXCL13 in T-cells from a sample of a subject; and
(b) identifying a cancer-reactive T-cell based on the determination of step (a).

Embodiment 2: The method of embodiment 1 or 2, wherein step (a) comprises determining expression of at least two, preferably at least three, more preferably at least four, of CCL4, CCL4L2, CCL3, CCL3L1, and CXCL13, preferably of CXCL13 and CCL3.

Embodiment 4: The method of any one of embodiments 1 to 3, wherein step (a) comprises further determining expression of at least one biomarker selected from the list consisting of IFNG, HAVCR2, FNBP1, CSRNP1, SPRY1, RHOH, FOXN2, HIF1A, TOB1, RILPL2, CD8B, GABARAPL1, TNFSF14, EGR1, EGR2, TAGAP, TNFSF9, ANXA1, MAP3K8, PIK3R1, DUSP2, DUSP4, DUSP6, CLIC3, RASGEF1B, LAG3, XCL2, NR4A2, DNAJB6, NFKBID, MCL1, EVI2A, SLC7A5, H3F3B, NR4A3, REL, IRF4, CST7, ATF3, TNF, GPR171, BCL2A1, ITGA1, TNFAIP3, NR4A1, RUNX3, HERPUD2, FASLG, CBLB, PTGER4, SLA, XCL1, BHLHE40, LYST, KLRD1, ZNF682, CTSW, SLC2A3, NLRP3, SCML4, VSIR, LINC01871, and ZFP36L1.

Embodiment 5: The method of any one of embodiments 1 to 4, wherein expression is determined in step (a) by single-cell determination of gene expression, preferably by single-cell RNA sequencing and/or wherein said sample is a tumor sample.

Embodiment 6: A method of identifying a TCR binding to an activating antigen presented on a cell, preferably a cancer cell, of a subject, said method comprising
(A) identifying a reactive T-cell according to the method of any one of embodiments 1 to 5,
(B) providing the amino acid sequences of at least the complementarity determining regions (CDRs) of the TCR of the reactive T-cell identified in step (A); and, hereby,
(C) identifying a TCR binding to an activating antigen presented on a cell.

Embodiment 7: The method of any one of embodiment 1 to 6, wherein at least one biomarker of step a) and/or the nucleic acid sequences in step (B) is/are determined by single-cell sequencing, preferably by single-cell RNA sequencing.

Embodiment 8: The method of embodiment 6 or 7, wherein said method comprises further step B1) expressing a TCR comprising at least the CDRs determined in step B) in a host cell, preferably a T-cell.

Embodiment 9: The method of any one of embodiments 6 to 8, wherein said method comprises further step B1) expressing a TCR comprising at least the CDRs determined in step B) in a host cell, preferably a T-cell.

Embodiment 10: The method of embodiment 9, wherein said method comprises further step B2) determining binding of the TCR expressed in step B1) to an activating antigen presented on a cell, preferably a cancer antigen, complexed in a major histocompatibility complex (MHC), preferably MHC class I, molecule, preferably in a tetramer assay.

Embodiment 11: The method of embodiment 9 or 10, wherein said method further comprises step B3) determining recognition of cells presenting a T-cell activating antigen by the TCR expressed in step B1).

Embodiment 12: The method of any one of embodiments 6 to 11, wherein said method further comprises further step B4) producing a soluble TCR comprising at least the CDRs determined in step B) and determining binding of said soluble TCR to a T-cell activating antigen; preferably to a cancer antigen complexed in a major histocompatibility complex (MHC), preferably MHC class I, molecule.

Embodiment 13: A method of providing a T-cell recognizing a cell presenting a T-cell activating antigen, preferably a cancer cell, said method comprising
(i) identifying a TCR binding to a cell presenting a T-cell activating antigen according to the method according to any one of embodiments 6 to 12,
(ii) expressing a TCR comprising at least the complementarity determining regions (CDRs) of the TCR of step (I) in a T-cell, and, thereby,
(iii) providing a T-cell recognizing a cell presenting a T-cell activating antigen, preferably a cancer cell.

Embodiment 14: The method of embodiment 13, wherein said method further comprises a step of testing reactivity of the T-cell of step (ii) to cells presenting a T-cell activating antigen.

Embodiment 15: The method of any one of embodiments 1 to 14, wherein said sample is a tissue sample or a bodily fluid sample.

Embodiment 16: The method of any one of embodiments 1 to 15, wherein said sample is a blood sample.

Embodiment 17: The method of any one of embodiments 1 to 16, wherein said sample is a cancer sample.

Embodiment 18: The method of any one of embodiments 1 to 17, wherein said sample is a sample of non-cancer tissue, preferably of cancer-adjacent tissue.

Embodiment 19: A reactive T-cell identified by the method according to any one of embodiments 1 to 5 and/or obtained or obtainable by the method according to embodiment 13 or 14, preferably comprising a T-cell receptor comprising an amino acid sequence of SEQ ID NO:1 and/or SEQ ID NO:2, for use in medicine.

Embodiment 20: A reactive T-cell identified by the method according to any one of embodiments 1 to 5 and/or obtained or obtainable by the method according to embodiment 13 or 14, preferably comprising a T-cell receptor comprising an amino acid sequence of SEQ ID NO:1 and/or SEQ ID NO:2, for use in treating and/or preventing cancer in a subject.

Embodiment 21: The subject matter of any one of embodiments 1 to 20, wherein said subject is an apparently healthy subject.

Embodiment 22: The subject matter of any one of embodiments 1 to 21, wherein said subject is a subject afflicted with cancer.

Embodiment 23: The subject matter of any one of embodiments 1 to 22, wherein said cells presenting a T-cell activating antigen are cancer cells, preferably tumor cells.

Embodiment 24: A method of identifying at least one biomarker of reactive T-cells, comprising
(I) providing expression data of a plurality of biomarkers of T-cells in a sample of a subject,
(II) providing a clustering said plurality of T-cells based on the expression of the biomarkers of step (I);
(III) providing amino acid sequences of at least the complementarity determining regions (CDRs) of TCRes of T-cells of step (II);
(IV) determining reactivity of T-cells expressing a TCR comprising the CDRs of step (III) to cells presenting a T-cell activating antigen;
(V) repeating steps (III) and (IV) at least once for further T-cells clustering with T-cells whose TCRs are determined to be reactive to cells presenting a T-cell activating antigen in step (IV), wherein the TCRs of said further T-cells are non-identical to the TCRs of step (IV);
(VI) determining at least one cluster of step (II) comprising the highest fraction of T-cells comprising T-cell receptors recognizing cells presenting a T-cell activating antigen; and
(VII) determining at least one biomarker expressed by the highest fraction of T-cells in the cluster determined in step (VI), thereby identifying at least one biomarkers of cancer-reactive T-cells.

Embodiment 25: The subject matter of any of the preceding embodiments, wherein said T-cell(s) is/are CD8+ T-cell(s) or CD4+ T-cells, preferably are CD8+ T-cells.

Embodiment 26: The subject matter of any of the preceding embodiments, wherein said TCR comprises, preferably consists of, a TCR alpha chain and a TCR beta chain or a TCR gamma chain and a TCR delta chain, preferably comprises, more preferably consists of, a TCR alpha chain and a TCR beta chain.

Embodiment 27: A polynucleotide encoding at least one TCR binding to an activating antigen provided or identifiable according to the method according to any one of embodiments 6 to 12.

Embodiment 28: The subject matter of any of the preceding embodiments, wherein said reactive T-cell is a cancer-reactive T-cell.

Embodiment 29: The method of any one of embodiments 1 to 3, wherein step (a) comprises further determining expression of at least one biomarker selected from the list consisting of TNFRSF9, VCAM1, TIGIT, HAVCR2, GZMB, GPR183, CCR7, IL7R, VIM, LTB, and JUNB.

Embodiment 30: The method of any one of embodiments 1 to 3 and 29, wherein step (a) comprises further determining expression of at least one biomarker selected from the list consisting of ACP5, NKG7, KRT86, LAYN, HLA-DRB5, CTLA4, HLA-DRB1, IGFLR1, HLA-DRA, LAG3, GEM, LYST, GAPDH, CD74, HMOX1, HLA-DPA1, DUSP4, CD27, ENTPD1, AC243829.4, HLA-DPB1, GZMH, KIR2DL4, CARD16, HLA-DQA1, CCL5, CST7, LINC01943, PLPP1, CTSC, PRF1, MTSS1, FKBP1A, CXCR6, HLA-DMA, ATP8B4, GZMA, GALNT2, CHST12, SNAP47, TNFRSF18, SIRPG, CD38, RBPJ, TNIP3, AHI1, NDFIP2, FABP5, RAB27A, ADGRG1, CTSW, APOBEC3G, IFNG, CTSD, PKM, NAB1, PSMB9, PARK7, KLRD1, ASXL2, KLRC2, LAIR2, FAM3C, ZFP36, FTH1, FOS, ZFP36L2, ANXA1, CD55, SLC2A3, LMNA, CRYBG1, DUSP1, PTGER4, MYADM, BTG2, and NFKBIA.

Embodiment 31: The method of any one of embodiments 1 to 3 and 29 to 30, wherein step (a) comprises determining expression of
(i) CXCL13, CCL3 and all biomarkers listed in embodiment 29;
(ii) CXCL13, CCL3 and all biomarkers listed in embodiment 29 except IL7R; or
(iii) CXCL13, CCL3 and all biomarkers listed in embodiment 29 except GPR183.

Embodiment 32: The method of any one of embodiments 1 to 3 and 29 to 31, wherein step (a) comprises determining expression of
(iv) CXCL13, CCL3 and all biomarkers listed in embodiment 29 and embodiment 30;
(v) CXCL13, CCL3 and all biomarkers listed in embodiment 29 except IL7R, as well as all biomarkers listed in embodiment 30; or
(vi) CXCL13, CCL3 and all biomarkers listed in embodiment 29 except GPR183, as well as all biomarkers listed in embodiment 30.

Embodiment 33: The method of any one of embodiments 1 to 3 and 29 to 32, wherein said T-cell activating antigen is a cancer antigen, and wherein preferably said sample is a tumor sample.

Embodiment 34: The method of embodiment 34, wherein said cancer is pancreatic cancer, colorectal cancer, or any other primary or metastatic solid tumor type, preferably is pancreatic cancer or colorectal cancer.

Embodiment 35: A method of identifying a TCR binding to a T-cell activating antigen presented on a cell, preferably a cancer cell, of a subject, said method comprising
(A) identifying a reactive T-cell according to the method of any one of embodiments 1 to 3 and 29 to 34,
(B) providing the amino acid sequences of at least the complementarity determining regions (CDRs) of the TCR of the reactive T-cell identified in step (A); and, hereby,
(C) identifying a TCR binding to an activating antigen presented on a cell.

Embodiment 36: The method of any one of embodiments 1 to 3 and 29 to 35, wherein expression of at least one biomarker of step a) and/or the nucleic acid sequences encoding the amino acid sequences of step (B) is/are determined by single-cell sequencing, preferably by single-cell RNA sequencing.

Embodiment 37: The method of embodiment 35 or 36, wherein said method comprises further step B1) expressing a TCR comprising at least the CDRs determined in step B) in a host cell, preferably a T-cell.

Embodiment 38: The method of embodiment 37, wherein said method further comprises further step B2) determining binding of the TCR expressed in step B1) to a T-cell activating antigen, preferably complexed in a major histocompatibility complex (MHC), preferably MHC class I, molecule, preferably in a tetramer assay.

Embodiment 39: The method of embodiment 37 or 38, wherein said method further comprises step B3) determining recognition of cells presenting a T-cell activating antigen by the TCR expressed in step B1).

Embodiment 40: The method of any one of embodiments 35 to 39, wherein said method further comprises step B4) producing a soluble TCR comprising at least the CDRs determined in step B) and determining binding of said soluble TCR to a cancer cell and/or to a cancer antigen complexed in a major histocompatibility complex (MHC), preferably MHC class I, molecule.

Embodiment 41: A method of providing a T-cell recognizing a cell presenting a T-cell activating antigen, preferably a cancer cell, said method comprising
(i) identifying a TCR binding to a cell presenting a T-cell activating antigen according to the method according to any one of embodiments 35 to 40,
(ii) expressing a TCR comprising at least the complementarity determining regions (CDRs) of the TCR of step (I) in a T-cell, and, thereby,
(iii) providing a T-cell recognizing a cell presenting a T-cell activating antigen, preferably a cancer cell.

Embodiment 42: A reactive T-cell identified by the method according to any one of embodiments 1 to 3 and 29 to 34 and/or obtained or obtainable by the method according to any one of embodiments 35 to 40, preferably comprising a T-cell receptor comprising an amino acid sequence of SEQ ID NO:1 and/or SEQ ID NO:2, for use in medicine or for use in treating and/or preventing cancer in a subject.

Embodiment 43: A method of identifying at least one biomarker of reactive T-cells, comprising
(I) providing expression data of a plurality of biomarkers of T-cells in a sample of a subject,
(II) providing a clustering said plurality of T-cells based on the expression of the biomarkers of step (I);
(III) providing amino acid sequences of at least the complementarity determining regions (CDRs) of TCRes of T-cells of step (II);
(IV) determining reactivity of T-cells expressing a TCR comprising the CDRs of step (III) to cells presenting a T-cell activating antigen;
(V) repeating steps (III) and (IV) at least once for further T-cells clustering with T-cells whose TCRs are determined to be reactive to cells presenting a T-cell activating antigen in step (IV), wherein the TCRs of said further T-cells are non-identical to the TCRs of step (IV);
(VI) determining at least one cluster of step (II) comprising the highest fraction of T-cells comprising T-cell receptors recognizing cells presenting a T-cell activating antigen; and
(VII) determining at least one biomarker expressed by the highest fraction of T-cells in the cluster determined in step (VI), thereby identifying at least one biomarkers of cancer-reactive T-cells.

Embodiment 44: The subject matter of any one of embodiments 1 to 3 and 29 to 43, wherein said T-cell(s) is/are CD8+ T-cell(s) or CD4+ T-cells, preferably CD8+ T-cell(s).

Embodiment 45: The subject matter of any one of embodiments 35 to 44, wherein said TCR comprises, preferably consists of, a TCR alpha chain and a TCR beta chain.

All references cited in this specification are herewith incorporated by reference with respect to their entire disclosure content and the disclosure content specifically mentioned in this specification.

### FIGURE LEGENDS

FIG. 1: A) and B) show results of UMAP clustering of T Cells for 2 patients separately. D)-F) and Figure G)-J) show the expression of the core genes CCL3, CCL3L1, CCL4 and CCL4L2 in the clustered cells, respectively, for patient 1 (D)-F)) and Patient 2 (G)-J)), (K) shows the expression of the core gene CXCL13 in Patient 2.

FIG. 2: shows the clusters of cancer-reactive T-cells defined based on the expression of core genes CCL3, CCL3L1, CCL4 and CCL4L2 for Patient 1 (A)) and Patient 2 (B) whereas C) shows the cluster of cancer-reactive T-cells defined based on the expression core gene CXCL13 in Patient 2)

FIG. 3: A) shows the distribution of selected TCR clones (X-axis) in transcriptomic clusters (Y-axis) for Patient 1. B) shows the clustering of TCR based on the TCR fraction in the reactive cluster and C) shows the TCR testing result based on FACS based assay.

FIG. 4: A) shows the distribution of selected TCR clones (X-axis) in transcriptomic clusters (Y-axis) for Patient 2. B) shows the clustering of TCR based on the TCR fraction in the reactive cluster and C) shows the TCR testing result based on NFAT reporter assay: Co-culture of TCR transgenic Jurkat cells with peptide-loaded autologous PBMCs confirms that TCR4 recognizes the IDH1.R132H mutant epitope expressed by the tumor. Data depicted as mean+SD of 3 technical replicates. Representative of 3 independent experiments. CD3+CD28 stimulation represents the maximum possible activation of T cells. MOG is negative control peptide not bound by either TCR in the assay.

FIG. 5: shows results of UMAP clustering of T Cells for 9 pancreatic ductal adenocarcinoma (PDAC) samples from 9 patients with primary respectable PDAC consisting of 17.855 T-cells.

FIG. 6: A) shows the distribution of the T-cells expressing tumor-reactive T-cell receptors (TCRs) on the UMAP of T Cells for 9 pancreatic ductal adenocarcinoma (PDAC) samples. B) shows the distribution of the T-cells expressing tumor non-reactive TCRs on the UMAP of T cells from 9 pancreatic ductal adenocarcinoma (PDAC) samples. In total 106 TCRs representing unique T-cell clonotypes were tested, of which 53 were found to be tumor-reactive and 53 tumor non-reactive. Each TCR was tested in at least 3 independent experiments with the same outcome.

FIG. 7: A) shows a representative example of an in vitro experiment assessing the reactivity of TCRs isolated from human pancreatic ductal adenocarcinoma (PDAC) samples by means of single cell sequencing. Primary human T-cells were transduced with the indicated TCR by means of RNA gene transfection, followed by incubation with or without tumor cells and determination of T-cell reactivity by means of intracellular flow cytometry staining for the T-cell activation marker TNF-alpha (TNFa). The TCRs indicated were all derived from one of the 9 PDAC tumors and were tested against an autologous tumor cell line derived from the same PDAC tumor sample. As indicated, the TCRs were reproducibly found to be tumor non-reactive (TNR) or tumor-reactive (TR). Mock-transfected T-cells were used as a negative control. Gene constructs encoding the transfected TCRs comprise the variable regions of the TCR alpha and beta chains of interest in combination with the constant regions of mouse TCR alpha and beta chains. This promotes correct pairing of the gene transduced TCR genes, and enables detection of transgene-encoded TCR expression by means of an antibody against the mouse TCR constant domain (mTCRb). As is demonstrated by the data, tumor-reactivity is only seen for T-cells expressing the transgene-encoded TCRs, more specifically expressing the tumor-reactive (TR) TCRs. B) shows the same experiment as above with the only difference that T-cell reactivity is assessed by staining for the T-cell activation marker CD107a.

FIG. 8: shows the distribution on the UMAP from 9 pancreatic ductal adenocarcinoma (PDAC) samples of T-cells predicted to express tumor-reactive TCRs by means of the genes comprised in Gene Set 1. The large UMAP plot shows the result of the prediction using all five genes comprised within Gene Set 1 (Signature 1=SIGN1). The small UMAP plots show the result of the prediction using each of the five single genes. Comparison with the UMAP plot in FIG. 6A reveals that of these five genes, CXCL13 and CCL3 are the best biomarkers for T-cells expressing tumor-reactive TCRs.

FIG. 9: shows the distribution on the UMAP of T Cells from 9 pancreatic ductal adenocarcinoma (PDAC) samples of T-cells predicted to express tumor-reactive TCRs by means of the genes CXCL13 and CCL3 only (Signature 2=SIGN2).

FIG. 10: A) shows the distribution on the UMAP from 9 pancreatic ductal adenocarcinoma (PDAC) samples of T-cells predicted to express tumor-reactive TCRs by means of the genes CXCL13 and CCL3 in combination with the eleven genes comprised by Gene Set 2 (together: Signature 3=SIGN3). The large UMAP plot shows the result of the prediction of tumor-reactive (TR) T-cells using all thirteen genes (SIGN3). B) shows small UMAP plots of the prediction of tumor-reactive (TR) T-cells using each of the seven single genes that were selected as biomarkers within SIGN3 for predicting T-cells expressing tumor-reactive (TR) TCRs. C) shows small UMAP plots of the prediction of tumor non-reactive (TNR) T-cells using each of the six single genes that were selected within SIGN3 as biomarkers for predicting T-cells expressing tumor non-reactive (TNR) TCRs

FIG. 11: A) shows the distribution on the UMAP from 9 pancreatic ductal adenocarcinoma (PDAC) samples of T-cells predicted to express tumor-reactive TCRs by means of the genes CXCL13 and CCL3 in combination with the genes comprised by Gene Set 2 as well as with the genes comprised by Gene Set 3 (together: Signature 4=SIGN4). The large UMAP plot shows the result of the prediction of tumor-reactive (TR) T-cells using all ninety genes (SIGN4). B) shows the UMAP plots of the prediction of tumor-reactive (TR) T-cells using all 70 genes that were selected as biomarkers within SIGN4 for predicting T-cells expressing tumor-reactive (TR) TCRs. C) shows small UMAP plots of the prediction of tumor non-reactive (TNR) T-cells using all 20 genes that were selected within SIGN4 as biomarkers for predicting T-cells expressing tumor non-reactive (NTR) TCRs.

FIG. 12: A) shows the accuracy of prediction of each of the 4 aforementioned gene signatures (SIGN1-4) using the single T-cell data set from the 9 pancreatic ductal adenocarcinoma (PDAC) samples, using the actual reactivity data from the 106 tested TCRs (FIG. 6) as a reference Ucell scores for all cells of the Seurat objects were determined by using the AddModuleScore_Ucell function. The output data was filtered to contain only cells of functionally tested clonotypes that were either tumor reactive (TR) or tumor non-reactive (NTR) (FIG. 6). All clonotypes scoring higher than 0.02, were noted as predicted to be reactive. All clonotypes scoring 0.02 or less, were noted as predicted to be non-reactive. On this basis we calculated the percentage of correct and false predictions for each signature: Correct=(True R+True NR)/(Predicted R/Predicted NR); False R=True R/(Predicted R/Predicted NR); False NR=True NR/(Predicted R/Predicted NR). As shown, SIGN3 encompassing CXCL-13, CCL-3 and the 11 genes comprised in Gene Set 1 provides the most accurate result, in that the correct prediction rate is 92.45%, while the false negative rate (false NR) is 7.55% and the false reactive (false R) rate is 0%. B) shows the result of similar analyses using SIGN3 in which one or two genes as indicated are omitted. Each of the 13 genes comprised within SIGN3 contributes to the quality of the TCR prediction, in that the False NR and/or False R rate increases in the case one of the genes is omitted. In the case of the genes IL7R and GPR183, omission of either one of these genes does not affect the quality of the prediction. However, omission of both genes does decrease the accuracy of the prediction, indicating that these genes, while being interchangeable, both contribute to the quality of the prediction.

FIG. 13: A) shows the distribution on the UMAP from 9 pancreatic ductal adenocarcinoma (PDAC) samples of T-cells predicted to express tumor-reactive TCRs by means of either the genes CXCL13 and CCL3 in combination with the genes comprised by Gene Set 2 (together: SIGN3), or by the 85-gene set comprising all genes for either CD8-positive or unspecified T-cells, specifically ACP5, AF243829.4, AFAP1IL2, AHI1, ALOX5AP, APOBEC3C, APOBEC3G, ARHGAP9, ASB2, CARD16, CCL3, CCL4, CCL4L2, CD3G, CD74, CD8A, CD8B, CLIC3, CST7, CTSW, CXCL13, CXCR6, ENTPD1, GALNT2, GNLY, GZMA, GZMB, GZMH, GZMK, HAVCR2, HCST, HLA-DMA, HLA-DPA1, HLA-DPB1, HLA-DR, HLA-DRA, HLA-DRB1, HLA-DRB5, HMGN3, HMOX1, IFNG, ITGAE, ITGAL, ITM2A, JAML, KLRB1, LINC01871, LYST, MIR155HG, MPST, NAP1L4, NELL2, NKG7, NSMCE1, ORMDL3, PD-1, PDLIM4, PLEKHF1, PPP1R16B, PRF1, PTMS, RAB27A, RARRES3, RBPJ, RGS1, SLF1, SMC4, TIGIT, TNFRSF7, TNFRSF9, VCAM1, ANXA1, CCR7, CD45RA, EEF1B2, EMP3, FCGR3A, IL7R, LGALS3, LTB, LYAR, RGCC, RPL36A, S100A10 and SELL, as described in NIH patent application WO 2021/188954 (NIH). B) shows the accuracy of prediction of each of the 2 aforementioned signatures using the single T-cell data set from the 9 pancreatic ductal adenocarcinoma (PDAC) samples, using the actual reactivity data from the 106 tested TCRs (FIG. 6) as a reference and the methodology as described in the legend to the previous figure. C) shows the prediction scores obtained for each of the 106 T-cell clonotypes with known TCR reactivity (R=tumor-reactive; TNR=tumor non-reactive). Ucell scores for all cells of the Seurat objects were determined by using the AddModuleScore_Ucell function as described for FIG. 12. Based on the highest score per clonotype of the tumor non-reactive (TNR) (0.019), the cut of value for the tumor-reactivity 9TR) prediction score was set to 0.02 to validate and compare the predictions accuracies of various gene signatures. The mean score per clonotype was calculated and plotted separately for TR and TNR clonotypes.

FIG. 14: A) upper panel shows the distribution of the T-cells expressing tumor-reactive (TR) T-cell receptors (TCRs) on the UMAP of T Cells from lung cancer patient MD01-004, as published by Caushi et. al. (2021), Nature 596(7870):126. The lower panel shows the distribution of the T-cells expressing tumor non-reactive (TNR) TCRs on the same UMAP. In total 19 TCRs representing unique T-cell clonotypes were tested, of which 14 were found to be tumor-reactive (TR) and 5 tumor non-reactive (TNR). B) shows the distribution on the UMAP of lung cancer patient MD01-004 of T-cells predicted to express tumor-reactive TCRs by means of either the genes CXCL13 and CCL3 in combination with the genes comprised by Gene Set 2 (together: SIGN3), or by the 85-gene set comprising all genes for either CD8-positive or unspecified T-cells (see legend to FIG. 13 for specific gene names), as described in NUH patent application WO 2021/188954 (NIH).

The following Examples shall merely illustrate the invention. They shall not be construed, whatsoever, to limit the scope of the invention.

### EXAMPLE 1: SINGLE CELL LIBRARY PREPARATION

Single cell suspension of tumor was FACS-sorted for CD45⁺CD3⁺ population to enrich for T Cells. Single cell library construction of sorted T Cells was performed using Chromium Single Cell Immune Profiling Kit (10× Chromium) according to the manufacturer's protocol. The constructed scVDJ and scRNA library were then sequenced on Hiseq2500 Rapid/Nextseq550 and Hiseq4000 (Illumina) respectively.

### EXAMPLE 2: SINGLE CELL RNA ANALYSIS

Sequencing Raw data was processed with cellranger pipeline (v3.1.0) with corresponding GRCh38 genome assembly with default settings to generate gene expression matrices. Matrices were imported into R and analyzed using the Seurat package. For quality control, outliers were removed based on UMI, the number of genes and the percentage of mitochondrial gene expression. Then, gene expression was transformed and normalized and VDJ genes were then subsequently removed from the variable genes. Highly variable genes were selected based on Principal Component Analysis and the number of components was selected based on inflection point in the elbow plot. Cells were then clustered using unsupervised graph-based clustering method and UMAP were plotted for visualization. Differential gene expression analysis was done using MAST and the upregulated genes were used to define each cluster.

The scVDJ data was processed similarly using the cellranger pipeline with default settings. The T cell receptor data was then mapped onto gene expression data to determine the distribution of individual TCR clones transcriptomically. K-mean clustering was done to cluster the TCR based on their distribution in transcriptomic clusters.

### EXAMPLE 3: CLONING

For cloning the TCRs, synthetic alpha and beta VDJ fragments of the variable region of the TCR were obtained from Twist Biosciences. The TCR variable fragments were inserted into an S/MAR sequence-bearing expression vector (pSMARTer) that allows extrachromosomal replication of the vector in eukaryotic cells using a single-step Bsa-I mediated Golden Gate reaction. The expression vector was designed to harbor murine alpha and beta constant TCR regions and a p2a self-cleaving peptide linker to facilitate production of separate alpha and beta polypeptide chains of the TCR. The vector was subsequently transformed into NEB5-alpha competent E. coli (NEB); colonies screened for transgene by antibiotic resistance; and endotoxin-free plasmid prepared using NucleoBond Extra Maxi EF kit (Macherey-Nagel) for transfection. In some experiments, the TCR variable fragments were inserted into a pcDNA3.1 plasmid backbone towards in vitro transcription and RNA transfection.

### EXAMPLE 4: NFAT ASSAY

The cloned TCR expression vector and a nano-luciferase-based NFAT reporter vector (pDONR, with 4×NFAT-response elements) were transfected into Jurkat Δ76 cells using electroporation (Neon Transfection system, ThermoFisher Scientific). In brief, 2×10⁶ cells were used per electroporation with Neon 100 μl tips (8 μg TCR expression vector+5 μg NFAT reporter vector). Cells were harvested and washed based on manufacturer's protocol, then electroporated with 1325V, 10 ms, 3 pulses and transferred to antibiotic-free RPM1 1640 medium containing 10% FCS. Patient-autologous PBMCs were used as antigen presenting cells (APCs) and thawed 24 h before co-culture in X-VIVO 15 medium (Lonza) containing 50 U/ml Benzonase (Sigma-Aldrich), and rested for 6-8 h before seeding into 96-well white-opaque tissue culture treated plates (Falcon) at 1.5×105 cells per well. Cells were loaded with peptides at a final concentration of 10 μg/ml in a total volume of 150 μl for 16 h. Peptides utilized were human IDHIR132H peptides (p123-142), MOG (p35-55) at equal concentrations and PBS+10% DMSO (vehicle) at equal volume as negative controls. 48 h post electroporation, TCR-transgenic Jurkat Δ76 cells were harvested and co-cultured with peptide-loaded PBMC for 6 h at a 1:1 ratio. Human T-cell TransAct beads (Miltenyi) were used as positive control. A publicly known TCR against InfluenzaHA (p307-319) was used as an assay reference. Nano-luciferase induction indicating TCR activation was assayed using Nano-Glo Luciferase assay system (Promega) according to manufacturer's protocol and signal was detected on PHERAstar FS plate reader (BMG Labtech).

### EXAMPLE 5: FACS-BASED ASSAY

Cloning was done as described above with the addition of T7 promoter by PCR. The PCR product was then purified using DNA Clean & Concentrator-5 (Zymo Research) and used as a template for in vitro transcription using Cellscript kit according to the manufacturer's protocol. The concentration and integrity of RNA was assessed by Nanodrop and Bioanalyzer respectively. The RNA was then electroporated into expanded autologous PBMC using the Lonza 4D nucleofactor device. After electroporation, cells were incubated at room temperature for 10 minutes before plating into 48-well plate containing 1 mL of media (TexMACS+2% AB) and allowed to rest overnight. Prior to incubation, 150 k cells were stained with CD3, CD4, CD8a, mTCRP to use as a control. The rest of the electroporated cells were then co-incubated with target cells (tumour cell line/patient derived xenograft) for 5 hours, with the addition of Golgistop and Golgiplug after 1 hour. After co-incubation, cells were stained for dead cell biomarker, CD3, CD4, CD8a, mTCRβ, TNFα and IFNγ, and then measured using FACSLyric (BD Biosciences). In some experiments, cells were stained for dead cell biomarker, CD3, CD4, CD8a, mTCRβ, TNFα and CD107a, and then measured using FACSLyric. The analysis was done using FlowJo.

### EXAMPLE 6: RESULTS-1

### 6.1 Clustering of T Cells Based on Gene Expression

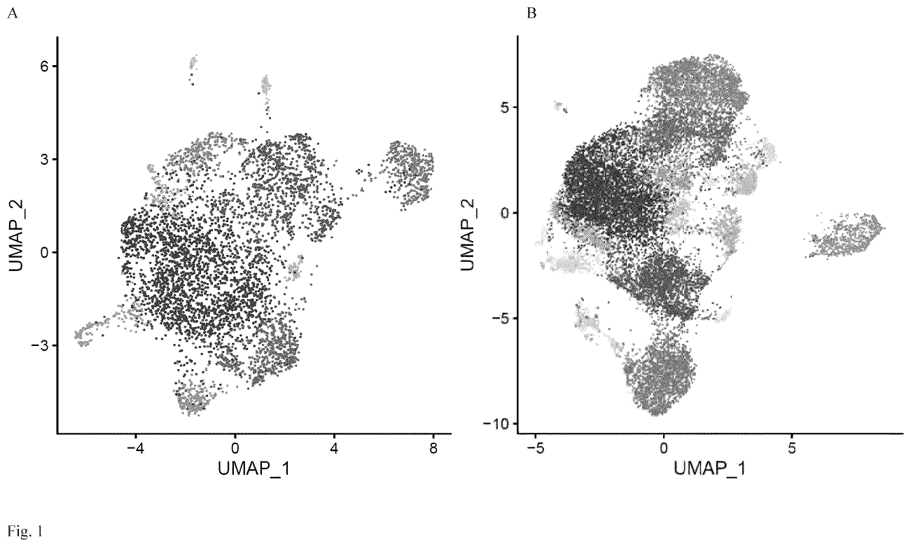

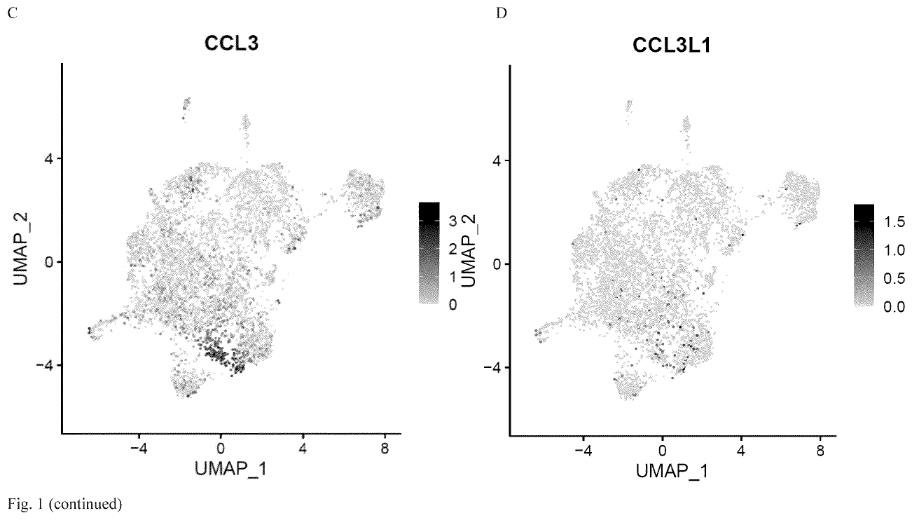

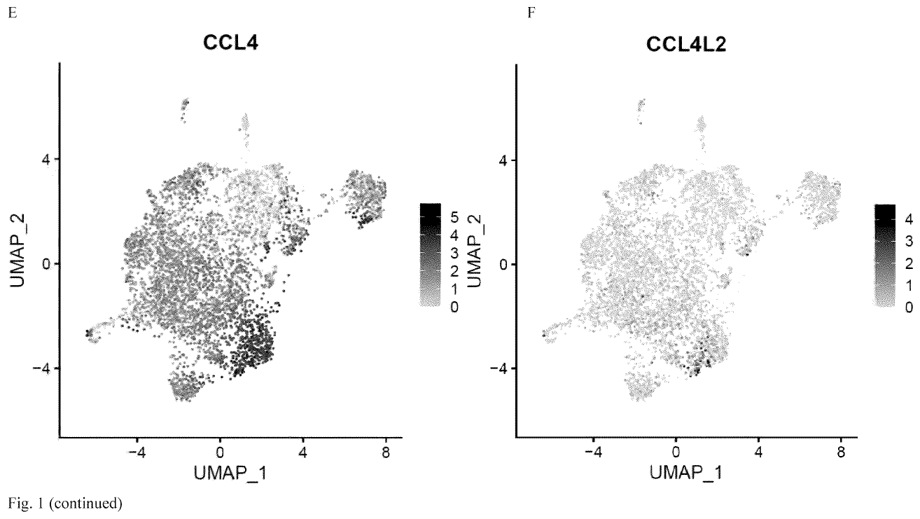

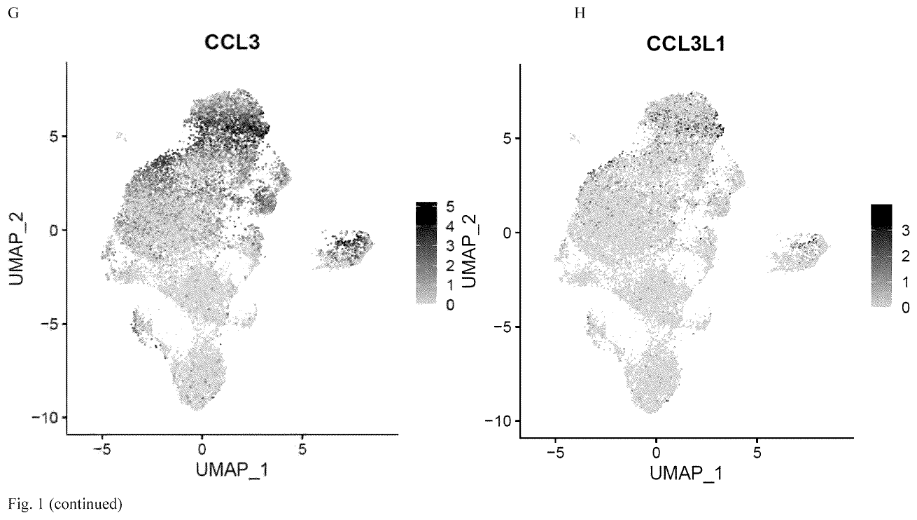

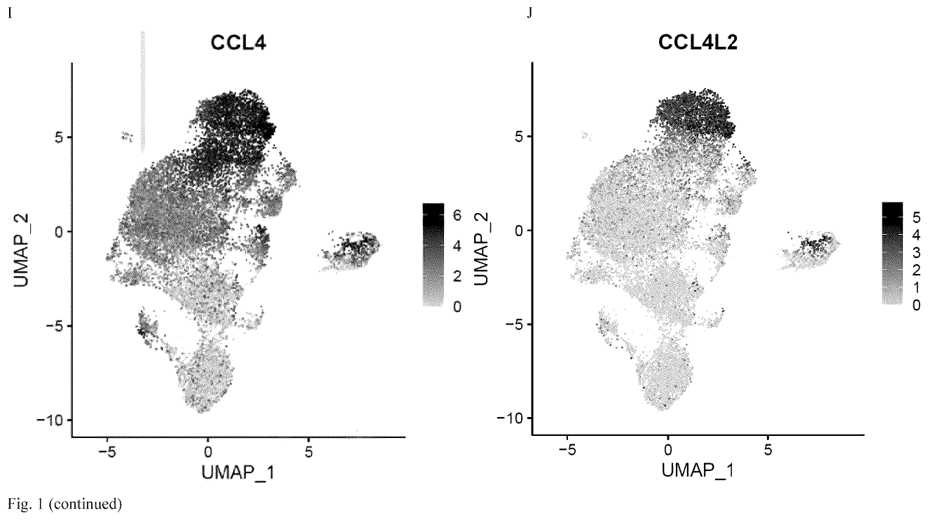

Single cell RNA-seq dataset was normalized, transformed and clustered using graph-based unsupervised clustering. 2 selected patients data are shown here. 15 clusters and 16 clusters were identified from Patient 1 (FIG. 1A) and Patient 2 (FIG. 1), respectively.

### 6.2 Expression of Signature Genes

Differential gene expression was done using MAST and the upregulated gene expression for each cluster was found. In multiple patients, we have identified a cluster that expressed the signature genes CCL3, CCL3L1, CCL4 and CCL4L2. FIG. 1D-1F and FIG. 1G-1J show the expression of the signature genes of 2 selected patients. Another signature gene CXCL13 expression is also shown in a selected patient (FIG. 1K).

### 6.3 Defining Reactive Clusters Based on Signature Genes

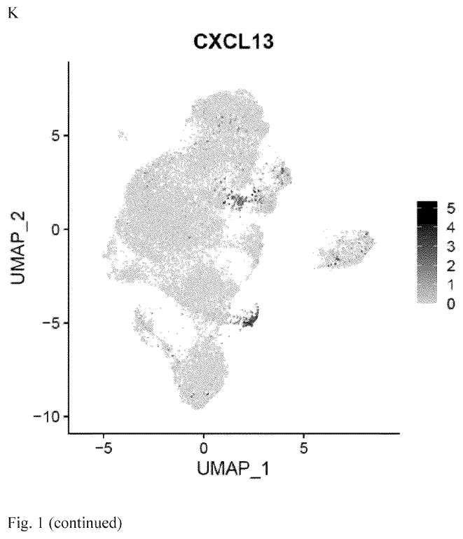

Based on the expression of signature genes CCL3, CCL3L1, CCL4 and CCL4L2, a reactive cluster was defined (FIGS. 2A and 2B). The reactive cluster defined based on the expression of the signature gene CXCL13 was depicted in FIG. 2C.

### 6.4 CCL3/CCL3L1/CCL4/CCL4L2 Signature

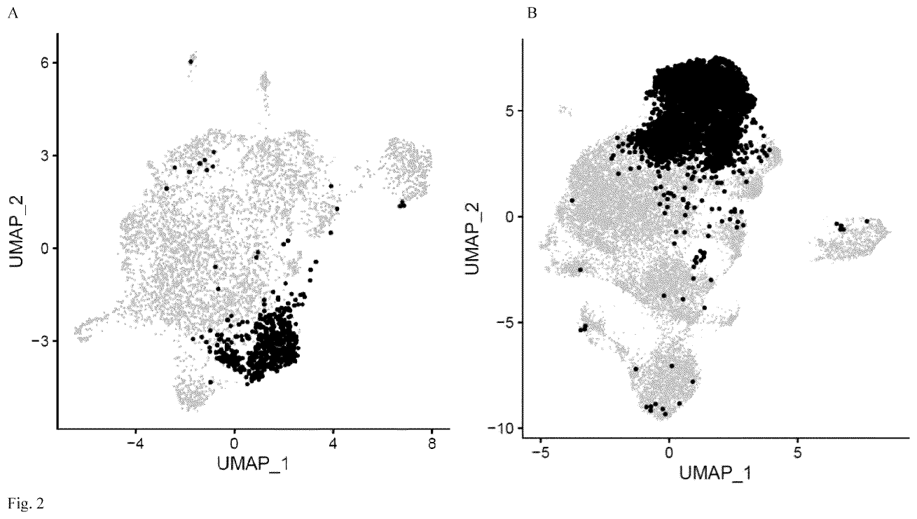

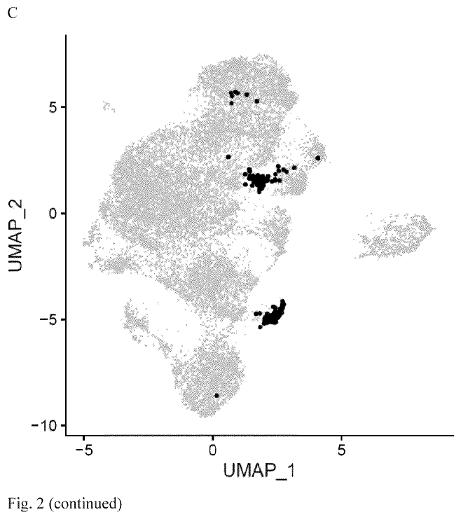

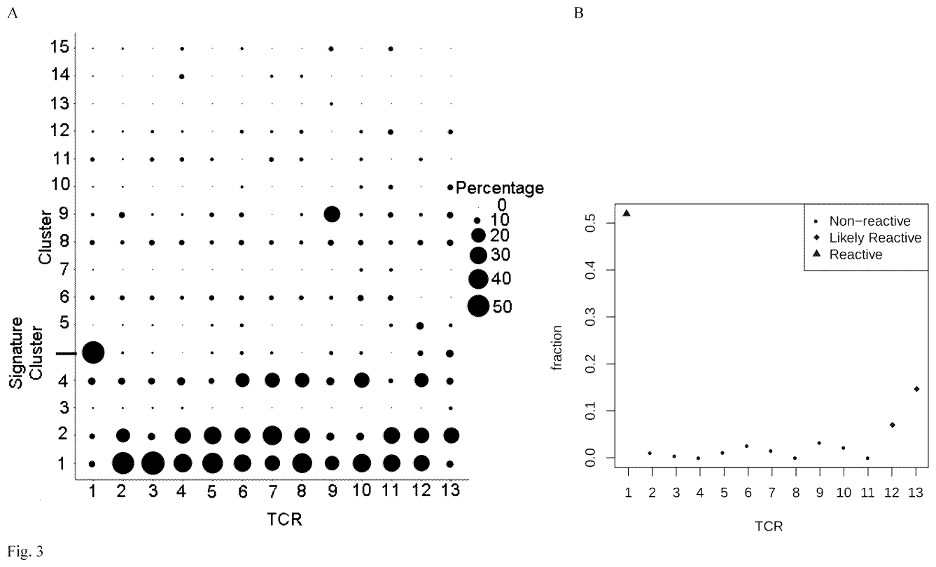

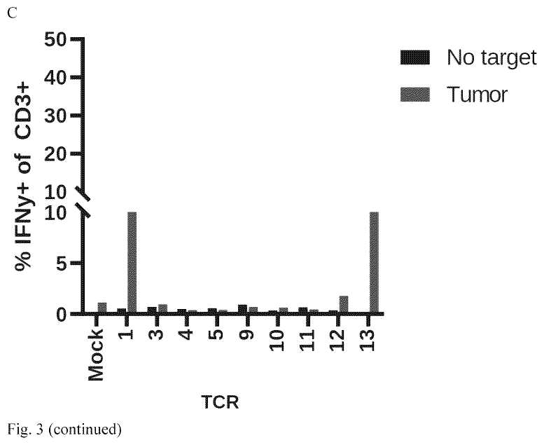

FIG. 3A depicts the top 13 highest frequency TCR clonotypes in Patient 1. As described previously, cluster 4 which expressed the signature genes was defined as the signature cluster. From the distribution, we can clearly see that TCR1, TCR12 and TCR13 have higher distribution of T Cell in signature cluster.

FIG. 3B shows the k-mean clustering result based on the fraction of T Cells in the signature cluster. 3 clusters were found based on this clustering result. Cluster with high fraction of T Cells in the signature cluster should be reactive, cluster with moderate fraction of T Cells should be likely-reactive and the cluster with lowest fraction of T Cells in the signature cluster should be non-reactive. Thus, TCR1 was predicted to be reactive, and TCR12 and TCR13 was predicted to be likely reactive while the other TCR are non-reactive. TCRs were then cloned to test the tumour reactivity of these TCR and to corroborate the TCR prediction based on signature genes.

FIG. 3C shows the result of FACS-based TCR testing. As predicted by the signature genes, only TCR1, TCR13 and possibly TCR12 secrete IFNγ upon coculture with the corresponding patient's tumour cells, thus showing TCR1 and TCR13 are indeed reactive to cancer cells with TCR12 being possibly reactive.

### 6.5 CXCL13 Signature

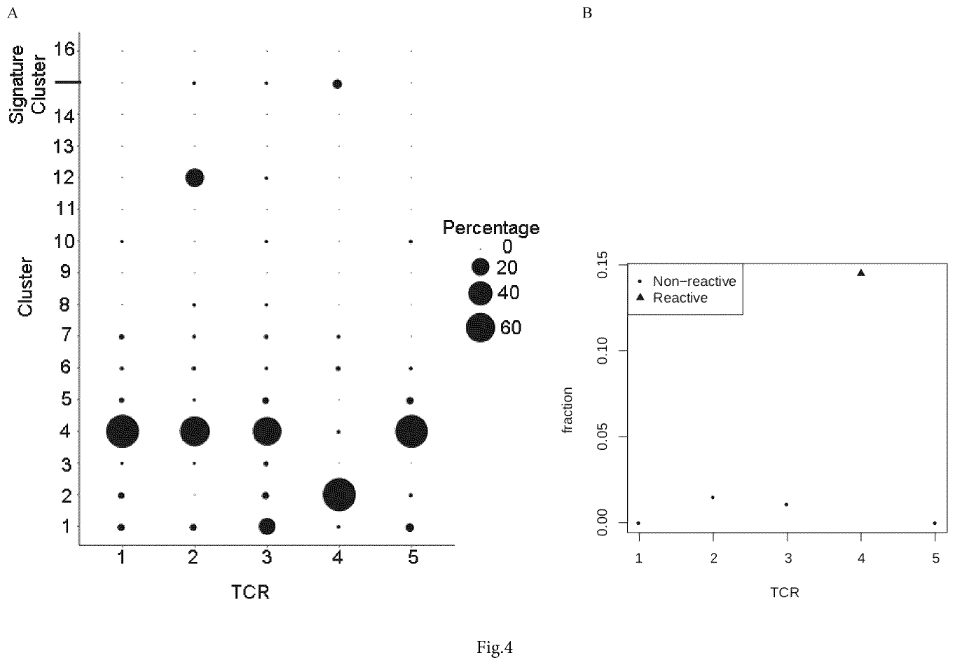

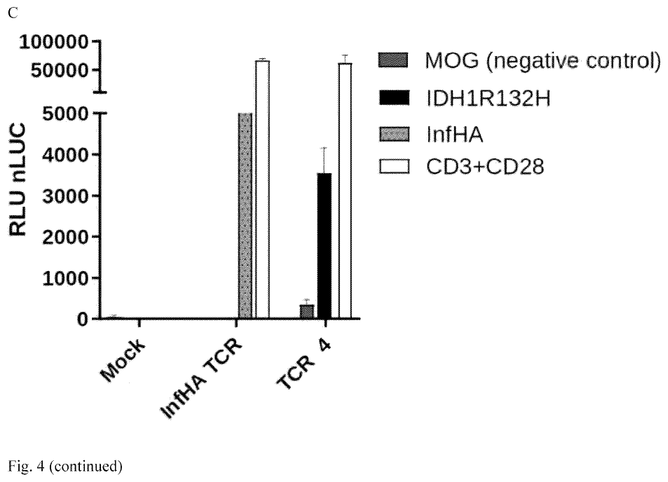

FIG. 4A illustrates the top 5 highest frequency CD4 TCR in Patient 2. From the distribution, it is clear that TCR4 was the only TCR to have higher distribution in the signature cluster. A further k-mean clustering (FIG. 4B) also found that there were 2 clusters based on the T Cell fraction in this signature cluster. The cluster with the higher fraction, which consist of only TCR4, was then predicted to be reactive. This TCR was then cloned and tested with NFAT assay. TCR4 which was predicted to be tumour reactive by gene signature is indeed reactive (FIG. 4C) when coculture with peptide-loaded PBMC. The exact sequence for this TCR4 is as shown in SEQ ID NOs:1 and 2.

### EXAMPLE 7: RESULTS-2

7.a Clustering of T-Cells Isolated from Human Pancreatic Cancer Samples Based on Gene Expression

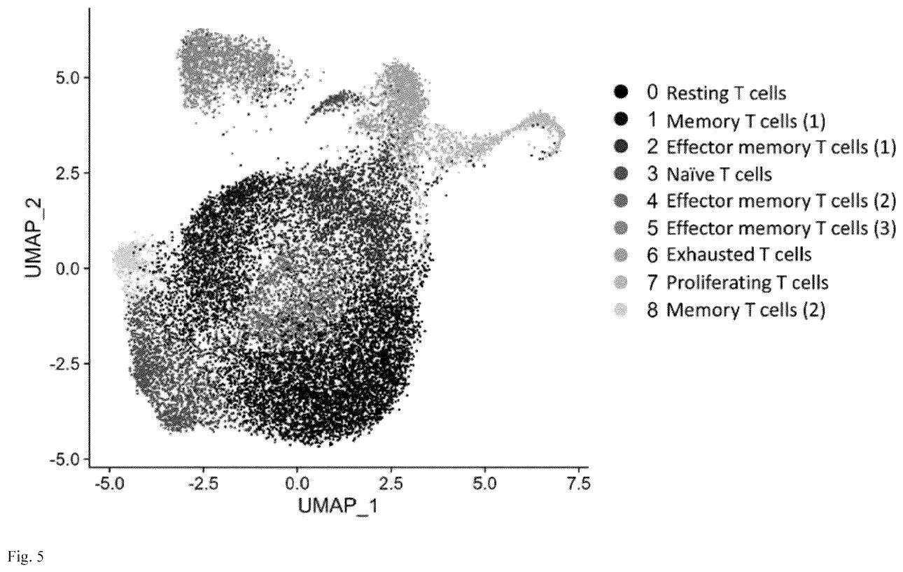

Single cell RNA-seq dataset was normalized, transformed and clustered using graph-based unsupervised clustering. Combined data from 9 patients data are shown here revealing 9 clusters (FIG. 5).

7.b Association of Functional TCR Reactivity with Gene Expression

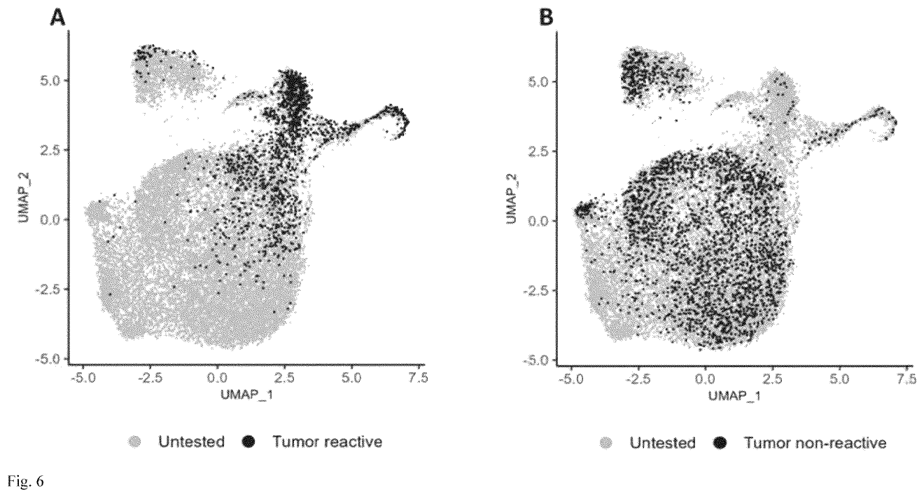

Distribution of the T-cells expressing tumor-reactive T-cell receptors (TCRs) (FIG. 6A) and tumor non-reactive TCRs (FIG. 6B) on the UMAP of T Cells from 9 pancreatic ductal adenocarcinoma (PDAC) samples. In total 106 TCRs representing unique T-cell clonotypes were tested, of which 53 were found to be tumor-reactive and 53 tumor non-reactive.

### 7.c Analysis of In Vitro TCR Reactivity Against Autologous Tumor Cells

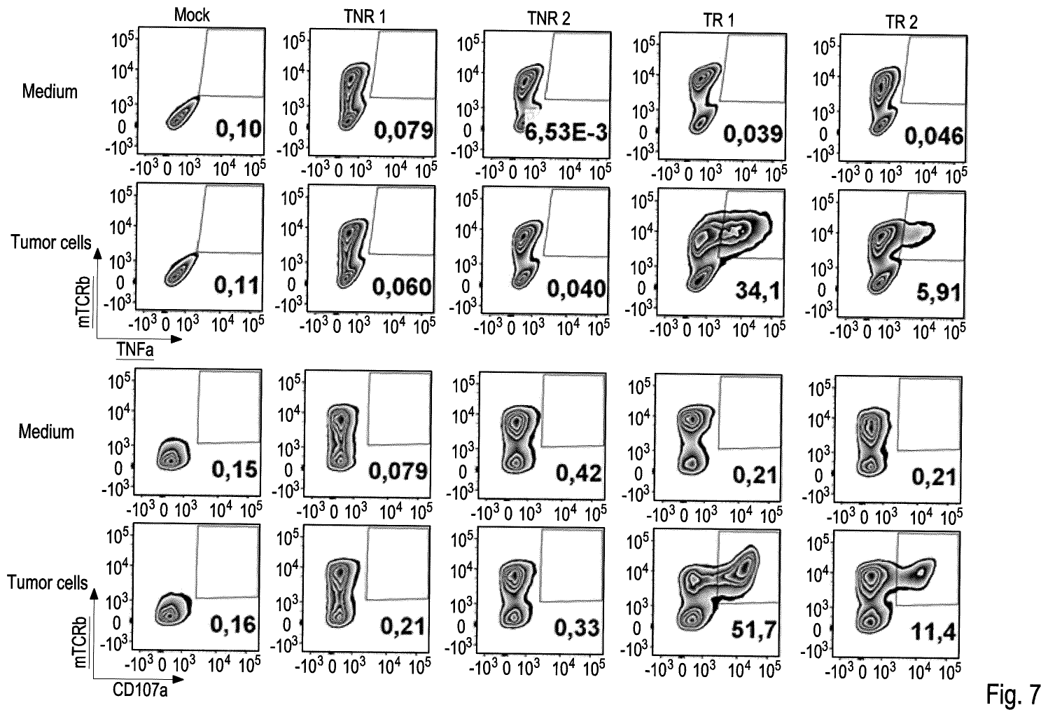

TCRs isolated from human pancreatic ductal adenocarcinoma (PDAC) samples by means of single cell sequencing were tested for tumor-reactivity as follows. Primary human T-cells were transduced with the indicated TCR by means of RNA gene transfection, following incubation with or without tumor cells and determination of T-cell reactivity by means of intracellular flow cytometry staining for the T-cell activation marker TNF-alpha (TNFa; FIG. 7A) or CD107a (FIG. 7B). The TCRs indicated were all derived from one of the 9 PDAC tumors and were tested against an autologous tumor cell line derived from the same PDAC tumor sample. As indicated, the TCRs were reproducibly found to be tumor non-reactive (TNR) or tumor-reactive (TR). Mock-transfected T-cells were used as a negative control.

7.d Identification of Genes Associated with T-Cells Expressing Tumor-Reactive TCRs Genes identified as associated with T-cells expressing tumor-reactive (TR) TCRs are shown in Table 8. Genes were defined through differential-expression tests between tumor-reactive clusters (cluster 6) and tumor-non-reactive clusters (cluster 1, 3, and 8) using FindMarkers function in Seurat with logistic regression (LR) test. A difference of 0.8-fold (log-scale) or greater between the two groups of cells was used as cut-off values. All listed genes are highly expressed in cluster 6.
7.e Identification of Genes Associated with T-Cells Expressing Non-Tumor-Reactive TCRs

Genes identified as associated with T-cells expressing tumor non-reactive (TNR) TCRs are shown in Table 8. Genes were defined through differential-expression tests between tumor-reactive clusters (cluster 6) and tumor-non-reactive clusters (cluster 1, 3, and 8) using FindMarkers function in Seurat with logistic regression (LR) test. A difference of 0.8-fold (log-scale) or greater between the two groups of cells was used as cut-off values. All listed genes are highly expressed in clusters 1, 3 and 8.

7.f Analysis of PDAC Single T-Cell Sequencing Data Set with CCL3L1, CCL4, CCL4L2, CCL3, and CXCL13 Gene Signature Towards Identification of T-Cells Expression Tumor-Reactive TCRs

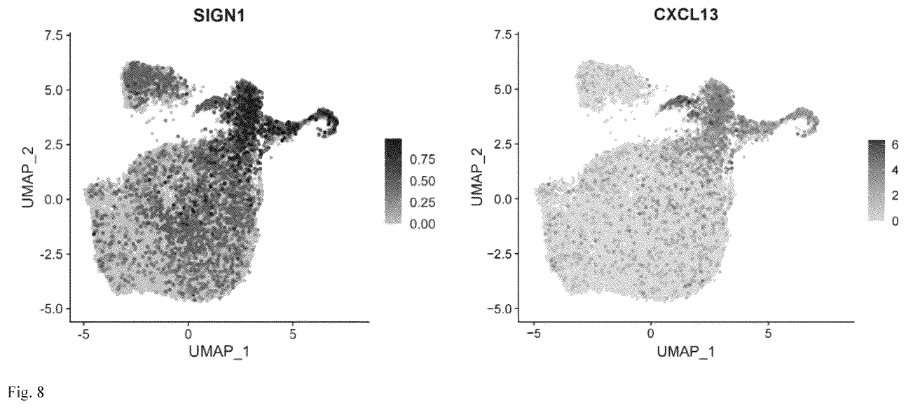

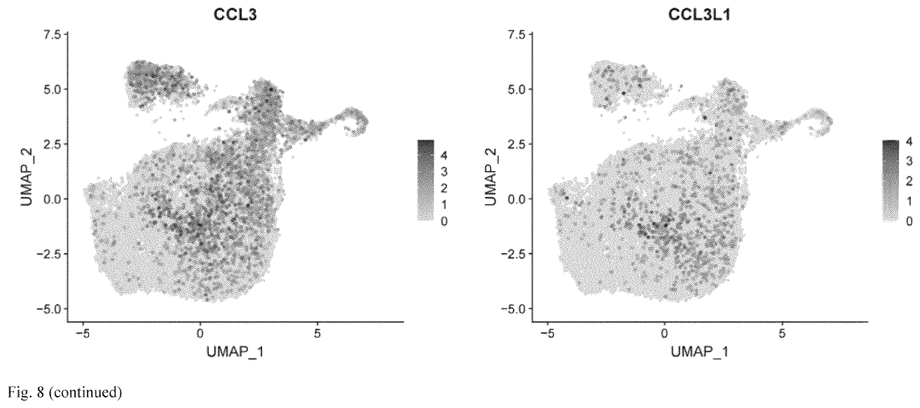

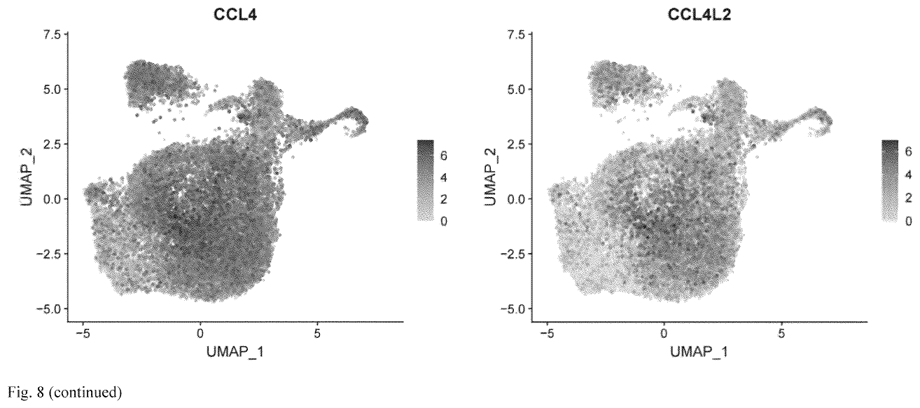

FIG. 8 shows the distribution on the UMAP from 9 pancreatic ductal adenocarcinoma (PDAC) samples of T-cells predicted to express tumor-reactive TCRs by means of the genes comprised in Gene Set 1 (CCL3L1, CCL4, CCL4L2, CCL3, and CXCL13). The large UMAP plot shows the result of the prediction using all five genes comprised within Gene Set 1 (Signature 1=SIGN1). The small UMAP plots show the result of the prediction using each of the five single genes. Comparison with the UMAP plot in FIG. 6A reveals that of these five genes, CXCL13 and CCL3 are the best biomarkers for T-cells expressing tumor-reactive TCRs.

7.g Analysis of PDAC Single T-Cell Sequencing Data with CCL3, and CXCL13 Gene Signature Towards Identification of T-Cells Expression Tumor-Reactive TCRs

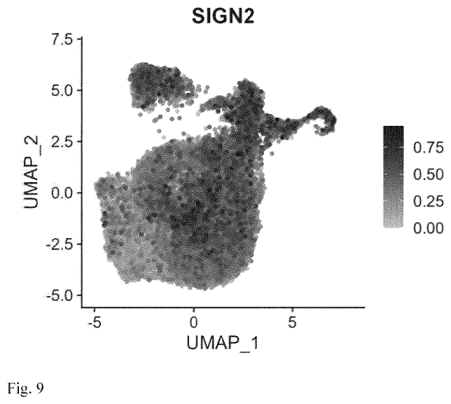

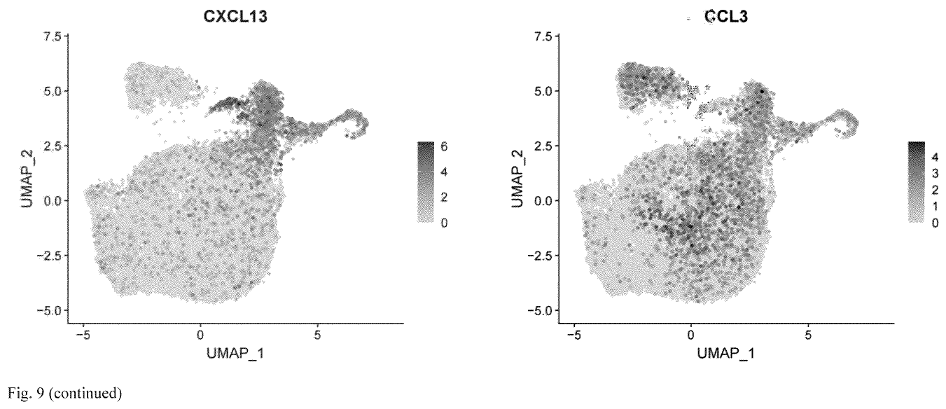

FIG. 9 shows the distribution on the UMAP from 9 pancreatic ductal adenocarcinoma (PDAC) samples of T-cells predicted to express tumor-reactive TCRs by means of the genes CXCL13 and CCL3 only (Signature 2=SIGN2).

7.h Analysis of PDAC Single T-Cell Sequencing Data with the 13-Gene Signature (SIGN3) Towards Identification of T-Cells Expression Tumor-Reactive TCRs

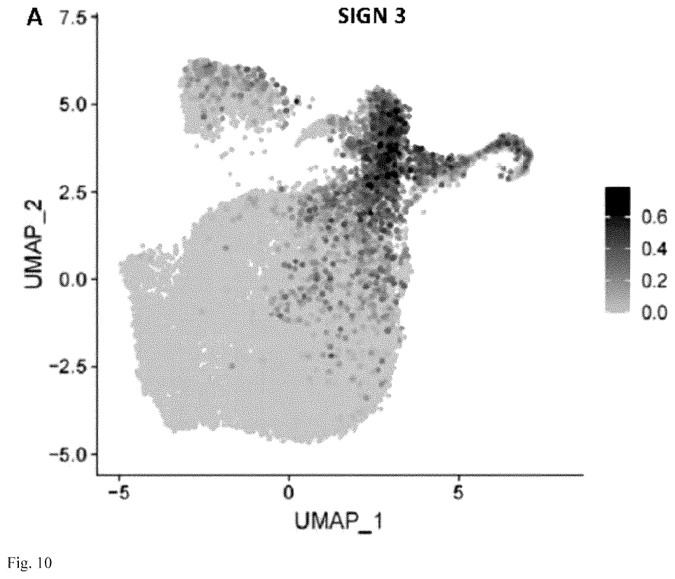

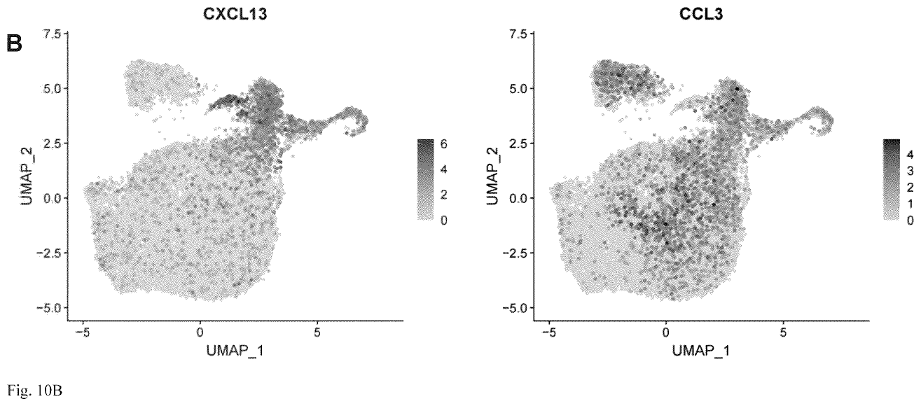

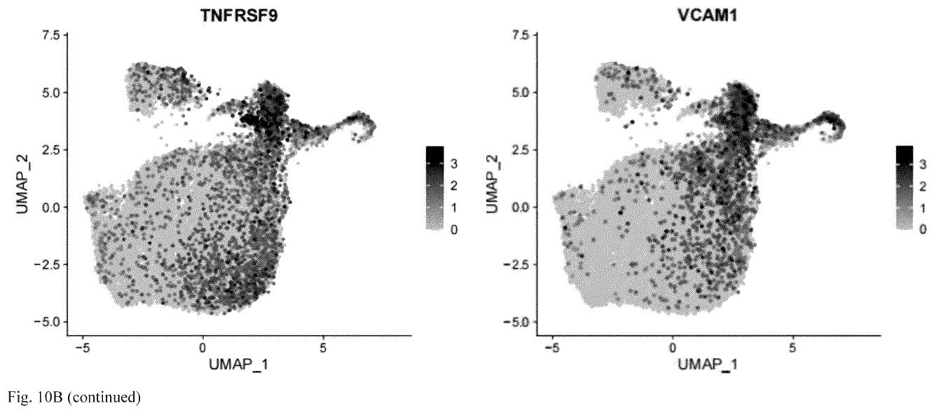

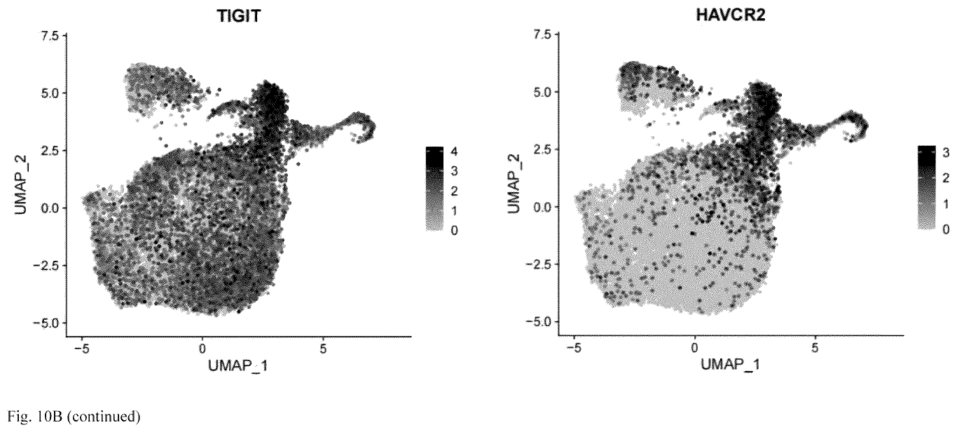

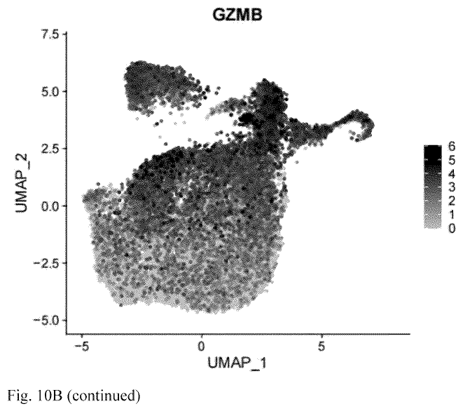

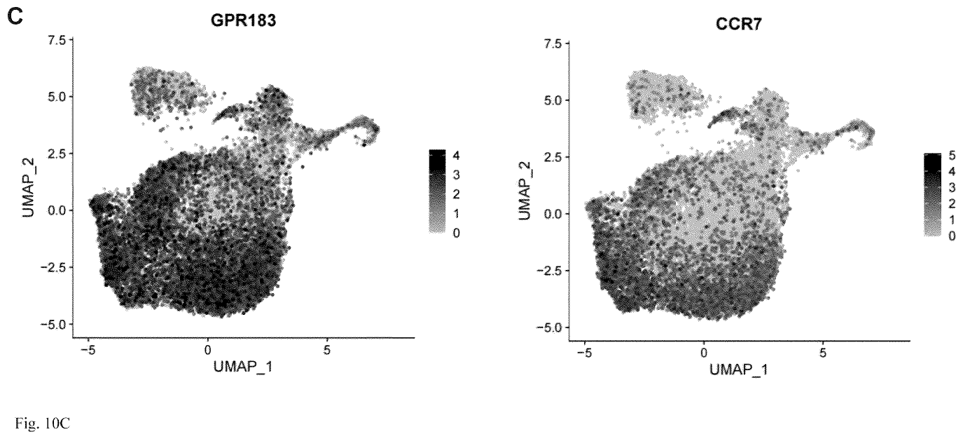

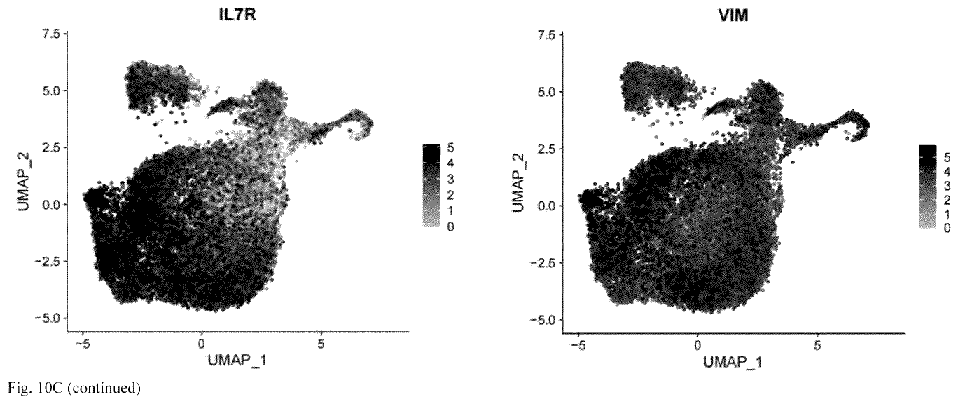

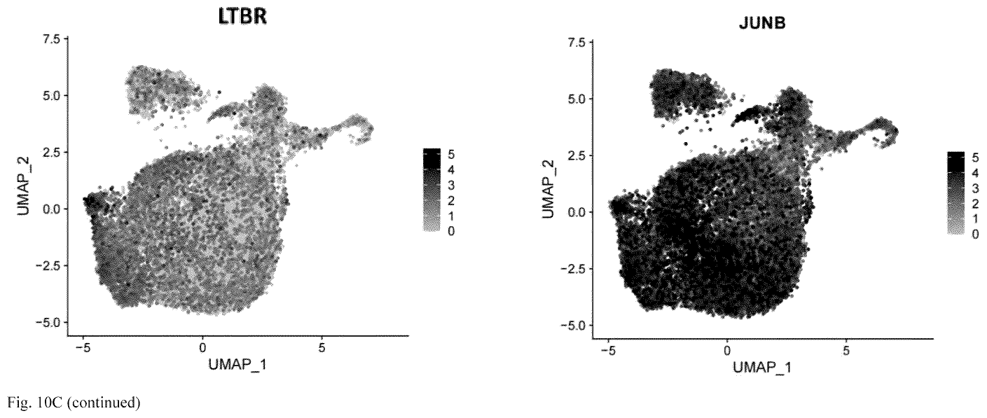

FIG. 10A shows the distribution on the UMAP from 9 pancreatic ductal adenocarcinoma (PDAC) samples of T-cells predicted to express tumor-reactive TCRs by means of the genes CXCL13 and CCL3 in combination with the eleven genes comprised by Gene Set 2 (together: Signature 3=SIGN3). The small UMAP plots in FIG. 10B show the prediction of tumor-reactive (TR) T-cells using each of the seven single genes that were selected as biomarkers within SIGN3 for predicting T-cells expressing tumor-reactive (TR) TCRs. The small UMAP plots in FIG. 10C show the prediction of tumor on-reactive (TNR) T-cells using each of the six single genes that were selected within SIGN3 as biomarkers for predicting T-cells expressing tumor non-reactive (TNR) TCRs

7.i Analysis of PDAC Single T-Cell Sequencing Data with the 90-Gene Signature (SIGN4) Towards Identification of T-Cells Expression Tumor-Reactive TCRs

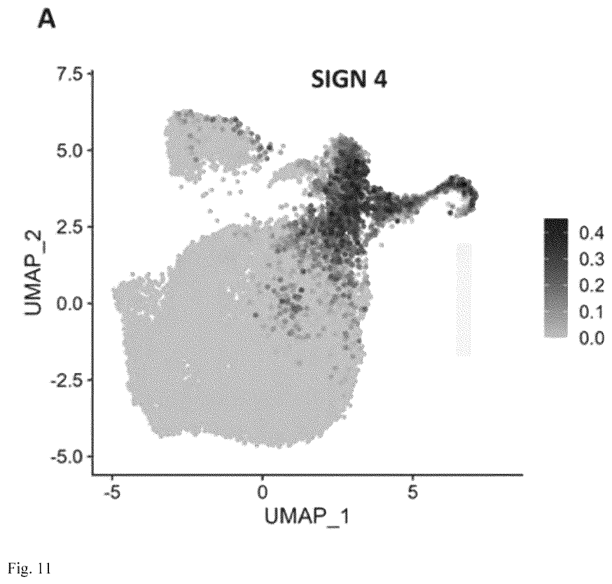

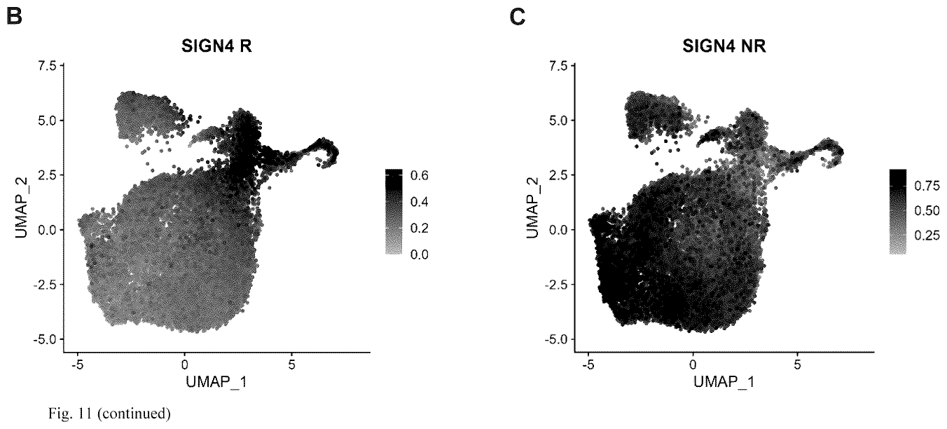

FIG. 11A shows the distribution on the UMAP from 9 pancreatic ductal adenocarcinoma (PDAC) samples of T-cells predicted to express tumor-reactive TCRs by means of the genes CXCL13 and CCL3 in combination with the genes comprised by Gene Set 2 as well as with the genes comprised by Gene Set 3 (together: Signature 4=SIGN4). The small UMAP plot in FIG. 11B shows the prediction of tumor-reactive (TR) T-cells using all 70 genes that were selected as biomarkers within SIGN4 for predicting T-cells expressing tumor-reactive (TR) TCRs. The small UMAP plot in FIG. 11C shows the prediction of tumor non-reactive (TNR) T-cells using all 20 genes that were selected within SIGN4 as biomarkers for predicting T-cells expressing tumor non-reactive (NTR) TCRs.

### 7.j Importance of all Genes Comprised by 13-Gene Signature (SIGN3) for Accurate Prediction of T-Cells Expressing Tumor-Reactive TCRs

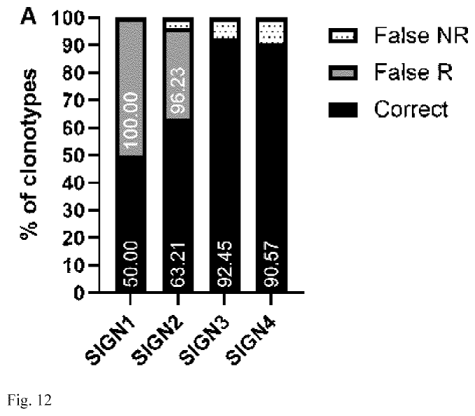

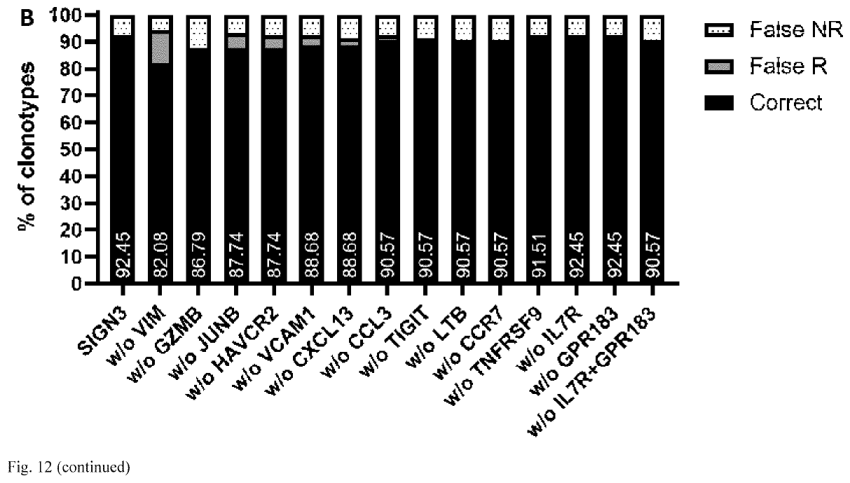

As shown in FIG. 12A, SIGN3 encompassing CXCL-13, CCL-3 and the 11 genes comprised in Gene Set 1 provides the most accurate result, as compared to the other three gene signatures, in that the correct prediction rate is 92.45%, while the false negative rate (false NR) is 7.55% and the false reactive (false R) rate 0%. FIG. 12B shows the result of similar analyses using SIGN3 in which one or two genes as indicated are omitted. As shown, each of the 13 genes comprised within SIGN3 contributes to the quality of the TCR prediction, in that the False NR and/or False R rate increases in the case one of the genes is omitted. In the case of the genes IL7R and GPR183, omission of either one of these genes does not affect the quality of the prediction. However, omission of both genes does decrease the accuracy of the prediction, indicating that these genes, while being interchangeable, both contribute to the quality of the prediction.

### 7.k Comparison of Prediction of T-Cells Expressing Reactive TCRs on Basis of Pancreatic Cancer Data Set Using 13-Gene Signature (SIGN3) and Gene Signature as Described in WO 2021/188954

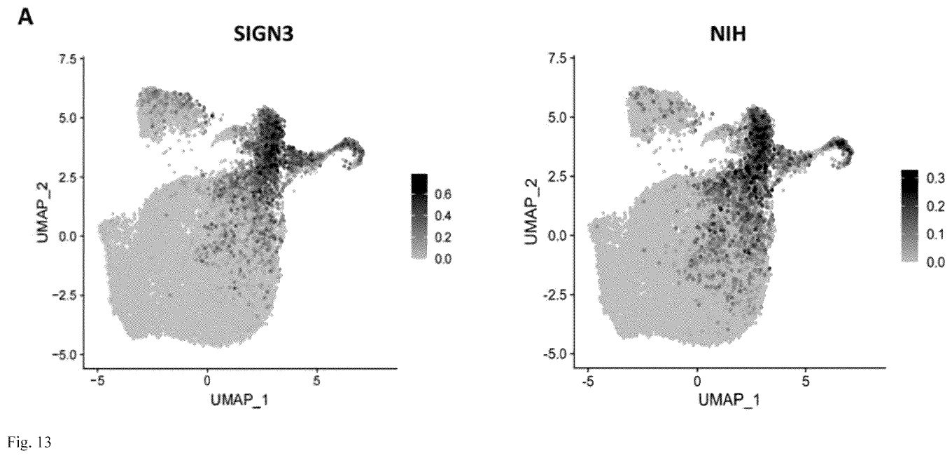

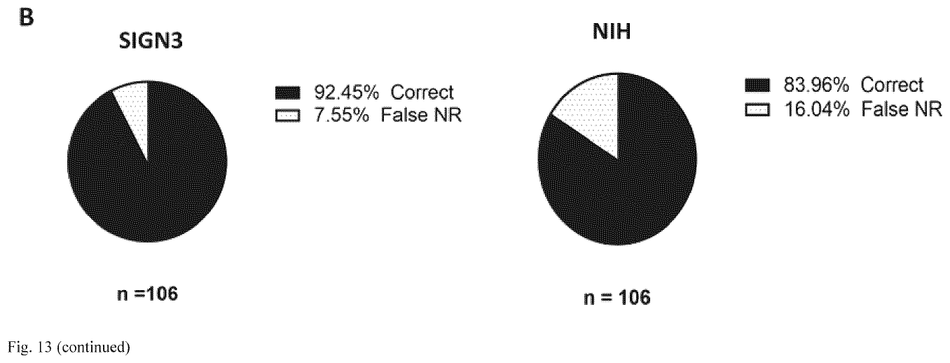

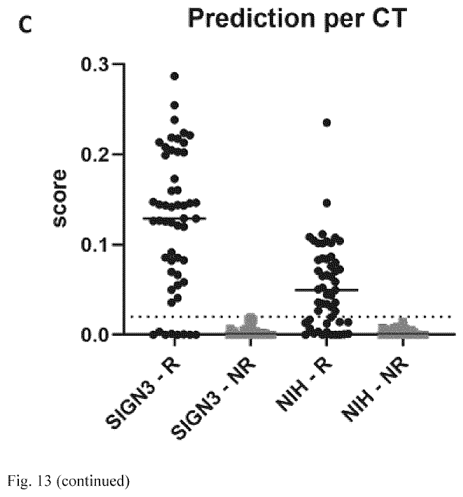

FIG. 13A shows the distribution on the UMAP from 9 pancreatic ductal adenocarcinoma (PDAC) samples of T-cells predicted to express tumor-reactive TCRs by means of either the genes CXCL13 and CCL3 in combination with the genes comprised by Gene Set 2 (together: SIGN3), or by the by the 85-gene set comprising all genes for either CD8-positive or unspecified T-cells (see legend to FIG. 13 for specific gene names) as described in NIH patent application WO 2021/188954 (NIH). As shown in FIG. 13B, the accuracy of prediction of SIGN3 is higher than that of the NIH signature. Furthermore, the discrimination between tumor-reactive (R) and non tumor-reactive (NR) T-cell clonotypes on the basis of the score for each clonotype is less ambigous when using SIGN3 (FIG. 13C).

### 7.l Comparison of Prediction of T-Cells Expressing Reactive TCRs on Basis of a Lung Cancer Data Set Using 13-Gene Signature (SIGN3) and Gene Signature as Described in WO 2021/188954

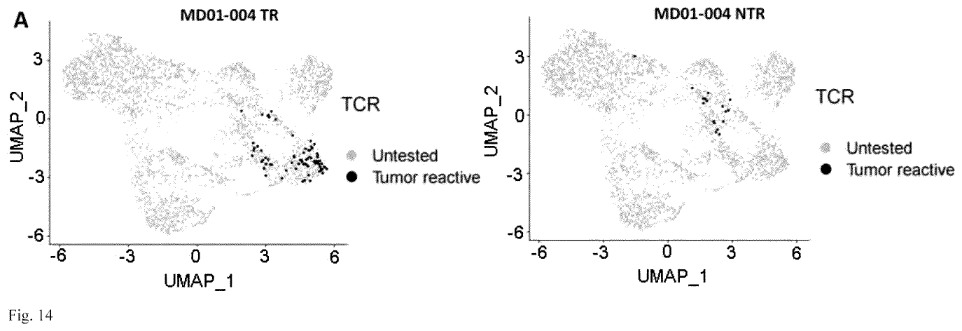

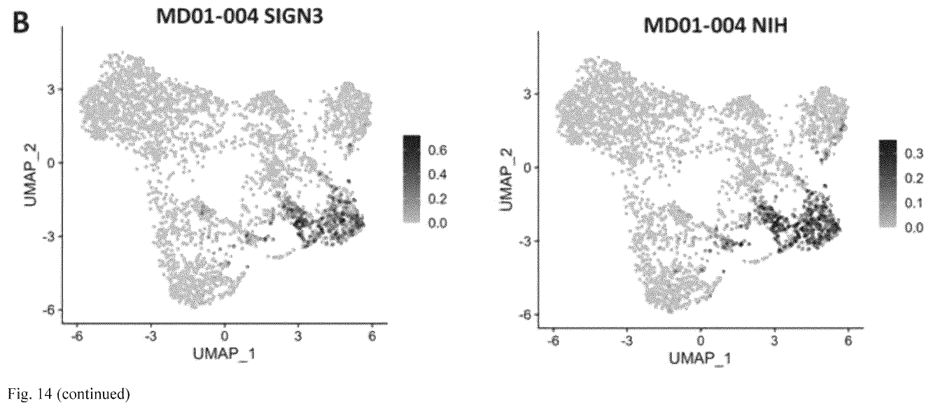

FIG. 14A, upper panel, shows the distribution of the T-cells expressing tumor-reactive (TR) T-cell receptors (TCRs) on the UMAP of T Cells from lung cancer patient MD01-004, as published by Caushi et. al. (2021), Nature 596(7870):126. The lower panel shows the distribution of the T-cells expressing tumor non-reactive (TNR) TCRs on the same UMAP. In total 19 TCRs representing unique T-cell clonotypes were tested, of which 14 were found to be tumor-reactive (TR) and 5 tumor non-reactive (TNR). FIG. 14B shows the distribution on the UMAP of lung cancer patient MDO1-004 of T-cells predicted to express tumor-reactive TCRs by means of either the genes CXCL13 and CCL3 in combination with the genes comprised by Gene Set 2 (together: SIGN3), or by the by the 85-gene set comprising all genes for either CD8-positive or unspecified T-cells (see legend to FIG. 13 for specific gene names) as described in NUH patent application WO 2021/188954 (NIH).

### REFERENCES CITED

Caushi et. al. (2021), Nature 596(7870):126
Cano-Gamez et al. (2020), Nat Comm 11: art. 1801 (doi.org/10.1038/s41467-020-15543-y)
Iwabuchi & van Kaer (2019), Front Immunol 10:1837 (doi: 10.3389/fimmu.2019.01837)
Lowery et al. (2022), Science 10.1126/science.ab15447
Magen et al. (2019), Cell Rep 29(10):3019 (doi.org/10.1016/j.celrep.2019.10.131)
MacQueen (1967), “Some methods for classification and analysis of multivariate observations”, 5th Berkeley Symposium on Mathematical Statistics and Probability
McInnes et al. (2020), arXiv:1802.03426v3
Oh et al. (2020), Cell 181(7):1612 (doi.org/10.1016/j.cell.2020.05.017)
van der Maaten and Hinton (2008), J Machine Learning Res 9:2579
WO2018/209324
WO2019/070755
WO 2021/188954

TABLE 1
Biomarkers of the core signature

| No. | Gene.Name | Signature | Full.Gene.Name | ENSEMBL ID | ENTREZ ID | Refseq.version |
|---|---|---|---|---|---|---|
| 1 | CCL4 | Core | C-C motif chemokine ligand 4 | ENSG00000275302 | 6351 | NM_002984.4 |
| 2 | CCL4L2 | Core | C-C motif chemokine ligand 4 like 2 | ENSG00000276070 | 9560 | NM_001001435.2 |
| 3 | CCL3 | Core | C-C motif chemokine ligand 3 | ENSG00000277632 | 6348 | NM_002983.3 |
| 4 | CCL3L1 | Core | C-C motif chemokine ligand 3 like 1 | ENSG00000277796 | 6349 | NM_021006.5 |
| 5 | CXCL13 | Core | C-X-C motif chemokine ligand 13 | ENSG00000156234 | 10563 | NM_001371558.1 |

TABLE 2
Biomarkers of the accessory 1 signature

| No. | Gene.Name | Signature | Full.Gene.Name | ENSEMBL ID | ENTREZ ID | Refseq.version |
|---|---|---|---|---|---|---|
| 6 | IFNG | Accessory 1 | interferon gamma(IFNG) | ENSG00000111537 | 3458 | NM_000619.3 |
| 7 | HAVCR2 | Accessory 1 | hepatitis A virus cellular receptor | ENSG00000135077 | 84868 | NM_032782.5 |
| | | | 2(HAVCR2) | | | |
| 8 | FNBP1 | Accessory 1 | formin binding protein 1(FNBP1) | ENSG00000187239 | 23048 | NM_001363755.1 |
| 9 | CSRNP1 | Accessory 1 | cysteine and serine rich nuclear protein | ENSG00000144655 | 64651 | NM_001320559.2 |
| | | | 1(CSRNP1) | | | |
| 10 | SPRY1 | Accessory 1 | sprouty RTK signaling antagonist 1(SPRY1) | ENSG00000164056 | 10252 | NM_001258038.2 |
| 11 | RHOH | Accessory 1 | ras homolog family member H(RHOH) | ENSG00000168421 | 399 | NM_001278359.2 |
| 12 | FOXN2 | Accessory 1 | forkhead box N2(FOXN2) | ENSG00000170802 | 3344 | NM_002158.4 |
| 13 | HIF1A | Accessory 1 | hypoxia inducible factor 1 alpha sub- | ENSG00000100644 | 3091 | NM_001243084.2 |
| | | | unit(HIF1A) | | | |
| 14 | TOB1 | Accessory 1 | transducer of ERBB2 1 (TOB1) | ENSG00000141232 | 10140 | NM_001243877.2 |
| 15 | RILPL2 | Accessory 1 | Rab interacting lysosomal protein like | ENSG00000150977 | 196383 | NM_145058.3 |
| | | | 2(RILPL2) | | | |
| 16 | CD8B | Accessory 1 | CD8b molecule(CD8B) | ENSG00000172116 | 926 | NM_001178100.2 |
| 17 | GABARAPL1 | Accessory 1 | GABA type A receptor associated protein | ENSG00000139112 | 23710 | NM_001363598.2 |
| | | | like 1(GABARAPL1) | | | |
| 18 | TNFSF14 | Accessory 1 | tumor necrosis factor superfamily member | ENSG00000125735 | 8740 | NM_003807.5 |
| | | | 14(TNFSF14) | | | |
| 19 | EGR1 | Accessory 1 | early growth response 1(EGR1) | ENSG00000120738 | 1958 | NM_001964.3 |
| 20 | EGR2 | Accessory 1 | early growth response 2(EGR2) | ENSG00000122877 | 1959 | NM_000399.5 |
| 21 | TAGAP | Accessory 1 | T-cell activation RhoGTPase activating pro- | ENSG00000164691 | 117289 | NM_001278733.2 |
| | | | tein(TAGAP) | | | |
| 22 | TNFSF9 | Accessory 1 | tumor necrosis factor superfamily member | ENSG00000125657 | 8744 | NM_003811.4 |
| | | | 9(TNFSF9) | | | |
| 23 | ANXA1 | Accessory 1 | annexin A1(ANXA1) | ENSG00000135046 | 301 | NM_000700.3 |
| 24 | MAP3K8 | Accessory 1 | mitogen-activated protein kinase kinase ki- | ENSG00000107968 | 1326 | NM_001244134.1 |
| | | | nase 8(MAP3K8) | | | |
| 25 | PIK3R1 | Accessory 1 | phosphoinositide-3-kinase regulatory sub- | ENSG00000145675 | 5295 | NM_001242466.2 |
| | | | unit 1(PIK3R1) | | | |
| 26 | DUSP2 | Accessory 1 | dual specificity phosphatase 2(DUSP2) | ENSG00000158050 | 1844 | NM_004418.4 |
| 27 | DUSP4 | Accessory 1 | dual specificity phosphatase 4(DUSP4) | ENSG00000120875 | 1846 | NM_001394.7 |
| 28 | DUSP6 | Accessory 1 | dual specificity phosphatase 6(DUSP6) | ENSG00000139318 | 1848 | NM_001946.4 |
| 29 | CLIC3 | Accessory 1 | chloride intracellular channel 3(CLIC3) | ENSG00000169583 | 9022 | NM_004669.3 |
| 30 | RASGEF1B | Accessory 1 | RasGEF domain family member | ENSG00000138670 | 153020 | NM_001300735.2 |
| | | | 1B(RASGEF1B) | | | |
| 31 | LAG3 | Accessory 1 | lymphocyte activating 3(LAG3) | ENSG00000089692 | 3902 | NM_002286.6 |
| 32 | XCL2 | Accessory 1 | X-C motif chemokine ligand 2(XCL2) | ENSG00000143185 | 6846 | NM_003175.4 |
| 33 | NR4A2 | Accessory 1 | nuclear receptor subfamily 4 group A mem- | ENSG00000153234 | 4929 | NM_006186.4 |
| | | | ber 2(NR4A2) | | | |
| 34 | DNAJB6 | Accessory 1 | DnaJ heat shock protein family (Hsp40) | ENSG00000105993 | 10049 | NM_001363676.1 |
| | | | member B6(DNAJB6) | | | |
| 35 | NFKBID | Accessory 1 | NFKB inhibitor delta(NFKBID) | ENSG00000167604 | 84807 | NM_001321831.2 |
| 36 | MCL1 | Accessory 1 | BCL2 family apoptosis regulator(MCL1) | ENSG00000143384 | 4170 | NM_001197320.2 |
| 37 | EVI2A | Accessory 1 | ecotropic viral integration site 2A(EVI2A) | ENSG00000126860 | 2123 | NM_001003927.3 |
| 38 | SLC7A5 | Accessory 1 | solute carrier family 7 member 5(SLC7A5) | ENSG00000103257 | 8140 | NM_003486.7 |
| 39 | H3F3B | Accessory 1 | H3 histone family member 3B(H3F3B) | ENSG00000132475 | 3021 | NM_005324.5 |
| 40 | NR4A3 | Accessory 1 | nuclear receptor subfamily 4 group A mem- | ENSG00000119508 | 8013 | NM_006981.4 |
| | | | ber 3(NR4A3) | | | |
| 41 | REL | Accessory 1 | REL proto-oncogene NF-kB subunit(REL) | ENSG00000162924 | 5966 | NM_001291746.2 |
| 42 | IRF4 | Accessory 1 | interferon regulatory factor 4(IRF4) | ENSG00000137265 | 3662 | NM_001195286.2 |
| 43 | CST7 | Accessory 1 | cystatin F(CST7) | ENSG00000077984 | 8530 | NM_003650.4 |
| 44 | ATF3 | Accessory 1 | activating transcription factor 3(ATF3) | ENSG00000162772 | 467 | NM_001030287.4 |
| 45 | TNF | Accessory 1 | tumor necrosis factor(TNF) | ENSG00000232810 | 7124 | NM_000594.4 |
| 46 | GPR171 | Accessory 1 | G protein-coupled receptor 171(GPR171) | ENSG00000174946 | 29909 | NM_013308.4 |
| 47 | BCL2A1 | Accessory 1 | BCL2 related protein A1(BCL2A1) | ENSG00000140379 | 597 | NM_001114735.2 |
| 48 | ITGA1 | Accessory 1 | integrin subunit alpha 1(ITGA1) | ENSG00000213949 | 3672 | NM_181501.2 |
| 49 | TNFAIP3 | Accessory 1 | TNF alpha induced protein 3(TNFAIP3) | ENSG00000118503 | 7128 | NM_001270507.2 |
| 50 | NR4A1 | Accessory 1 | nuclear receptor subfamily 4 group A mem- | ENSG00000123358 | 3164 | NM_001202233.2 |
| | | | ber 1(NR4A1) | | | |
| 51 | RUNX3 | Accessory 1 | runt related transcription factor 3(RUNX3) | ENSG00000020633 | 864 | NM_001031680.2 |
| 52 | HERPUD2 | Accessory 1 | HERPUD family member 2(HERPUD2) | ENSG00000122557 | 64224 | NM_022373.5 |
| 53 | FASLG | Accessory 1 | Fas ligand(FASLG) | ENSG00000117560 | 356 | NM_000639.3 |
| 54 | CBLB | Accessory 1 | Cbl proto-oncogene B(CBLB) | ENSG00000114423 | 868 | NM_001321786.1 |
| 55 | PTGER4 | Accessory 1 | prostaglandin E receptor 4(PTGER4) | ENSG00000171522 | 5734 | NM_000958.3 |
| 56 | SLA | Accessory 1 | Src-like-adaptor(SLA) | ENSG00000155926 | 6503 | NM_001045556.3 |
| 57 | XCL1 | Accessory 1 | X-C motif chemokine ligand 1(XCL1) | ENSG00000143184 | 6375 | NM_002995.3 |
| 58 | BHLHE40 | Accessory 1 | basic helix-loop-helix family member | ENSG00000134107 | 8553 | NM_003670.3 |
| | | | e40(BHLHE40) | | | |
| 59 | LYST | Accessory 1 | lysosomal trafficking regulator(LYST) | ENSG00000143669 | 1130 | NM_000081.4 |
| 60 | KLRD1 | Accessory 1 | killer cell lectin like receptor D1(KLRD1) | ENSG00000134539 | 3824 | NM_001114396.3 |
| 61 | ZNF682 | Accessory 1 | zinc finger protein 682(ZNF682) | ENSG00000197124 | 91120 | NM_001077349.1 |
| 62 | CTSW | Accessory 1 | cathepsin W(CTSW) | ENSG00000172543 | 1521 | NM_001335.4 |
| 63 | SLC2A3 | Accessory 1 | solute carrier family 2 member 3(SLC2A3) | ENSG00000059804 | 6515 | NM_006931.3 |
| 64 | NLRP3 | Accessory 1 | NLR family pyrin domain containing | ENSG00000162711 | 114548 | NM_001079821.3 |
| | | | 3(NLRP3) | | | |
| 65 | SCML4 | Accessory 1 | sex comb on midleg-like 4 (Drosoph- | ENSG00000146285 | 256380 | NM_001286408.2 |
| | | | ila)(SCML4) | | | |
| 66 | VSIR | Accessory 1 | V-Set Immunoregulator Receptor (VSIR) | ENSG00000107738 | 64115 | NM_022153.2 |
| 67 | LINC01871 | Accessory 1 | Long Intergenic Non-Protein Coding RNA | ENSG00000235576 | 101929531 | XR_001739273.1 |
| | | | 1871 (LINC01871) | | | |
| 68 | ZFP36L1 | Accessory 1 | ZFP36 Ring Finger Protein Like 1 | ENSG00000185650 | 677 | NM_001244698.2 |

TABLE 3
Biomarkers of the accessory 2 signature

| | | | | | ENTREZ | |
|---|---|---|---|---|---|---|
| No. | Gene. Name | Signature | Full.Gene.Name | ENSEMBL ID | ID | Refseq. version |
| 69 | CCL5 | Accessory 2 | C-C motif chemokine ligand 5(CCL5) | ENSG00000271503 | 6352 | NM_001278736.2 |
| 70 | GZMH | Accessory 2 | granzyme H(GZMH) | ENSG00000100450 | 2999 | NM_001270780.2 |
| 71 | CLEC2B | Accessory 2 | C-type lectin domain family 2 member | ENSG00000110852 | 9976 | NM_005127.3 |
| | | | B(CLEC2B) | | | |
| 72 | GZMA | Accessory 2 | granzyme A(GZMA) | ENSG00000145649 | 3001 | NM_006144.4 |
| 73 | CD69 | Accessory 2 | CD69 molecule(CD69) | ENSG00000110848 | 969 | NM_001781.2 |
| 74 | GZMK | Accessory 2 | granzyme K(GZMK) | ENSG00000113088 | 3003 | NM_002104.3 |
| 75 | CRTAM | Accessory 2 | cytotoxic and regulatory T-cell mole- | ENSG00000109943 | 56253 | NM_001304782.2 |
| | | | cule(CRTAM) | | | |

TABLE 4
Biomarkers of the exclusion signature

| No. | Gene.Name | Signature | Full.Gene.Name | ENSEMBL ID | ENTREZ ID | Refseq.version |
|---|---|---|---|---|---|---|
| 76 | GNLY | Exclusion | granulysin | ENSG00000115523 | 10578 | NM_001302758.2 |
| 77 | FGFBP2 | Exclusion | fibroblast growth factor | ENSG00000137441 | 83888 | NM_031950.4 |
| | | | binding protein 2 | | | |

TABLE 5
Additional biomarkers of Gene Set 2; for Prediction, R means expression is predictive
of a reactive cell, NR means expression is predictive of a non-reactive cell.

| No. | Gene.Name | Prediction | Full.Gene.Name | ENSEMBL ID | ENTREZ ID | Refseq.version |
|---|---|---|---|---|---|---|
| 78 | TNFRSF9 | R | TNF Receptor Superfamily Member 9 | ENSG00000049249 | 3604 | NM_001561.6 |
| 79 | VCAM1 | R | Vascular Cell Adhesion Molecule 1 | ENSG00000162692 | 7412 | NM_001078.4 |
| 80 | TIGIT | R | T Cell Immunoreceptor With Ig And | ENSG00000181847 | 201633 | NM_173799.4 |
| | | | ITIM Domains | | | |
| 81 | HAVCR2 | R | hepatitis A virus cellular receptor | ENSG00000135077 | 84868 | NM_032782.5 |
| | | | 2(HAVCR2) | | | |
| 82 | GZMB | R | Granzyme B | ENSG00000100453 | 3002 | NM_004131.6 |
| 83 | GPR183 | NR | G Protein-Coupled Receptor 183 | ENSG00000169508 | 1880 | NM_004951.5 |
| 84 | CCR7 | NR | C-C Motif Chemokine Receptor 7 | ENSG00000126353 | 1236 | NM_001838.4 |
| 85 | IL7R | NR | Interleukin 7 Receptor | ENSG00000168685 | 3575 | NM_002185.5 |
| 86 | VIM | NR | Vimentin | ENSG00000026025 | 7431 | NM_003380.5 |
| 87 | LTB | NR | Lymphotoxin Beta | ENSG00000227507 | 4050 | NM_002341.2 |
| 88 | JUNB | NR | JunB Proto-Oncogene, AP-1 Transcrip- | ENSG00000171223 | 3726 | NM_002229.3 |
| | | | tion Factor Subunit | | | |

TABLE 6
Biomarkers of Gene Set 3; for Prediction, R means expression is predictive of
a reactive cell, NR means expression is predictive of a non-reactive cell.

| No. | Gene.Name | Prediction | Full.Gene.Name | ENSEMBL ID | ENTREZ ID | Refseq.version |
|---|---|---|---|---|---|---|
| 89 | ACP5 | R | Acid Phosphatase 5, Tartrate Resistant | ENSG00000102575 | 54 | NM_001611.5 |
| 90 | NKG7 | R | Natural Killer Cell Granule Protein 7 | ENSG00000105374 | 4818 | NM_005601.4 |
| 91 | KRT86 | R | Keratin 86 | ENSG00000170442 | 3829 | NM_001320198.2 |
| 92 | LAYN | R | Layilin | ENSG00000204381 | 143903 | NM_178834.5 |
| 93 | HLA-DRB5 | R | Major Histocompatibility Complex, | ENSG00000198502 | 3127 | NM_002125.4 |
| | | | Class II, DR Beta 5 | | | |
| 94 | CTLA4 | R | Cytotoxic T-Lymphocyte Associated | ENSG00000163599 | 1493 | NM_005214.5 |
| | | | Protein 4 | | | |
| 95 | HLA-DRB1 | R | Major Histocompatibility Complex, | ENSG00000196126 | 3123 | NM_002124.4 |
| | | | Class II, DR Beta 1 | | | |
| 96 | IGFLR1 | R | IGF like family receptor 1 | ENSG00000126246 | 79713 | NM_024660.4 |
| 97 | HLA-DRA | R | Major Histocompatibility Complex, | ENSG00000204287 | 3122 | NM_019111.5 |
| | | | Class II, DR Alpha | | | |
| 98 | LAG3 | R | Lymphocyte Activating 3 | ENSG00000089692 | 3902 | NM_002286.6 |
| 99 | GEM | R | GTP Binding Protein Overexpressed In | ENSG00000164949 | 2669 | NM_005261.4 |
| | | | Skeletal Muscle | | | |
| 100 | LYST | R | Lysosomal Trafficking Regulator | ENSG00000143669 | 1130 | NM_000081.4 |
| 101 | GAPDH | R | Glyceraldehyde-3-Phosphate Dehydro- | ENSG00000111640 | 2597 | NM_002046.7 |
| | | | genase | | | |
| 102 | CD74 | R | CD47 Molecule | ENSG00000196776 | 961 | NM_001777.4 |
| 103 | HMOX1 | R | Heme Oxygenase 1 | ENSG00000100292 | 3162 | NM_002133.3 |
| 104 | HLA-DPA1 | R | Major Histocompatibility Complex, | ENSG00000231389 | 3113 | NM_033554.3 |
| | | | Class II, DP Alpha 1 | | | |
| 105 | DUSP4 | R | Dual Specificity Phosphatase 4 | ENSG00000120875 | 1846 | NM_001394.7 |
| 106 | CD27 | R | CD27 Molecule | ENSG00000139193 | 939 | NM_001242.5 |
| 107 | ENTPD1 | R | Ectonucleoside Triphosphate Diphospho- | ENSG00000138185 | 953 | NM_001776.6 |
| | | | hydrolase 1 | | | |
| 108 | AC243829.4 | R | CCL3 Antisense RNA 1 (IncRNA) | ENSG00000277089.4 | 102724850 | XR_001756610.1 |
| 109 | HLA-DPB1 | R | Major Histocompatibility Complex, | ENSG00000223865 | 3115 | NM_002121.6 |
| | | | Class II, DP Beta 1 | | | |
| 110 | GZMH | R | Granzyme H | ENSG00000100450 | 2999 | NM_033423.5 |
| 111 | KIR2DL4 | R | Killer Cell Immunoglobulin Like Re- | ENSG00000189013 | 3805 | NM_002255.6 |
| | | | ceptor, Two Ig Domains And Long Cy- | | | |
| | | | toplasmic Tail 4 | | | |
| 112 | CARD16 | R | Caspase Recruitment Domain Family | ENSG00000204397 | 114769 | NM_052889.4 |
| | | | Member 16 | | | |
| 113 | HLA-DQA1 | R | Major Histocompatibility Complex, | ENSG00000196735 | 3117 | NM_002122.5 |
| | | | Class II, DQ Alpha 1 | | | |
| 114 | CCL5 | R | C-C Motif Chemokine Ligand 5 | ENSG00000271503 | 6352 | NM_001278736.2 |
| 115 | CST7 | R | Cystatin F | ENSG00000077984 | 8530 | NM_003650.4 |
| 116 | LINC01943 | R | Long Intergenic Non-Protein Coding | ENSG00000280721 | 101928173 | XR_245039.3 |
| | | | RNA 1943 | | | |
| 117 | PLPP1 | R | Phospholipid Phosphatase 1 | ENSG00000067113 | 8611 | NM_003711.4 |
| 118 | CTSC | R | Cathepsin C | ENSG00000109861 | 1075 | NM_001814.6 |
| 119 | PRF1 | R | Perforin 1 | ENSG00000180644 | 5551 | NM_001083116.3 |
| 120 | MTSS1 | R | MTSS I-BAR Domain Containing 1 | ENSG00000170873 | 9788 | NM_014751.6 |
| 121 | FKBP1A | R | FKBP Prolyl Isomerase 1A | ENSG00000088832 | 2280 | NM_000801.5 |
| 122 | CXCR6 | R | C-X-C Motif Chemokine Receptor 6 | ENSG00000172215 | 10663 | NM_006564.2 |
| 123 | HLA-DMA | R | Major Histocompatibility Complex, | ENSG00000204257 | 3108 | NM_006120.4 |
| | | | Class II, DM Alpha | | | |
| 124 | ATP8B4 | R | ATPase Phospholipid Transporting 8B4 | ENSG00000104043 | 79895 | NM_024837.4 |
| 125 | GZMA | R | Granzyme A | ENSG00000145649 | 3001 | NM_006144.4 |
| 126 | GALNT2 | R | Polypeptide N-Acetylgalactosaminyl- | ENSG00000143641 | 2590 | NM_004481.5 |
| | | | transferase 2 | | | |
| 127 | CHST12 | R | Carbohydrate Sulfotransferase 12 | ENSG00000136213 | 55501 | NM_018641.5 |
| 128 | SNAP47 | R | Synaptosome Associated Protein 47 | ENSG00000143740 | 116841 | NM_053052.4 |
| 129 | TNFRSF18 | R | TNF Receptor Superfamily Member 18 | ENSG00000186891 | 8784 | NM_004195.3 |
| 130 | SIRPG | R | Signal Regulatory Protein Gamma | ENSG00000089012 | 55423 | NM_018556.4 |
| 131 | CD38 | R | CD38 Molecule | ENSG00000004468 | 952 | NM_001775.4 |
| 132 | RBPJ | R | Recombination Signal Binding Protein | ENSG00000168214 | 3516 | NM_015874.6 |
| | | | For Immunoglobulin Kappa J | | | |
| 133 | TNIP3 | R | TNFAIP3 Interacting Protein 3 | ENSG00000050730 | 79931 | NM_024873.6 |
| 134 | AHI1 | R | Abelson Helper Integration Site 1 | ENSG00000135541 | 54806 | NM_001134831.2 |
| 135 | NDFIP2 | R | Nedd4 Family Interacting Protein 2 | ENSG00000102471 | 54602 | NM_019080.3 |
| 136 | FABP5 | R | Fatty Acid Binding Protein 5 | ENSG00000164687 | 2171 | NM_001444.3 |
| 137 | RAB27A | R | RAB27A, Member RAS Oncogene | ENSG00000069974 | 5873 | NM_183235.3 |
| | | | Family | | | |
| 138 | ADGRG1 | R | Adhesion G Protein-Coupled Receptor | ENSG00000205336 | 9289 | NM_201525.4 |
| | | | G1 | | | |
| 139 | CTSW | R | Cathepsin W | ENSG00000172543 | 1521 | NM_001335.4 |
| 140 | APOBEC3G | R | Apolipoprotein B MRNA Editing En- | ENSG00000239713 | 60489 | NM_021822.4 |
| | | | zyme Catalytic Subunit 3G | | | |
| 141 | IFNG | R | Interferon Gamma | ENSG00000111537 | 3458 | NM_000619.3 |
| 142 | CTSD | R | Cathepsin D | ENSG00000117984 | 1509 | NM_001909.5 |
| 143 | PKM | R | Pyruvate Kinase M1/2 | ENSG00000067225 | 5315 | NM_002654.6 |
| 144 | NAB1 | R | NGFI-A Binding Protein 1 | ENSG00000138386 | 4664 | NM_005966.4 |
| 145 | PSMB9 | R | Proteasome 20S Subunit Beta 9 | ENSG00000240065 | 5698 | NM_002800.5 |
| 146 | PARK7 | R | Parkinsonism Associated Deglycase | ENSG00000116288 | 11315 | NM_007262.5 |
| 147 | KLRD1 | R | Killer Cell Lectin Like Receptor D1 | ENSG00000134539 | 3824 | NM_002262.5 |
| 148 | ASXL2 | R | ASXL Transcriptional Regulator 2 | ENSG00000143970 | 55252 | NM_018263.6 |
| 149 | KLRC2 | R | Killer Cell Lectin Like Receptor C2 | ENSG00000205809 | 3822 | NM_002260.4 |
| 150 | LAIR2 | R | Leukocyte Associated Immunoglobulin | ENSG00000167618 | 3904 | NM_002288.6 |
| | | | Like Receptor 2 | | | |
| 151 | FAM3C | R | FAM3 Metabolism Regulating Signal- | ENSG00000196937 | 10447 | NM_014888.3 |
| | | | ing Molecule C | | | |
| 152 | ZFP36 | NR | ZFP36 Ring Finger Protein | ENSG00000128016 | 7538 | NM_003407.5 |
| 153 | FTH1 | NR | Ferritin Heavy Chain 1 | ENSG00000167996 | 2495 | NM_002032.3 |
| 154 | FOS | NR | Fos Proto-Oncogene, AP-1 Transcrip- | ENSG00000170345 | 2353 | NM_005252.4 |
| | | | tion Factor Subunit | | | |
| 155 | ZFP36L2 | NR | ZFP36 Ring Finger Protein Like 2 | ENSG00000152518 | 678 | NM_006887.5 |
| 156 | ANXA1 | NR | Annexin A1 | ENSG00000135046 | 301 | NM_000700.3 |
| 157 | CD55 | NR | CD55 Molecule (Cromer Blood Group) | ENSG00000196352 | 1604 | NM_000574.5 |
| 158 | SLC2A3 | NR | Solute Carrier Family 2 Member 3 | ENSG00000059804 | 6515 | NM_006931.3 |
| 159 | LMNA | NR | Lamin A/C | ENSG00000160789 | 4000 | NM_170707.4 |
| 160 | CRYBG1 | NR | Crystallin Beta-Gamma Domain Con- | ENSG00000112297 | 202 | NM_001371242.2 |
| | | | taining 1 | | | |
| 161 | DUSP1 | NR | Dual Specificity Phosphatase 1 | ENSG00000120129 | 1843 | NM_004417.4 |
| 162 | PTGER4 | NR | Prostaglandin E Receptor 4 | ENSG00000171522 | 5734 | NM_000958.3 |
| 163 | MYADM | NR | Myeloid Associated Differentiation | ENSG00000179820 | 91663 | NM_138373.5 |
| | | | Marker | | | |
| 164 | BTG2 | NR | BTG Anti-Proliferation Factor 2 | ENSG00000159388 | 7832 | NM_006763.3 |
| 165 | NFKBIA | NR | NFKB Inhibitor Alpha | ENSG00000100906 | 4792 | NM_020529.3 |

TABLE 7
Genes/biomarkers associated with T-cells expressing tumor-reactive TCRs

| | | | | ENTREZ | |
|---|---|---|---|---|---|
| Rank | Gene symbol | Gene Name | ENSEMBL ID | Gene ID | AUC |
| 1 | TNFRSF9 | TNF receptor superfamily member 9(TNFRSF9) | ENSG00000049249 | 3604 | 0.758 |
| 2 | VCAM1 | vascular cell adhesion molecule 1(VCAM1) | ENSG00000162692 | 7412 | 0.741 |
| 3 | TIGIT | T cell immunoreceptor with Ig and ITIM domains(TIGIT) | ENSG00000181847 | 201633 | 0.821 |
| 4 | HAVCR2 | hepatitis A virus cellular receptor 2(HAVCR2) | ENSG00000135077 | 84868 | 0.766 |
| 5 | GZMB | granzyme B(GZMB) | ENSG00000100453 | 3002 | 0.841 |
| 6 | ACP5 | acid phosphatase 5, tartrate resistant(ACP5) | ENSG00000102575 | 54 | 0.76 |
| 7 | NKG7 | natural killer cell granule protein 7(NKG7) | ENSG00000105374 | 4818 | 0.886 |
| 8 | KRT86 | keratin 86(KRT86) | ENSG00000170442 | 3892 | 0.71 |
| 9 | LAYN | layilin(LAYN) | ENSG00000204381 | 143903 | 0.712 |
| 10 | HLA-DRB5 | major histocompatibility complex, class II, DR beta 5(HLA- | ENSG00000198502 | 3127 | 0.775 |
| | | DRB5) | | | |
| 11 | CTLA4 | cytotoxic T-lymphocyte associated protein 4(CTLA4) | ENSG00000163599 | 1493 | 0.761 |
| 12 | HLA-DRB1 | major histocompatibility complex, class II, DR beta 1(HLA- | ENSG00000206240 | 3123 | 0.79 |
| | | DRB1) | | | |
| 13 | IGFLR1 | IGF like family receptor 1(IGFLR1) | ENSG00000126246 | 79713 | 0.755 |
| 14 | HLA-DRA | major histocompatibility complex, class II, DR alpha(HLA-DRA) | ENSG00000234794 | 3122 | 0.76 |
| 15 | LAG3 | lymphocyte activating 3(LAG3) | ENSG00000089692 | 3902 | 0.771 |
| 16 | GEM | GTP binding protein overexpressed in skeletal muscle(GEM) | ENSG00000164949 | 2669 | 0.718 |
| 17 | CXCL13 | C-X-C motif chemokine ligand 13(CXCL13) | ENSG00000156234 | 10563 | 0.721 |
| 18 | LYST | lysosomal trafficking regulator(LYST) | ENSG00000143669 | 1130 | 0.757 |
| 19 | GAPDH | glyceraldehyde-3-phosphate dehydrogenase(GAPDH) | ENSG00000111640 | 2597 | 0.792 |
| 20 | CD74 | CD74 molecule(CD74) | ENSG00000019582 | 972 | 0.782 |
| 21 | HMOX1 | heme oxygenase 1(HMOX1) | ENSG00000100292 | 3162 | 0.638 |
| 22 | HLA-DPA1 | major histocompatibility complex, class II, DP alpha 1(HLA- | ENSG00000236177 | 3113 | 0.765 |
| | | DPA1) | | | |
| 23 | DUSP4 | dual specificity phosphatase 4(DUSP4) | ENSG00000120875 | 1846 | 0.775 |
| 24 | CD27 | CD27 molecule(CD27) | ENSG00000139193 | 939 | 0.738 |
| 25 | ENTPD1 | ectonucleoside triphosphate diphosphohydrolase 1(ENTPD1) | ENSG00000138185 | 953 | 0.685 |
| 26 | AC243829.4 | CCL3 Antisense RNA 1(CCL3-AS1) | ENSG00000277089 | 102724850 | 0.652 |
| 27 | HLA-DPB1 | major histocompatibility complex, class II, DP beta 1(HLA-DPB1) | ENSG00000236693 | 3115 | 0.755 |
| 28 | GZMH | granzyme H(GZMH) | ENSG00000100450 | 2999 | 0.751 |
| 29 | KIR2DL4 | killer cell immunoglobulin like receptor, two Ig domains and long | ENSG00000277540 | 3805 | 0.628 |
| | | cytoplasmic tail 4(KIR2DL4) | | | |
| 30 | CARD16 | caspase recruitment domain family member 16(CARD16) | ENSG00000204397 | 114769 | 0.73 |
| 31 | HLA-DQA1 | major histocompatibility complex, class II, DQ alpha 1(HLA- | ENSG00000196735 | 3117 | 0.701 |
| | | DQA1) | | | |
| 32 | CCL5 | C-C motif chemokine ligand 5(CCL5) | ENSG00000271503 | 6352 | 0.764 |
| 33 | CST7 | cystatin F(CST7) | ENSG00000077984 | 8530 | 0.747 |
| 34 | LINC01943 | long intergenic non-protein coding RNA 1943(LINC01943) | ENSG00000280721 | 101928173 | 0.652 |
| 35 | PLPP1 | phospholipid phosphatase 1(PLPP1) | ENSG00000067113 | 8611 | 0.658 |
| 36 | CTSC | cathepsin C(CTSC) | ENSG00000109861 | 1075 | 0.727 |
| 37 | PRF1 | perforin 1(PRF1) | ENSG00000180644 | 5551 | 0.738 |
| 38 | MTSS1 | MTSS I-BAR domain containing 1(MTSS1) | ENSG00000170873 | 9788 | 0.636 |
| 39 | FKBP1A | FKBP prolyl isomerase 1A(FKBP1A) | ENSG00000088832 | 2280 | 0.724 |
| 40 | CXCR6 | C-X-C motif chemokine receptor 6(CXCR6) | ENSG00000172215 | 10663 | 0.722 |
| 41 | HLA-DMA | major histocompatibility complex, class II, DM alpha(HLA-DMA) | ENSG00000241394 | 3108 | 0.669 |
| 42 | ATP8B4 | ATPase phospholipid transporting 8B4 (putative)(ATP8B4) | ENSG00000104043 | 79895 | 0.62 |
| 43 | GZMA | granzyme A(GZMA) | ENSG00000145649 | 3001 | 0.734 |
| 44 | GALNT2 | polypeptide N-acetylgalactosaminyltransferase 2(GALNT2) | ENSG00000143641 | 2590 | 0.686 |
| 45 | CHST12 | carbohydrate sulfotransferase 12(CHST12) | ENSG00000136213 | 55501 | 0.691 |
| 46 | SNAP47 | synaptosome associated protein 47(SNAP47) | ENSG00000143740 | 116841 | 0.655 |
| 47 | INFRSF18 | TNF receptor superfamily member 18(TNFRSF18) | ENSG00000186891 | 8784 | 0.655 |
| 48 | SIRPG | signal regulatory protein gamma(SIRPG) | ENSG00000089012 | 55423 | 0.675 |
| 49 | CD38 | CD38 molecule(CD38) | ENSG00000004468 | 952 | 0.645 |
| 50 | RBPJ | recombination signal binding protein for immunoglobulin kappa J | ENSG00000168214 | 3516 | 0.71 |
| | | region(RBPJ) | | | |
| 51 | TNIP3 | TNFAIP3 interacting protein 3(TNIP3) | ENSG00000050730 | 79931 | 0.675 |
| 52 | AHI1 | Abelson helper integration site 1(AHI1) | ENSG00000135541 | 54806 | 0.679 |
| 53 | NDFIP2 | Nedd4 family interacting protein 2(NDFIP2) | ENSG00000102471 | 54602 | 0.659 |
| 54 | FABP5 | fatty acid binding protein 5(FABP5) | ENSG00000164687 | 2171 | 0.69 |
| 55 | RAB27A | RAB27A, member RAS oncogene family(RAB27A) | ENSG00000069974 | 5873 | 0.697 |
| 56 | ADGRG1 | adhesion G protein-coupled receptor G1(ADGRG1) | ENSG00000205336 | 9289 | 0.62 |
| 57 | CTSW | cathepsin W(CTSW) | ENSG00000172543 | 1521 | 0.714 |
| 58 | APOBEC3G | apolipoprotein B mRNA editing enzyme catalytic subunit | ENSG00000239713 | 60489 | 0.689 |
| | | 3G(APOBEC3G) | | | |
| 59 | CCL3 | C-C motif chemokine ligand 3(CCL3) | ENSG00000278567 | 6348 | 0.645 |
| 60 | IFNG | interferon gamma(IFNG) | ENSG00000111537 | 3458 | 0.674 |
| 61 | CTSD | cathepsin D(CTSD) | ENSG00000117984 | 1509 | 0.702 |
| 62 | PKM | pyruvate kinase M1/2(PKM) | ENSG00000067225 | 5315 | 0.687 |
| 63 | NAB1 | NGFI-A binding protein 1(NAB1) | ENSG00000138386 | 4664 | 0.631 |
| 64 | PSMB9 | proteasome 20S subunit beta 9(PSMB9) | ENSG00000243594 | 5698 | 0.699 |
| 65 | PARK7 | Parkinsonism associated deglycase(PARK7) | ENSG00000116288 | 11315 | 0.694 |
| 66 | KLRD1 | killer cell lectin like receptor D1(KLRD1) | ENSG00000134539 | 3824 | 0.682 |
| 67 | ASXL2 | ASXL transcriptional regulator 2(ASXL2) | ENSG00000143970 | 55252 | 0.662 |
| 68 | KLRC2 | killer cell lectin like receptor C2(KLRC2) | ENSG00000205809 | 3822 | 0.636 |
| 69 | LAIR2 | leukocyte associated immunoglobulin like receptor 2(LAIR2) | ENSG00000277335 | 3904 | 0.584 |
| 70 | FAM3C | FAM3 metabolism regulating signaling molecule C(FAM3C) | ENSG00000196937 | 10447 | 0.635 |
| 71 | PGAM1 | phosphoglycerate mutase 1(PGAM1) | ENSG00000171314 | 5223 | 0.683 |
| 72 | HLA-DQB1 | major histocompatibility complex, class II, DQ beta 1(HLA- | ENSG00000231286 | 3119 | 0.651 |
| | | DQB1) | | | |
| 73 | BATF | basic leucine zipper ATF-like transcription factor(BATF) | ENSG00000156127 | 10538 | 0.669 |
| 74 | CD63 | CD63 molecule(CD63) | ENSG00000135404 | 967 | 0.68 |
| 75 | LINC02195 | long intergenic non-protein coding RNA 2195(LINC02195) | ENSG00000236481 | 105371152 | 0.623 |
| 76 | CYTOR | cytoskeleton regulator RNA(CYTOR) | ENSG00000222041 | 112597 | 0.679 |
| 77 | SLA2 | Src like adaptor 2(SLA2) | ENSG00000101082 | 84174 | 0.666 |
| 78 | ID3 | inhibitor of DNA binding 3, HLH protein(ID3) | ENSG00000117318 | 3399 | 0.617 |
| 79 | LINC01871 | long intergenic non-protein coding RNA 1871(LINC01871) | ENSG00000235576 | 101929531 | 0.664 |
| 80 | CLEC2B | C-type lectin domain family 2 member B(CLEC2B) | ENSG00000110852 | 9976 | 0.665 |

TABLE 8
Genes/biomarkers associated with T-cells expressing tumor non-reactive TCRs

| | Gene | | | ENTREZ | |
|---|---|---|---|---|---|
| Rank | symbol | Gene Name | ENSEMBL ID | Gene ID | AUC |
| 1 | IL 7R | interleukin 7 receptor(IL7R) | ENSG00000168685 | 3575 | 0.91 |
| 2 | ZFP36 | ZFP36 ring finger protein(ZFP36) | ENSG00000128016 | 7538 | 0.803 |
| 3 | FTH1 | ferritin heavy chain 1(FTH1) | ENSG00000167996 | 2495 | 0.79 |
| 4 | FOS | Fos proto-oncogene, AP-1 transcription factor subunit(FOS) | ENSG00000170345 | 2353 | 0.778 |
| 5 | ZFP36L2 | ZFP36 ring finger protein like 2(ZFP36L2) | ENSG00000152518 | 678 | 0.77 |
| 6 | ANXA1 | annexin A1(ANXA1) | ENSG00000135046 | 301 | 0.769 |
| 7 | GPR183 | G protein-coupled receptor 183(GPR183) | ENSG00000169508 | 1880 | 0.733 |
| 8 | CD55 | CD55 molecule (Cromer blood group)(CD55) | ENSG00000196352 | 1604 | 0.722 |
| 9 | CCR7 | C-C motif chemokine receptor 7(CCR7) | ENSG00000126353 | 1236 | 0.677 |
| 10 | VIM | vimentin(VIM) | ENSG00000026025 | 7431 | 0.726 |
| 11 | JUNB | JunB proto-oncogene, AP-1 transcription factor subunit(JUNB) | ENSG00000171223 | 3726 | 0.731 |
| 12 | SLC2A3 | solute carrier family 2 member 3(SLC2A3) | ENSG00000059804 | 6515 | 0.722 |
| 13 | LMNA | lamin A/C(LMNA) | ENSG00000160789 | 4000 | 0.692 |
| 14 | CRYBG1 | crystallin beta-gamma domain containing 1(CRYBG1) | ENSG00000112297 | 202 | 0.684 |
| 15 | LTB | lymphotoxin beta(LTB) | ENSG00000236925 | 4050 | 0.678 |
| 16 | DUSP1 | dual specificity phosphatase 1(DUSP1) | ENSG00000120129 | 1843 | 0.702 |
| 17 | PTGER4 | prostaglandin E receptor 4(PTGER4) | ENSG00000171522 | 5734 | 0.679 |
| 18 | MYADM | myeloid associated differentiation marker(MYADM) | ENSG00000179820 | 91663 | 0.691 |
| 19 | BTG2 | BTG anti-proliferation factor 2(BTG2) | ENSG00000159388 | 7832 | 0.69 |
| 20 | NFKBIA | NFKB inhibitor alpha(NFKBIA) | ENSG00000100906 | 4792 | 0.679 |
| 21 | KLF3 | Kruppel like factor 3(KLF3) | ENSG00000109787 | 51274 | 0.62 |
| 22 | FOSB | FosB proto-oncogene, AP-1 transcription factor | ENSG00000125740 | 2354 | 0.661 |
| | | subunit(FOSB) | | | |
| 23 | CD69 | CD69 molecule(CD69) | ENSG00000110848 | 969 | 0.674 |
| 24 | CSRNP1 | cysteine and serine rich nuclear protein 1(CSRNP1) | ENSG00000144655 | 64651 | 0.649 |
| 25 | CDKNIA | cyclin dependent kinase inhibitor 1A(CDKNIA) | ENSG00000124762 | 1026 | 0.654 |
| 26 | SELL | selectin L(SELL) | ENSG00000188404 | 6402 | 0.6 |
| 27 | KLF2 | Kruppel like factor 2(KLF2) | ENSG00000127528 | 10365 | 0.61 |
| 28 | SC5D | sterol-C5-desaturase(SC5D) | ENSG00000109929 | 6309 | 0.625 |
| 29 | CCNH | cyclin H(CCNH) | ENSG00000134480 | 902 | 0.654 |
| 30 | DDIT4 | DNA damage inducible transcript 4(DDIT4) | ENSG00000168209 | 54541 | 0.663 |
| 31 | PIK3R1 | phosphoinositide-3-kinase regulatory subunit 1(PIK3R1) | ENSG00000145675 | 5295 | 0.654 |
| 32 | KLF6 | Kruppel like factor 6(KLF6) | ENSG00000067082 | 1316 | 0.668 |
| 33 | SOCS1 | suppressor of cytokine signaling 1(SOCS1) | ENSG00000185338 | 8651 | 0.618 |
| 34 | TXNIP | thioredoxin interacting protein(TXNIP) | ENSG00000265972 | 10628 | 0.618 |
| 35 | ATF3 | activating transcription factor 3(ATF3) | ENSG00000162772 | 467 | 0.562 |

1. A method of identifying a T-cell reactive to cells of a subject presenting a T-cell activating antigen (reactive T-cell), comprising
(a) determining expression of at least one of CXCL13, CCL3, CCL3L1, CCL4, and CCL4L2 in T-cells from a sample of said subject; and
(b) identifying a reactive T-cell based on the determination of step (a).

2. The method of claim 1, wherein step (a) comprises determining expression of at least two of CXCL13, CCL3, CCL3L1, CCL4, and CCL4L2.

3. The method of claim 1, wherein step (a) comprises further determining expression of at least one biomarker selected from the list consisting of TNFRSF9, VCAM1, TIGIT, HAVCR2, GZMB, GPR183, CCR7, IL7R, VIM, LTB, and JUNB.

4. The method of claim 1, wherein step (a) comprises further determining expression of at least one biomarker selected from the list consisting of ACP5, NKG7, KRT86, LAYN, HLA-DRB5, CTLA4, HLA-DRB1, IGFLR1, HLA-DRA, LAG3, GEM, LYST, GAPDH, CD74, HMOX1, HLA-DPA1, DUSP4, CD27, ENTPD1, AC243829.4, HLA-DPB1, GZMH, KIR2DL4, CARD16, HLA-DQA1, CCL5, CST7, LINC01943, PLPP1, CTSC, PRF1, MTSS1, FKBP1A, CXCR6, HLA-DMA, ATP8B4, GZMA, GALNT2, CHST12, SNAP47, TNFRSF18, SIRPG, CD38, RBPJ, TNIP3, AHI1, NDFIP2, FABP5, RAB27A, ADGRG1, CTSW, APOBEC3G, IFNG, CTSD, PKM, NAB1, PSMB9, PARK7, KLRD1, ASXL2, KLRC2, LAIR2, FAM3C, ZFP36, FTH1, FOS, ZFP36L2, ANXA1, CD55, SLC2Δ3, LMNA, CRYBG1, DUSP1, PTGER4, MYADM, BTG2, and NFKBIA.

5. The method of claim 1, wherein step (a) comprises determining expression of
(i) CXCL13, CCL3 and all biomarkers listed in claim 3;
(ii) CXCL13, CCL3 and all biomarkers listed in claim 3 except IL7R; or
(iii) CXCL13, CCL3 and all biomarkers listed in claim 3 except GPR183.

6. The method of claim 1, wherein step (a) comprises determining expression of
(iv) CXCL13, CCL3, TNFRSF9, VCAM1, TIGIT, HAVCR2, GZMB, GPR183, CCR7, IL7R, VIM, LTB, JUNB, ACP5, NKG7, KRT86, LAYN, HLA-DRB5, CTLA4, HLA-DRB1, IGFLR1, HLA-DRA, LAG3, GEM, LYST, GAPDH, CD74, HMOX1, HLA-DPA1, DUSP4, CD27, ENTPD1, AC243829.4, HLA-DPB1, GZMH, KIR2DL4, CARD16, HLA-DQA1, CCL5, CST7, LINC01943, PLPP1, CTSC, PRF1, MTSS1, FKBP1A, CXCR6, HLA-DMA, ATP8B4, GZMA, GALNT2, CHST12, SNAP47, TNFRSF18, SIRPG, CD38, RBPJ, TNIP3, AHI1, NDFIP2, FABP5, RAB27A, ADGRG1, CTSW, APOBEC3G, IFNG, CTSD, PKM, NAB1, PSMB9, PARK7, KLRD1, ASXL2, KLRC2, LAIR2, FAM3C, ZFP36, FTH1, FOS, ZFP36L2, ANXA1, CD55, SLC2Δ3, LMNA, CRYBG1, DUSP1, PTGER4, MYADM, BTG2, and NFKBIA;
(v) CXCL13, CCL3 and all biomarkers listed in (iv) except IL7R; or
(vi) CXCL13, CCL3 and all biomarkers listed in (iv) except GPR183.

7. The method of claim 1, wherein said T-cell activating antigen is a cancer antigen, and wherein said sample is a tumor sample.

8. The method of claim 7, wherein said cancer is pancreatic cancer, colorectal cancer, or any other primary or metastatic solid tumor type.

9. A method of identifying a TCR binding to a T-cell activating antigen presented on a cell of a subject, said method comprising
(A) identifying a reactive T-cell according to the method of claim 1,
(B) providing the amino acid sequences of at least the complementarity determining regions (CDRs) of the TCR of the reactive T-cell identified in step (A); and, hereby,
(C) identifying a TCR binding to an activating antigen presented on a cell.

10. The method of claim 1, wherein expression of at least one biomarker of step a) is determined by single-cell sequencing.

11. The method of claim 9, wherein said method comprises further step B1) expressing a TCR comprising at least the CDRs determined in step B) in a host cell.

12. The method of claim 11, wherein said method further comprises further step B2) determining binding of the TCR expressed in step B1) to a T-cell activating antigen.

13. The method of claim 11, wherein said method further comprises step B3) determining recognition of cells presenting a T-cell activating antigen by the TCR expressed in step B1).

14. The method of claim 9, wherein said method further comprises step B4) producing a soluble TCR comprising at least the CDRs determined in step B) and determining binding of said soluble TCR to a cancer cell and/or to a cancer antigen complexed in a major histocompatibility complex (MHC) molecule.

15. A method of providing a T-cell recognizing a cell presenting a T-cell activating antigen said method comprising
(i) identifying a TCR binding to a cell presenting a T-cell activating antigen according to the method according to claim 9,
(ii) expressing a TCR comprising at least the complementarity determining regions (CDRs) of the TCR of step (I) in a T-cell, and, thereby,
(iii) providing a T-cell recognizing a cell presenting a T-cell activating antigen.

16. A reactive T-cell identified by the method according to claim 1 and/or obtained or obtainable by the method according to claim 9, for use in medicine or for use in treating and/or preventing cancer in a subject.

17. A method of identifying at least one biomarker of reactive T-cells, comprising
(I) providing expression data of a plurality of biomarkers of T-cells in a sample of a subject,
(II) providing a clustering said plurality of T-cells based on the expression of the biomarkers of step (I);
(III) providing amino acid sequences of at least the complementarity determining regions (CDRs) of TCRes of T-cells of step (II);
(IV) determining reactivity of T-cells expressing a TCR comprising the CDRs of step (III) to cells presenting a T-cell activating antigen;
(V) repeating steps (III) and (IV) at least once for further T-cells clustering with T-cells whose TCRs are determined to be reactive to cells presenting a T-cell activating antigen in step (IV), wherein the TCRs of said further T-cells are non-identical to the TCRs of step (IV);
(VI) determining at least one cluster of step (II) comprising the highest fraction of T-cells comprising T-cell receptors recognizing cells presenting a T-cell activating antigen; and
(VII) determining at least one biomarker expressed by the highest fraction of T-cells in the cluster determined in step (VI), thereby identifying at least one biomarkers of cancer-reactive T-cells.

18. The method of claim 1, wherein said T-cell(s) is/are CD8+ T-cell(s) or CD4+ T-cells.

19. The method of claim 9, wherein said TCR comprises a TCR alpha chain and a TCR beta chain.

20. The method of claim 9, wherein expression of the nucleic acid sequences encoding the amino acid sequences of step (B) is determined by single-cell sequencing.


SEQUENCE LISTING (4 sequences)

SEQ ID NO: 1 - protein, 268 aa, Homo sapiens
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILRVAYSGAGSYQLTFGKGTKLSVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 2 - protein, 315 aa, Homo sapiens
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSRGVAGSSNQPQHFGDGTRLSILEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRKRRGSG

SEQ ID NO: 3 - DNA, 807 bp, Homo sapiens
atgaagttggtgacaagcattactgtactcctatctttgggtattatgggtgatgctaagaccacacagccaaattcaatggagagtaacgaagaagagcctgttcacttgccttgtaaccactccacaatcagtggaactgattacatacattggtatcgacagcttccctcccagggtccagagtacgtgattcatggtcttacaagcaatgtgaacaacagaatggcctctctggcaatcgctgaagacagaaagtccagtaccttgatcctgcaccgtgctaccttgagagatgctgctgtgtactactgcatcctgagagtcgcatactctggggctgggagttaccaactcactttcgggaaggggaccaaactctcggtcataccaaacatccagaatcctgagcctgccgtgtaccagctgaaggaccctagaagccaggacagcaccctgtgcctgttcaccgacttcgacagccagatcaacgtgcccaagaccatggaaagcggcaccttcatcaccgacaagtgtgtgctggacatgaaggccatggacagcaagagcaacggcgccattgcctggtccaaccagaccagcttcacatgccaggacatcttcaaagagacaaacgccacctatcctagcagcgacgtgccctgtgatgccacactgaccgagaagtccttcgagacagacatgaacctgaacttccagaacctgagcgtgatgggcctgagaatcctgctgctgaaggtggccggcttcaacctgctgatgaccctgagactgtggtccagctga

SEQ ID NO: 4 - DNA, 945 bp, Homo sapiens
atgggtcctgggcttctccactggatggccctttgtctccttggaacaggtcatggggatgccatggtcatccagaacccaagataccaggttacccagtttggaaagccagtgaccctgagttgttctcagactttgaaccataacgtcatgtactggtaccagcagaagtcaagtcaggccccaaagctgctgttccactactatgacaaagattttaacaatgaagcagacacccctgataacttccaatccaggaggccgaacacttctttctgctttcttgacatccgctcaccaggcctgggggacgcagccatgtacctgtgtgccaccagcagaggagtggcagggagtagcaatcagccccagcattttggtgatgggactcgactctccatcctagaagatctgcggaacgtgacccctcctaaggtgtccctgttcgagcctagcaaggccgagatcgccaacaagcagaaagccacactcgtgtgcctggccagaggcttctttcccgatcacgtggaactgtcttggtgggtcaacggcaaagaggtgcacagcggcgtctgcacagatccccaggcctacaaagagagcaactacagctactgcctgagcagcagactgagagtgtccgccaccttctggcacaaccccagaaaccacttcagatgccaggtgcagtttcacggcctgagcgaagaggacaagtggcctgagggctctcccaagcctgtgacacagaatatctctgccgaagcctggggcagagccgattgtggaattaccagcgccagctaccagcagggcgtgctgtctgccacaatcctgtacgagatcctgctgggcaaagccactctgtacgccgtgctggtgtctaccctggtcgtgatggccatggtcaagcggaagaacagccggaagagaagaggaagcggc